(19) United States
(12) Patent Application Publication
(10) Pub. No.: US 2023/0265212 A1
(43) Pub. Date: Aug. 24, 2023

(54) COMPOSITIONS AND METHODS FOR OOCYTE-SPECIFIC CONTRACEPTION

(71) Applicants: Kevin S. EISENFRATS, Charlottesville, VA (US); David KROEGER, Victoria (CA); Overa, Inc., Charlottesville, VA (US)

(72) Inventors: David Kroeger, Victoria (CA); Kevin S. Eisenfrats, Charlottesville, VA (US)

(21) Appl. No.: 17/787,773

(22) PCT Filed: Dec. 21, 2020
(86) PCT No.: PCT/US20/66333

§ 371 Date: Jun. 21, 2022

Related U.S. Application Data
(60) Provisional application No. 62/950,706, filed on Dec. 19, 2019.

(51) Int. Cl. C07K 16/40; A61K 31/535

(52) U.S. Cl. CPC C07K 16/40; A61K 31/535; C07K 2317/24; C07K 2317/565

(57) ABSTRACT

Isolated antibodies, antigen-binding portions or fragments thereof or antibody-drug conjugates which bind specifically to a propeptide region of human SAS1B, such as amino acids 34-53 and/or amino acids 64-86 of human SAS1B, are disclosed. Therapeutic and diagnostic applications and methods of use, such as for cancer therapeutics, contraception and fertility, are also disclosed.

### CROSS-REFERENCE TO RELATED APPLICATIONS AND PUBLICATIONS

The present application relies on the disclosure of and claims priority to and the benefit of the filing date of U.S. Provisional Application No. 62/950,706, filed Dec. 19, 2019, the disclosure of which is hereby incorporated by reference herein in its entirety.

The present application is related in subject matter to U.S. Patent Application Publication No. 20160195538A1, U.S. Pat. No. 9,244,075B2, International Patent Application Publication No. WO2012019184A2, U.S. Patent Application Publication No. US20190194345A1, and International Patent Application Publication Nos. WO2017062496A2 and WO2017147247A1. The present application is also related in subject matter to the following publications: Pires E S, D'Souza R S, Needham M A, et al. Membrane associated cancer-oocyte neoantigen SAS1B/ovastacin is a candidate immunotherapeutic target for uterine tumors [published correction appears in Oncotarget. 2017 Feb. 28; 8(9):16099]. Oncotarget. 2015; 6(30):30194-30211. doi:10.18632/oncotarget.4734; Knapp K A, Pires E S, Adair S J, et al. Evaluation of SAS1B as a target for antibody-drug conjugate therapy in the treatment of pancreatic cancer. Oncotarget. 2018; 9(10):8972-8984. Published 2018 Jan. 4. doi:10.18632/oncotarget.23944; Arabinda Mandal, Monika Sachdev, Laura Digilio, Subbarayalu Panneerdoss, Viswanadhapalli Suryavathi, Eusebio Pires, Charles Flickinger, John Herr, SAS1B Is an Egg Specific High Affinity Oolemmal Binding Partner for Sperm Specific Acrosomal SLLP1 During Fertilization, Biology of Reproduction, Volume 85, Issue Suppl_1, 1 Jul. 2011, Page 138, https://doi.org/10.1093/biolreprod/85.s1.138; Sachdev M, Mandal A, Mulders S, et al. Oocyte specific oolemmal SAS1B involved in sperm binding through intra-acrosomal SLLP1 during fertilization. Dev Biol. 2012; 363(1):40-51. doi:10.1016/j.ydbio.2011.12.021; Pires, E. S., Hlavin, C., Macnamara, E., Ishola-Gbenla, K., Doerwaldt, C., Chamberlain, C., Klotz, K., Herr, A. K., Khole, A., Chertihin, O., Curnow, E., Feldman, S. H., Mandal, A., Shetty, J., Flickinger, C. and Herr, J. C. (2013), SAS1B protein [ovastacin] shows temporal and spatial restriction to oocytes in several eutherian orders and initiates translation at the primary to secondary follicle transition. Dev. Dyn., 242: 1405-1426. doi:10.1002/dvdy.24040. Each of these references, patents, published patent applications, and publications are hereby incorporated by reference herein in their entireties.

### REFERENCE TO THE SEQUENCE LISTINGS

The present application contains a Sequence Listing which has been submitted electronically in ASCII format as well as in the attached paper copy. Said ASCII file, created on Dec. 13, 2020, is named OVERA104PCT_ST25.txt and is 20,056 bytes in size. Applicant asserts that the Sequence Listing information recorded in ASCII format is identical to the Sequence Listing information in the attached paper copy. The present application incorporates the Sequence Listing by reference herein in its entirety.

### BACKGROUND OF THE INVENTION

As previously described in US20190194345A1, human SAS1B is expressed via at least six splicing variants SV1-6 leading to corresponding protein isoforms. Four of these contain a short N-terminal intracellular domain of 9 amino acids, a transmembrane region, a proximal membrane region, and the propeptide region followed by C-terminal domains differentially expressed by splice variation of corresponding exons.

Because four of the splice isoforms do not contain a canonical signal peptide and scission sequence, it was anticipated that these proteins become surface bound, tethered to the membrane via the transmembrane region. It was further hypothesized that the proximal membrane region and the propeptide region would remain bound to the cell surface following cleavage of the propeptide from the catalytic domain. While there is evidence that SAS1B tagged on the C-terminal end with the fluorescent protein mCherry is cell surface bound, antibodies generated against the propeptide region would be expected to bind all isoforms of SAS1B and, in addition, bind the residual membrane-tethered propeptide region, whereas antibodies directed at the catalytic domain or the C-terminal domain would not.

Unlike prior attempts to generate anti-SAS1B antibodies, in this application, the present inventors generated antibodies directed against the propeptide region upstream of the cleavage site. The results are antibody compositions that bound specifically to SAS1B on the surface of human oocytes and cells overexpressing SAS1B. These antibodies also demonstrated higher binding affinities than known anti-SAS1B antibodies.

### BRIEF DESCRIPTION OF THE POLYPEPTIDE AND POLYNUCLEOTIDE SEQUENCES

The following sequences are represented in the attached sequence listing. Sequence information for Complementary Determining Regions (CDRs) is also represented in the table in FIG. 20. All sequences are Mus musculus polypeptide or polynucleotide sequences unless otherwise noted.

SEQ ID NOS:1-3 and SEQ ID NOS:6-8 are amino acid sequences of Complementary Determining Regions (CDRs) for monoclonal antibody OV115, where CDR1, CDR2, and CDR3 for variable heavy chain are represented by SEQ ID NO:1, SEQ ID NO:2, and SEQ ID NO:3, respectively, and CDR1, CDR2, and CDR3 for variable light chain are represented by SEQ ID NO:6, SEQ ID NO:7, and SEQ ID NO:8, respectively.

SEQ ID NO:4 represents the full nucleotide sequence for monoclonal antibody OV115, variable heavy chain.

SEQ ID NO:5 represents the full amino acid sequence for monoclonal antibody OV115, variable heavy chain.

SEQ ID NO:9 represents the full nucleotide sequence for monoclonal antibody OV115, variable light chain.

SEQ ID NO:10 represents the full amino acid sequence for monoclonal antibody OV115, variable light chain.

SEQ ID NOS:11-13 and SEQ ID NOS:16, 7, and 18 are amino acid sequences of Complementary Determining Regions (CDRs) for monoclonal antibody OV119, where CDR1, CDR2, and CDR3 for variable heavy chain are represented by SEQ ID NO:11, SEQ ID NO:12, and SEQ ID NO:13, respectively, and CDR1, CDR2, and CDR3 for variable light chain are represented by SEQ ID NO:16, SEQ ID NO:7, and SEQ ID NO:18, respectively.

SEQ ID NO:14 represents the full nucleotide sequence for monoclonal antibody OV119, variable heavy chain.

SEQ ID NO:15 represents the full amino acid sequence for monoclonal antibody OV119, variable heavy chain.

SEQ ID NO:19 represents the full nucleotide sequence for monoclonal antibody OV119, variable light chain.

SEQ ID NO:20 represents the full amino acid sequence for monoclonal antibody OV119, variable light chain.

SEQ ID NOS:21-23 and SEQ ID NOS:26-28 are amino acid sequences of Complementary Determining Regions (CDRs) for monoclonal antibody OV121, where CDR1, CDR2, and CDR3 for variable heavy chain are represented by SEQ ID NO:21, SEQ ID NO:22, and SEQ ID NO:23, respectively, and CDR1, CDR2, and CDR3 for variable light chain are represented by SEQ ID NO:26, SEQ ID NO:27, and SEQ ID NO:28, respectively.

SEQ ID NO:24 represents the full nucleotide sequence for monoclonal antibody OV121, variable heavy chain.

SEQ ID NO:25 represents the full amino acid sequence for monoclonal antibody OV121, variable heavy chain.

SEQ ID NO:29 represents the full nucleotide sequence for monoclonal antibody OV121, variable light chain.

SEQ ID NO:30 represents the full amino acid sequence for monoclonal antibody OV121, variable light chain.

SEQ ID NOS:31-33 and SEQ ID NOS:6, 37, and 8 are amino acid sequences of Complementary Determining Regions (CDRs) for monoclonal antibody OV123, where CDR1, CDR2, and CDR3 for variable heavy chain are represented by SEQ ID NO:31, SEQ ID NO:32, and SEQ ID NO:33, respectively, and CDR1, CDR2, and CDR3 for variable light chain are represented by SEQ ID NO:6, SEQ ID NO:37, and SEQ ID NO:8, respectively.

SEQ ID NO:34 represents the full nucleotide sequence for monoclonal antibody OV123, variable heavy chain.

SEQ ID NO:35 represents the full amino acid sequence for monoclonal antibody OV123, variable heavy chain.

SEQ ID NO:39 represents the full nucleotide sequence for monoclonal antibody OV123, variable light chain.

SEQ ID NO:40 represents the full amino acid sequence for monoclonal antibody OV123, variable light chain.

SEQ ID NOS:41-43 and SEQ ID NOS:46-48 are amino acid sequences of Complementary Determining Regions (CDRs) for monoclonal antibody OV124, where CDR1, CDR2, and CDR3 for variable heavy chain are represented by SEQ ID NO:41, SEQ ID NO:42, and SEQ ID NO:43, respectively, and CDR1, CDR2, and CDR3 for variable light chain are represented by SEQ ID NO:46, SEQ ID NO:47, and SEQ ID NO:48, respectively.

SEQ ID NO:44 represents the full nucleotide sequence for monoclonal antibody OV124, variable heavy chain.

SEQ ID NO:45 represents the full amino acid sequence for monoclonal antibody OV124, variable heavy chain.

SEQ ID NO:49 represents the full nucleotide sequence for monoclonal antibody OV124, variable light chain.

SEQ ID NO:50 represents the full amino acid sequence for monoclonal antibody OV124, variable light chain.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIG. 1A is a fluorescent microscopy image of a human oocyte.

FIG. 1B is a table showing ELISA results for screening antibodies against SAS1B antigen, as discussed in Example 1.

FIGS. 2, 3A and 3B, and 4-6 are fluorescent microscopy images showing Juno visualized by red staining and SAS1B visualized by green staining using anti-SAS1B antibodies, as discussed in Examples 1-3.

FIG. 7 is a graph showing a sensorgram of OV124, as discussed in Example 4.

FIGS. 8 and 9 are tables showing binding constants of antibodies, as discussed in Example 4.

FIG. 10 is a table showing antibodies and their IgG subisotypes, as discussed in Example 6.

FIG. 11 is an illustration showing a biocompatible implant, bulking mechanism, foam, hydrogel, polymer, or other occlusive substance containing one or more antibodies of the invention implanted into a fallopian tube, according to an embodiment.

FIGS. 12A-C are reverse-phase liquid chromatography (LC) plots of OV119 conjugated to DM1 using AJICAP site-specific conjugation, where FIG. 12A shows naked antibody (light chain (LC) and heavy chain (HC)), FIG. 12B shows azide intermediate, and FIG. 12C shows DM1 conjugated antibody, as discussed in Example 7.

FIGS. 13A-13C are quantitative time of flight traces showing OV119 conjugated to DM1, as discussed in Example 8. OV119 was analyzed under native (FIG. 13A) and reducing conditions for LC (FIG. 13B) and HC (FIG. 13C). The top traces represents naked antibody, while the bottom traces represent DM1 conjugated antibody.

FIG. 14 is a graph showing colorimetric ELISA data comparing binding of naked OV119 (unconjugated) to OV119 conjugated using stochastic chemistry (OV119-WT-OM) and AJICAP site-specific conjugation (OV119-CAP-DM1), as discussed in Example 9.

FIG. 15 (see also, FIG. 1A) is a fluorescence microscopy image showing surface staining of SAS1B on live human oocyte (OV119), as discussed in Example 10.

FIGS. 16A-C are compressed confocal microscopy image stacks of OV119, OV119-DM1 and EEA1 on permeabilized human oocytes, as discussed in Example 11, where control mAb (FIG. 16A), OV119 (FIG. 16B), and OV119-DM1 (FIG. 16C) were stained green. Red stain represents EEA1 while blue stain represents DAPI in each image.

FIGS. 17A-D are microscopy images showing evidence for internalization of OV119-DM1 and colocalization with EEA1 (entry to early endosomes), as discussed in Example 12, where FIG. 17A is a compressed confocal image of a OV119-DM1-stained oocyte, FIG. 17B shows Z-plane cross-sections of the same oocyte zoomed to show co-localization (appears yellow). FIGS. 17C and 17D are single z-plane confocal images showing co-localization between OV119-DM1 and EEA1.

FIGS. 18A-C are fluorescence microscopy images showing evidence for disruption of the meiotic spindle (MII) following exposure of human oocytes to OV119-DM1, as discussed in Example 13, where FIG. 18A represents non-treated control, FIG. 18B represents GMA640-DM1 (ADC of irrelevant specificity), and FIG. 18C represents OV119-DM1.

FIG. 19 is a microscopy image with immunohistochemistry showing that OV119 binds oocytes in a stage-dependent manner (ovary from Macaca nemistrina), as discussed in Example 14.

FIG. 20 is a table showing amino acid sequences of complementary determining regions (CDRs) of monoclonal antibodies OV115, OV119, OV121, OV123, and OV124.

FIG. 21 is the chemical structure of DM1, an antibody-conjugatable cytotoxic compound that targets microtubules (from Lopus M. Antibody-DM1 conjugates as cancer therapeutics. Cancer Lett. 2011; 307(2):113-118. doi:10.1016/j.canlet.2011.03.017, incorporated by reference herein).

FIG. 22 is a chart showing antibody and ADC titers over the course of 28 days, as assessed by antigen-specific serum ELISAs, from Balb/c mice injected at day 0 with 3 mg/kg of the indicated molecule.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

As used herein, the singular forms “a,” “an,” and “the” include plural referents unless the context clearly dictates otherwise. The term “about” in association with a numerical value means that the numerical value can vary plus or minus by 5% or less of the numerical value.

In the following detailed description, numerous specific embodiments are set forth in order to provide a thorough understanding of the compositions and methods disclosed herein. However, as will be apparent to those skilled in the art, the present embodiments may be practiced without these specific details or by using alternate elements or processes. In other instances, well-known processes, procedures, and/or components have not been described in detail so as not to unnecessarily obscure aspects of embodiments disclosed herein.

Features regarding exemplary isolated antibodies or antigen-binding portions thereof are described followed by features relating to compositions and methods for detecting and treating cancer, determining fertility, and producing contraception.

As used herein, the designations “OV115”, “OV119”, “OV121”, “OV123”, and “OV124” refer both to the monoclonal antibodies identified as embodiments of the invention as well as the hybridoma clones isolated to produce them.

Embodiments, or Implementations, of the invention include the following:

Embodiment 1. An isolated antibody or antigen-binding portion thereof that specifically binds human SAS1B.

Embodiment 2. An isolated antibody or antigen-binding portion thereof that specifically binds a propeptide region of human SAS1B.

Embodiment 3. An isolated antibody or antigen-binding portion thereof, wherein said antibody or antigen-binding portion thereof specifically binds to a polypeptide comprising or consisting of amino acids 34-53 and/or 64-86 of human SAS1B.

Embodiment 4. An isolated antibody or antigen-binding portion thereof of any preceding Embodiment, wherein the antibody or antigen-binding portion thereof is a monoclonal antibody, a chimeric antibody, a humanized antibody, a synthetic antibody, a single chain antibody, a diabody, or a CDR-grafted antibody.

Embodiment 5. An isolated antibody or antigen-binding portion thereof comprising OV115, OV119, OV121, OV123, and/or OV124.

Embodiment 6. An isolated antibody or antigen-binding portion thereof comprising OV115, OV119, OV121, OV123, and/or OV124, or an isolated antibody or antigen-binding portion with an amino acid sequence with at least 95% identity to the amino acid sequence of OV115, OV119, OV121, OV123, and/or OV124.

Embodiment 7. An isolated antibody or antigen-binding portion thereof comprising one or more of a VH CDR1, a VH CDR2, a VH CDR3, a VL CDR1, a VL CDR2, and/or a VL CDR3 of OV115, OV119, OV121, OV123, and/or OV124, or comprising one or more amino acid sequence with at least 95% identity to a VH CDR1, a VH CDR2, a VH CDR3, a VL CDR1, a VL CDR2, and/or a VL CDR3 of OV115, OV119, OV121, OV123, and/or OV124.

Embodiment 8. An isolated antibody or antigen-binding portion thereof that specifically binds a propeptide region of human SAS1B, wherein said antibody binds the same human SAS1B epitope recognized by a monoclonal antibody produced by a hybridoma cell line described herein, such as OV115, OV119, OV121, OV123, and/or OV124.

Embodiment 9. An isolated antibody or antigen-binding portion thereof, wherein said antibody or antigen-binding portion specifically binds to a propeptide region of human SAS1B, wherein the antibody or antigen-binding portion thereof competes for binding with an antibody or antigen-binding portion thereof comprising OV115, OV119, OV121, OV123, and/or OV124.

Embodiment 10. An isolated antibody or antigen-binding portion thereof, wherein said antibody or antigen-binding portion thereof inhibits binding of OV115, OV119, OV121, OV123, and/or OV124 to human SAS1B.

Embodiment 11. The isolated antibody or antigen-binding portion thereof of any preceding Embodiment wherein said antibody or antigen-binding portion thereof specifically binds to a polypeptide comprising or consisting of amino acids 34-53 and/or 64-86 of human SAS1B.

Embodiment 12. The isolated antibody or antigen-binding portion thereof of any preceding Embodiment, wherein the antibody or antigen-binding portion thereof is a monoclonal antibody, a chimeric antibody, a humanized antibody, a synthetic antibody, a single chain antibody, a diabody, or a CDR-grafted antibody.

Embodiment 13. A composition comprising: the antibody or antigen-binding portion thereof of any preceding Embodiment and a pharmaceutically acceptable carrier; the antibody or antigen-binding portion thereof of any preceding Embodiment, wherein the antibody or antigen-binding portion thereof is conjugated to a therapeutic agent, and a pharmaceutically acceptable carrier; or the antibody or antigen-binding portion thereof of any preceding Embodiment, wherein the antibody or antigen-binding portion thereof is conjugated to a diagnostic or imaging agent.

Embodiment 14. An isolated antibody or antigen-binding portion thereof having an amino acid sequence disclosed herein, or having an amino acid sequence at least 70%, 75%, 80%, 85%, 90%, 95%, or 99% identical to an amino acid sequence disclosed herein.

Embodiment 15. An isolated antibody or antigen-binding portion thereof encoded by a nucleic acid sequence disclosed herein, or having a nucleic acid sequence at least 70%, 75%, 80%, 85%, 90%, 95%, or 99% identical to a nucleic acid sequence disclosed herein.

Embodiment 16. An antibody-drug conjugate (ADC) comprising: the antibody or antigen-binding portion thereof of any preceding Embodiment, wherein the antibody or antigen-binding portion is conjugated to a therapeutic agent.

Embodiment 17. The isolated antibody or antigen-binding portion thereof of any preceding Embodiment, wherein the antibody or antigen-binding portion thereof specifically binds human SAS1B with an affinity (Kd) of at least about 10⁻⁶ M, or at least about 10⁻⁷ M, or at least about 10⁻⁸ M, or at least about 10⁻⁹ M, or at least about 10⁻¹⁰ M, or at least about 10⁻¹¹ M, or at least about 10⁻¹² M.

Embodiment 18. The isolated antibody or antigen-binding portion thereof of any preceding Embodiment, wherein said antibody or antigen-binding portion thereof binds to cancer cells and/or human oocytes.

Embodiment 19. An isolated cell of a hybridoma disclosed herein.

Embodiment 20. An isolated polypeptide having an amino acid sequence disclosed herein.

Embodiment 21. An isolated polynucleotide encoding the polypeptide of Embodiment 20.

Embodiment 22. An isolated polynucleotide encoding an anti-human SAS1B antibody or antigen-binding portion thereof, such as the anti-human SAS1B antibody OV115, OV119, OV121, OV123, and/or OV124.

Embodiment 23. A vector comprising one or more polynucleotides of Embodiment 22.

Embodiment 24. A host cell comprising the vector of Embodiment 23.

Embodiment 25. A method for producing a human SAS1B antibody or antigen-binding portion thereof, comprising: culturing an isolated host cell of Embodiment 24 and recovering said antibody.

Embodiment 26. An isolated antibody or antigen-binding portion thereof of any preceding Embodiment, wherein the antibody is a chimeric antibody comprising VL and VH domains obtained from a mouse antibody, wherein said VL and VH domains include sequences capable of binding to human SAS1B, and the VL and VH domains are fused to human CL and CH domains, respectively.

Embodiment 27. A method of treating or diagnosing a mammal comprising: administering the composition of Embodiment 13 to a mammal in need thereof.

Embodiment 28. A method of detecting a SASB1 polypeptide in a sample, the method comprising: contacting one or more antibodies of any preceding Embodiment with a test sample under conditions that allow polypeptide/antibody complexes to form; and detecting polypeptide/antibody complexes; wherein the detection of polypeptide/antibody complexes is an indication that the human SAS1B polypeptide is present in the test sample.

Embodiment 29. A method of detecting SAS1B-positive cells in a test sample comprising: contacting one or more antibodies of any preceding Embodiment with the test sample under conditions that allow SAS1B-positive cell/antibody complexes to form; and detecting SAS1B positive cell/antibody complexes; wherein the detection of SAS1B positive cell/antibody complexes is an indication that SAS1B cells are present in the test sample.

Embodiment 30. The method of Embodiment 28 or Embodiment 29, wherein the test sample comprises lymph node or tissue aspirate, serum, whole blood, cellular suspension, lymphocytes, whole blood, plasma, circulating tumor cells, tumor cells or tissue, ascites fluid, urine, or fluid effusion.

Embodiment 31. The method of Embodiment 28 or Embodiment 29, wherein the test sample comprises an oocyte (GV, M1, or M2) or ovary or portion thereof.

Embodiment 32. The method of Embodiment 31, wherein the detection of SAS1B positive cell/antibody complexes or polypeptide/antibody complexes or lack thereof provides an indication of fertility or infertility.

Embodiment 33. The method of Embodiment 28 or Embodiment 29, wherein said sample comprises a tumor biopsy, tissue sample, blood, plasma, peritoneal fluid, follicular fluid, ascites, urine, feces, saliva, mucus, phlegm, sputum, tears, cerebrospinal fluid, effusions, lavage, or Pap smears.

Embodiment 34. The method of Embodiment 30 or Embodiment 33, wherein the detection of SAS1B positive cell/antibody or polypeptide/antibody complexes indicates the presence of cancer.

Embodiment 35. The method of Embodiment 34, wherein the cancer is lung cancer, MMMT, bladder cancer, ovarian cancer, uterine cancer, endometrial cancer, breast cancer, head and neck cancer, liver cancer, pancreatic cancer, esophageal cancer, stomach cancer, cervical cancer, prostate cancer, adrenal cancer, lymphoma, leukemia, salivary gland cancer, bone cancer, brain cancer, cerebellar cancer, colon cancer, rectal cancer, colorectal cancer, oronasopharyngeal cancer, NPC, kidney cancer, skin cancer, melanoma, basal cell carcinoma, hard palate carcinoma, squamous cell carcinoma of the tongue, meningioma, pleomorphic adenoma, astrocytoma, chondrosarcoma, cortical adenoma, hepatocellular carcinoma, pancreatic cancer, squamous cell carcinoma, or adenocarcinoma.

Embodiment 36. A method for inhibiting proliferation or killing a SAS1B positive cancer cell, said method comprising contacting said cancer cell with an effective amount of an antibody or antigen-binding portion thereof or antibody-drug conjugate of any preceding Embodiment, wherein said antibody or antigen-binding fragment thereof or antibody-drug conjugate binds with SAS1B, thereby inhibiting proliferation of the cancer cell or killing the cancer cell.

Embodiment 37. The method of Embodiment 36, wherein said killing is antibody-mediated complement-dependent cell killing.

Embodiment 38. The method of Embodiment 36, wherein said cancer is carcinoma, sarcoma, uterine cancer, ovarian cancer, lung cancer, adenocarcinoma, adenocarcinoma of the lung, squamous carcinoma, squamous carcinoma of the lung, malignant mixed mullerian tumor, leukemia, lymphoma, or endometrioid carcinoma.

Embodiment 39. The method of Embodiment 36, wherein said antibody is conjugated to a second molecule or structure.

Embodiment 40. The method of Embodiment 36, wherein said molecule or structure is an antibody, a protein, a pro-drug, a drug, a toxin, a protein toxin, a liposome, a radioactive isotope, or an enzyme.

Embodiment 41. A method for diagnosing cancer in a subject, said method comprising detecting the presence of a cancer-oocyte antigen such as SAS1B in a sample from said subject, wherein the presence of the cancer-oocyte antigen in the sample indicates that the subject has cancer.

Embodiment 42. The method of Embodiment 41, wherein the cancer is lung cancer, MMMT, bladder cancer, ovarian cancer, uterine cancer, endometrial cancer, breast cancer, head and neck cancer, liver cancer, pancreatic cancer, esophageal cancer, stomach cancer, cervical cancer, prostate cancer, adrenal cancer, lymphoma, leukemia, salivary gland cancer, bone cancer, brain cancer, cerebellar cancer, colon cancer, rectal cancer, colorectal cancer, oronasopharyngeal cancer, NPC, kidney cancer, skin cancer, melanoma, basal cell carcinoma, hard palate carcinoma, squamous cell carcinoma of the tongue, meningioma, pleomorphic adenoma, astrocytoma, chondrosarcoma, cortical adenoma, hepatocellular carcinoma, pancreatic cancer, squamous cell carcinoma, or adenocarcinoma.

Embodiment 43. A method for modulating fertility of a subject comprising administering to the subject an amount of a polypeptide comprising or consisting of amino acids 34-53 and/or 64-86 of human SAS1B that is effective to produce an immune response in the subject.

Embodiment 44. The method of Embodiment 43, wherein the immune response is directed toward an oocyte of the subject.

Embodiment 45. The method of Embodiment 44, wherein the immune response is sufficient to: modulate SAS1B present on the oocyte of the subject; inhibit fertilization of the oocyte of the subject; prevent implantation of the oocyte of the subject; disrupt meiosis of an oocyte of the subject; and/or kill the oocyte of the subject.

Embodiment 46. A method for modulating fertility of a subject comprising administering to the subject an amount of an isolated antibody or antigen-binding portion thereof of any preceding Embodiment or antibody-drug conjugate (ADC) of any preceding Embodiment that is sufficient to: modulate SAS1B present on an oocyte of the subject; inhibit fertilization of an oocyte of the subject; Prevent implantation of an oocyte of the subject; disrupt meiosis of an oocyte of the subject; and/or kill the oocyte of the subject.

Embodiment 47. The method of Embodiment 46, wherein the isolated antibody or antigen-binding portion thereof of any preceding Embodiment or antibody-drug conjugate (ADC) of any preceding Embodiment is administered systemically to the subject.

Embodiment 48. The method of Embodiment 46, wherein the isolated antibody or antigen-binding portion thereof of any preceding Embodiment or antibody-drug conjugate (ADC) of any preceding Embodiment is administered locally to the subject, such as within a fallopian tube of the subject.

Embodiment 49. The method of any one of Embodiments 46-48, wherein the isolated antibody or antigen-binding portion thereof of any preceding Embodiment or antibody-drug conjugate (ADC) of any preceding Embodiment is administered as a biocompatible implant, bulking mechanism, foam, hydrogel, polymer, or other occlusive substance which releases the antibody or antigen-binding portion thereof or antibody-drug conjugate (ADC).

Embodiment 50. A vaccine comprising a polypeptide comprising or consisting of amino acids 34-53 and/or 64-86 of human SAS1B and a pharmaceutically acceptable carrier or adjuvant.

Embodiment 51. A method of detecting SAS1B-positive cells in a test sample, the method comprising: determining a topology of an SAS1B molecule at SAS1B-positive cell surfaces by defining alternative splice variants that function as integral membrane proteins; and mapping surface accessible epitopes recognized by an isolated antibody or antigen-binding portion thereof.

Embodiment 52. The method of Embodiment 51, wherein the isolated antibody or antigen-binding portion is a therapeutic monoclonal antibody-drug conjugate (ADC) and/or T-cell immunotherapy that targets SAS1B-positive cell surface epitopes of a SAS1B metalloprotease.

Embodiment 53. The method of Embodiment 51, wherein the alternative splice variants are human astacin-like (ASTL) gene splice variants encoding at least one SAS1B protein isoform to traffic at least one site of the SAS1B-positive cell.

Embodiment 54. The method of Embodiment 53, wherein the at least one site is a plasma membrane.

Embodiment 55. The composition of Embodiment 13, wherein the therapeutic agent is cytotoxic to oocytes and/or cancer cells.

Embodiment 56. The antibody-drug conjugate of Embodiment 16, wherein the therapeutic agent is cytotoxic to oocytes and/or cancer cells

Embodiment 57. The method of Embodiment 39, wherein the second molecule or structure is cytotoxic to oocytes and/or cancer cells.

Embodiment 58. The method of Embodiment 40, wherein the drug is cytotoxic to oocytes and/or cancer cells

Embodiment 59. The method of Embodiment 46, wherein the antibody-drug conjugate is cytotoxic to oocytes and/or cancer cells.

Embodiment 60. The composition, antibody-drug conjugate, or method of any one of Embodiments 55-59, wherein the therapeutic agent, second molecule or structure, or drug is an antibody, a protein, a pro-drug, a drug, a toxin, a protein toxin, a liposome, a radioactive isotope, or an enzyme

Embodiment 61. The composition, antibody-drug conjugate, or method of any one of Embodiments 55-59, wherein the therapeutic agent, second molecule or structure, or drug is an antibody-conjugatable tubulin inhibitor.

Embodiment 62. The composition, antibody-drug conjugate, or method of Embodiment 61, wherein the tubulin inhibitor is a maytansinoid, such as DM1 or DM4.

Embodiment 63. The isolated antibody or antigen-binding portion of any one of Embodiments 1-12, 14, 15, 17, 18, and 26, comprising one or more variable heavy chain CDRs having an amino acid sequence 100% identical to, or 60%, 65%, 70%, 75%, 80%, 85%, 90%, 95%, or 99% identical to, SEQ ID NO:1, SEQ ID NO:2, SEQ ID NO:3, SEQ ID NO:11, SEQ ID NO:12, SEQ ID NO:13, SEQ ID NO:21, SEQ ID NO:22, SEQ ID NO:23, SEQ ID NO:31, SEQ ID NO:32, SEQ ID NO:33, SEQ ID NO:41, SEQ ID NO:42, and/or SEQ ID NO:43.

Embodiment 64. The isolated antibody or antigen-binding portion of Embodiment 63, wherein the isolated antibody or antigen binding portion specifically binds human SAS1B, and/or a polypeptide comprising or consisting of amino acids 34-53 and/or 64-86 of human SAS1B, with an affinity (Kd) of at least about 10⁻⁶ M, or at least about 10⁻⁷ M, or at least about 10⁻⁸ M, or at least about 10⁻⁹ M, or at least about 10⁻¹⁰ M, or at least about 10⁻¹¹ M, or at least about 10⁻¹² M.

Embodiment 65. The isolated antibody or antigen-binding portion of any one of Embodiments 1-12, 14, 15, 17, 18, and 26, comprising one or more variable light chain CDRs having an amino acid sequence 100% identical to, or 60%, 65%, 70%, 75%, 80%, 85%, 90%, 95%, or 99% identical to, SEQ ID NO:6, SEQ ID NO:7, SEQ ID NO:8, SEQ ID NO:16, SEQ ID NO:18, SEQ ID NO:26, SEQ ID NO:27, SEQ ID NO:28, SEQ ID NO:37, SEQ ID NO:46, SEQ ID NO:47, and/or SEQ ID NO:48.

Embodiment 66. The isolated antibody or antigen-binding portion of Embodiment 65, wherein the isolated antibody or antigen binding portion specifically binds human SAS1B, or a polypeptide comprising or consisting of amino acids 34-53 and/or 64-86 of human SAS1B, with an affinity (Kd) of at least about 10-6 M, or at least about 10-7 M, or at least about 10-8 M, or at least about 10⁻⁹ M, or at least about 10-10 M, or at least about 10-11 M, and/or at least about 10-12 M.

Embodiment 67. An isolated antibody or antigen-binding portion thereof comprising: one or more antibody amino acid sequences selected from:

a. variable heavy chain CDRs represented by SEQ ID NO:1, SEQ ID NO:2, SEQ ID NO:3, SEQ ID NO:11, SEQ ID NO:12, SEQ ID NO:13, SEQ ID NO:21, SEQ ID NO:22, SEQ ID NO:23, SEQ ID NO:31, SEQ ID NO:32, SEQ ID NO:33, SEQ ID NO:41, SEQ ID NO:42, and SEQ ID NO:43; and

b. variable light chain CDRs represented by SEQ ID NO:6, SEQ ID NO:7, SEQ ID NO:8, SEQ ID NO:16, SEQ ID NO:18, SEQ ID NO:26, SEQ ID NO:27, SEQ ID NO:28, SEQ ID NO:37, SEQ ID NO:46, SEQ ID NO:47, and SEQ ID NO:48;

wherein one or more of the selected antibody amino acid sequences listed in (a) or (b) are between 60% and 100% identical, inclusive, to those set forth, such as 60%, 65%, 70%, 75%, 80%, 85%, 90%, 95%, 99%, or 100% identical, and can have one or more amino acid substitutions, additions, or deletions, such as from 1 to 3, 1 to 4, 1 to 5, 1 to 6, 1 to 7, 1 to 8, 1 to 9, 1 to 10, or 1 to 11 amino acid substitutions, additions, or deletions; and

wherein the isolated antibody or antigen-binding portion specifically binds human SAS1B.

Embodiment 68. The isolated antibody or antigen-binding portion of Embodiment 67, wherein the antibody or antigen-binding portion thereof specifically binds to a polypeptide comprising or consisting of amino acids 34-53 and/or 64-86 of human SAS1B.

Embodiment 69. The isolated antibody or antigen-binding portion of Embodiment 67 or Embodiment 68, wherein the antibody or antigen-binding portion specifically binds human SAS1B, and/or a polypeptide comprising or consisting of amino acids 34-53 and/or 64-86 of human SAS1B, with an affinity (Kd) of at least about 10⁻⁶ M, or at least about 10⁻⁷ M, or at least about 10⁻⁸ M, or at least about 10⁻⁹ M, or at least about 10⁻¹⁰ M, or at least about 10⁻¹¹ M, or at least about 10⁻¹² M.

Embodiment 70. The isolated antibody or antigen-binding portion of Embodiment 67, wherein the selected antibody amino acid sequences listed in (a) or (b) are 100% identical to those set forth, having no amino acid substitution, addition, or deletion.

Embodiment 71. The isolated antibody or antigen-binding portion of Embodiment 67, comprising OV115, OV119, OV121, OV123, and/or OV124.

Embodiment 72. An isolated antibody or antigen-binding portion thereof comprising:

a chimeric polypeptide comprising:

a human antibody amino acid sequence or sequences comprising one or more constant regions, such as human constant light (CL) chain and/or constant heavy (CH) chain domains, a framework region of a heavy chain variable region, and/or a framework region of a light chain variable region; and

one or more non-human, such as mouse, antibody amino acid sequences selected from:

(a) variable heavy chain CDRs represented by SEQ ID NO:1, SEQ ID NO:2, SEQ ID NO:3, SEQ ID NO:11, SEQ ID NO:12, SEQ ID NO:13, SEQ ID NO:21, SEQ ID NO:22, SEQ ID NO:23, SEQ ID NO:31, SEQ ID NO:32, SEQ ID NO:33, SEQ ID NO:41, SEQ ID NO:42, and SEQ ID NO:43; and

(b) variable light chain CDRs represented by SEQ ID NO:6, SEQ ID NO:7, SEQ ID NO:8, SEQ ID NO:16, SEQ ID NO:18, SEQ ID NO:26, SEQ ID NO:27, SEQ ID NO:28, SEQ ID NO:37, SEQ ID NO:46, SEQ ID NO:47, and SEQ ID NO:48;

wherein one or more of the selected non-human antibody amino acid sequences listed in (a) or (b) are between 60% and 100% identical, inclusive, to those set forth, such as 60%, 65%, 70%, 75%, 80%, 85%, 90%, 95%, 99%, or 100% identical, and can have one or more amino acid substitutions, additions, or deletions, such as from 1 to 3, 1 to 4, 1 to 5, 1 to 6, 1 to 7, 1 to 8, 1 to 9, 1 to 10, or 1 to 11 amino acid substitutions, additions or deletions; and

wherein the antibody or antigen-binding portion thereof specifically binds human SAS1B and/or a polypeptide comprising or consisting of amino acids 34-53 and/or 64-86 of human SAS1B.

Embodiment 73. The antibody or antigen-binding portion thereof of Embodiment 72, wherein the selected non-human antibody amino acid sequences listed in (a) or (b) are 100% identical to those set forth, having no amino acid substitution, addition, or deletion.

Embodiment 74. The antibody or antigen-binding portion thereof of Embodiment 72, wherein the antibody or antigen-binding portion thereof specifically binds human SAS1B and/or a polypeptide comprising or consisting of amino acids 34-53 and/or 64-86 of human SAS1B with an affinity (Kd) of at least about 10⁻⁶ M, or at least about 10⁻⁷ M, or at least about 10⁻⁸ M, or at least about 10⁻⁹ M, or at least about 10⁻¹⁰ M, or at least about 10⁻¹¹ M, or at least about 10⁻¹² M.

Embodiment 75. A therapeutic agent comprising the antibody or antigen-binding portion thereof of Embodiment 67 or Embodiment 72 conjugated to a molecule or structure that is cytotoxic to oocytes and/or cancer cells.

Embodiment 76. The therapeutic agent of Embodiment 75, wherein the molecule or structure is an antibody, a protein, a pro-drug, a drug, a toxin, a protein toxin, a liposome, a radioactive isotope, or an enzyme.

Embodiment 77. The therapeutic agent of Embodiment 75, wherein the molecule or structure is an antibody-conjugatable tubulin inhibitor.

Embodiment 78. The therapeutic agent of Embodiment 77, wherein the tubulin inhibitor is a maytansinoid, such as DM1 or DM4.

Embodiment 79. A composition comprising the therapeutic agent of Embodiment 75 and a pharmaceutically acceptable carrier.

Embodiment 80. A composition comprising the therapeutic agent of Embodiment 75 within a biocompatible implant, bulking mechanism, foam, hydrogel, polymer, or other occlusive substance.

Embodiment 81. A method for inhibiting proliferation or killing a SAS1B positive cancer cell, the method comprising contacting the cancer cell with an effective amount of an antibody or antigen-binding portion thereof of Embodiment 67 or 72, or therapeutic agent of Embodiment 75, wherein said antibody or antigen-binding fragment thereof or therapeutic agent binds with SAS1B, thereby inhibiting proliferation of the cancer cell or killing the cancer cell.

Embodiment 82. The method of Embodiment 81, wherein the killing is antibody-mediated complement-dependent cell killing.

Embodiment 83. The method of Embodiment 81, wherein the cancer is carcinoma, sarcoma, uterine cancer, ovarian cancer, lung cancer, adenocarcinoma, adenocarcinoma of the lung, squamous carcinoma, squamous carcinoma of the lung, malignant mixed mullerian tumor, leukemia, lymphoma, or endometrioid carcinoma.

Embodiment 84. The method of Embodiment 81, wherein the molecule or structure is an antibody, a protein, a pro-drug, a drug, a toxin, a protein toxin, a liposome, a radioactive isotope, or an enzyme.

Embodiment 85. The method of Embodiment 81, wherein the molecule or structure is an antibody-conjugatable tubulin inhibitor.

Embodiment 86. The method of Embodiment 85, wherein the tubulin inhibitor is a maytansinoid, such as DM1 or DM4.

Embodiment 87. A method for modulating fertility of a subject comprising administering to the subject an amount of an isolated antibody or antigen-binding portion thereof of Embodiment 67 or Embodiment 72, or therapeutic agent of Embodiment 75 that is sufficient to: modulate SAS1B present on an oocyte of the subject; inhibit fertilization of an oocyte of the subject; prevent implantation of an oocyte of the subject; disrupt meiosis of an oocyte of the subject; and/or kill the oocyte of the subject.

Embodiment 88. The method of Embodiment 87, wherein the isolated antibody or antigen-binding portion thereof of or therapeutic agent is administered systemically to the subject.

Embodiment 89. The method of Embodiment 87, wherein the isolated antibody or antigen-binding portion thereof or therapeutic agent is administered locally to the subject, such as within a fallopian tube of the subject.

Embodiment 90. The method of Embodiment 87, wherein the isolated antibody or antigen-binding portion thereof of or therapeutic agent is administered within a biocompatible implant, bulking mechanism, foam, hydrogel, polymer, or other occlusive substance which releases the antibody or antigen-binding portion thereof or therapeutic agent.

Embodiment 91. The method of Embodiment 87, wherein the molecule or structure of the therapeutic agent is an antibody, a protein, a pro-drug, a drug, a toxin, a protein toxin, a liposome, a radioactive isotope, or an enzyme.

Embodiment 92. The method of Embodiment 87, wherein the molecule or structure of the therapeutic agent is an antibody-conjugatable tubulin inhibitor.

Embodiment 93. The method of Embodiment 92, wherein the tubulin inhibitor is a maytansinoid, such as DM1 or DM4.

Embodiment 94. An isolated polynucleotide encoding the antibody or antigen-binding portion thereof of Embodiment 67 or Embodiment 72 or one or more polypeptide chain, domain, or region thereof.

Embodiment 95. An isolated polynucleotide encoding a polypeptide with the amino acid sequence set forth in SEQ ID NO:5, SEQ ID NO:10, SEQ ID NO:15, SEQ ID NO:20, SEQ ID NO:25, SEQ ID NO:30, SEQ ID NO:35, SEQ ID NO:40, SEQ ID NO:45, and/or SEQ ID NO:50.

Embodiment 96. An isolated polynucleotide having a nucleotide sequence set forth in SEQ ID NO:4, SEQ ID NO:9, SEQ ID NO:14, SEQ ID NO:19, SEQ ID NO:24, SEQ ID NO:29, SEQ ID NO:34, SEQ ID NO:39, SEQ ID NO:44, and/or SEQ ID NO:49, or a polynucleotide variant having 60%, 65%, 70%, 75%, 80%, 85%, 90%, 95%, or 99% identity thereto.

Embodiment 97. An isolated polypeptide having the amino acid sequence set forth in SEQ ID NO:5, SEQ ID NO:10, SEQ ID NO:15, SEQ ID NO:20, SEQ ID NO:25, SEQ ID NO:30, SEQ ID NO:35, SEQ ID NO:40, SEQ ID NO:45, and/or SEQ ID NO:50, or a polypeptide variant having 60%, 65%, 70%, 75%, 80%, 85%, 90%, 95%, or 99% identity thereto.

Embodiment 98. A vector comprising one or more polynucleotides of any one of Embodiments 94-96.

Embodiment 99. A host cell comprising the vector of Embodiment 98.

Embodiment 100. A method for producing a human SAS1B antibody or antigen-binding portion thereof, comprising: culturing an isolated host cell of Embodiment 99 and recovering said antibody or antigen-binding portion.

Embodiment 101. A method of treating or diagnosing a mammal comprising: administering the isolated antibody or antigen-binding portion thereof of Embodiment 67 or Embodiment 72 to a mammal in need thereof.

This disclosure provides antibodies and antigen-binding portions thereof that specifically bind to the human metallo-endoprotease SAS1B, a product of the ASTL gene. These antibodies and antigen-binding portions thereof have applications in diagnostic assays to measure SAS1B such as in fertility and/or cancer applications. They also have applications as therapeutic probes, both alone, as “naked” unconjugated antibodies, and conjugated with cytotoxic drugs and/or radionuclides such as for cancer therapeutics and/or contraception. These antibodies also have therapeutic use as imaging agents.

These and additional Embodiments, or Implementations, are further described below.

Isolated Antibodies or Antigen-Binding Portions Thereof

Polypeptides

A polypeptide is a polymer of three or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure.

A light or heavy chain variable region of an antibody has four framework regions interrupted by three hypervariable regions, known as complementary determining regions (CDRs). CDRs determine the specificity of antigen binding. The heavy chain and light chain each have three CDRs, designated from the N terminus as CDR1, CDR2, and CDR3 with the four framework regions flanking these CDRs. The amino acid sequences of the framework region are highly conserved and CDRs can be transplanted into other antibodies. Therefore, a recombinant antibody can be produced by combining CDRs from one or more antibodies with the framework of one or more other antibodies. Antibodies of the disclosure include antibodies that comprise at least one, two, three, four, five, or six (or combinations thereof) of the CDRs of any of the monoclonal antibodies isolated from the hybridomas identified in the Examples (e.g. OV115, OV119, OV121, OV123, and/or OV124), or variant CDRs. Variant CDRs are CDRs comprising amino acid sequences similar to the amino acid sequences of CDRs of any of the monoclonal antibodies produced by the hybridomas identified in the Examples (e.g. OV115, OV119, OV121, OV123, and/or OV124). Polypeptides of the present disclosure comprise full-length human, mouse, or rabbit anti-SAS1B heavy chain variable regions, full-length human, mouse or rabbit light chain regions, fragments thereof, and combinations thereof.

An antibody of the present disclosure can comprise a VH (variable heavy chain), a VL (variable light chain), a VH CDR1, a VH CDR 2, a VH CDR 3, a VL CDR 1, a VL CDR 2, a VL CDR3, of any antibody amino acid sequence provided in this disclosure (e.g. OV115, OV119, OV121, OV123, and/or OV124). An antibody of the present disclosure can have any combination of VH CDR1, VH CDR2, VH CDR3, VL CDR1, VL CDR2, VL CDR3, variant VH CDR1, variant VH CDR2, variant VH CDR3, variant VL CDR1, variant VL CDR2, or variant VL CDR3 of any antibody amino acid sequence provided in this disclosure (e.g. OV115, OV119, OV121, OV123, and/or OV124).

An antibody of the present disclosure can comprise a VH nucleic acid sequence or a VL nucleic acid sequence of any nucleic acid sequence provided in this disclosure.

An antibody of the present disclosure can comprise the variable heavy chain CDRs from antibody OV115, OV119, OV121, OV123, and/or OV124. An antibody of the present disclosure can comprise the variable light chain CDRs from antibody OV115, OV119, OV121, OV123, and/or OV124. An antibody of the present disclosure can comprise a variable light chain that comprises the amino acid sequence of at least one or at least two or at least 3 CDRs of the antibody variable light chains from antibody OV115, OV119, OV121, OV123, and/or OV124. An antibody of the present disclosure can comprise a variable heavy chain that comprises the amino acid sequence of at least one or at least two or at least 3 CDRs of the antibody variable heavy chains from antibody OV115, OV119, OV121, OV123, and/or OV124.

Any antibody or antigen-binding portion (e.g. antibody fragment or antigen-binding fragment) of the present disclosure can include one or more heavy chain CDRs with amino acid sequences represented by SEQ ID NO:1, SEQ ID NO:2, SEQ ID NO:3, SEQ ID NO:11, SEQ ID NO:12, SEQ ID NO:13, SEQ ID NO:21, SEQ ID NO:22, SEQ ID NO:23, SEQ ID NO:31, SEQ ID NO:32, SEQ ID NO:33, SEQ ID NO:41, SEQ ID NO:42, and/or SEQ ID NO:43, and/or one or more light chain CDRs with amino acid sequences represented by SEQ ID NO:6, SEQ ID NO:7, SEQ ID NO:8, SEQ ID NO:16, SEQ ID NO:18, SEQ ID NO:26, SEQ ID NO:27, SEQ ID NO:28, SEQ ID NO:37, SEQ ID NO:46, SEQ ID NO:47, and/or SEQ ID NO:48. The antibody or antigen binding fragment can be a monoclonal antibody, a chimeric antibody, a humanized antibody, a synthetic antibody, a single chain antibody, a diabody, or a CDR-grafted antibody. The heavy chain and/or light chain CDRs can include variant CDRs with one or more amino acid substitutions, additions, or deletions, such as from 1 to 3, 1 to 4, 1 to 5, 1 to 6, 1 to 7, 1 to 8, 1 to 9, 1 to 10, or 1 to 11 amino acid substitutions, additions and/or deletions. The substitutions, additions, and/or deletions can occur anywhere within the amino acid sequence. The variant CDRs can be at least about 60%, 65%, 70%, 75%, 80%, 85%, 90%, 95%, 98%, 99%, 99.5% or more identical to a CDR amino acid sequence disclosed herein. The CDRs or variant CDRs are capable of binding human SAS1B, and/or an epitope of human SAS1B, such as human SAS1B amino acid residues 34-53 (GTSFPDGLTPEGTQASGDK) or human SAS1B amino acid residues 64-86 (LEETPESSFLIEGDIIRPSPFRL).

Heavy chain CDRs can be combined with appropriate variable regions of an antibody light chain. Light chain CDRs can be combined with appropriate variable regions of an antibody heavy chain. Alternatively, light chain CDRs may be used independently of the heavy chains and vice versa. The CDRs are substituted for the corresponding CDR1, CDR2, and CDR3, between the framework of a desired light chain or heavy chain variable region.

A polypeptide variant or variant CDR differs by about, for example, up to 1, 2, 3, 4, 5, 6, 7, 8, 9, 10, 20, 30, 40, 50, 60 or more amino acid residues (e.g., amino acid additions, substitutions or deletions) from a polypeptide sequence provided in this disclosure. Where this comparison requires alignment, the sequences are aligned for maximum homology. The site of variation can occur anywhere in the polypeptide. In one embodiment of the present disclosure a variant polypeptide has activity substantially similar to a polypeptide sequence provided in this disclosure. Activity substantially similar means that when the polypeptide is used to construct an antibody, the antibody has the same or substantially the same activity as antibody OV115, OV119, OV121, OV123, and/or OV124.

Methods of introducing a mutation into an amino acid sequence are well known to those skilled in the art. See, e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989). Mutations can also be introduced using commercially available kits such as “QuikChange™ Site-Directed Mutagenesis Kit” (Stratagene). The generation of a functionally active variant polypeptide by replacing an amino acid that does not influence the function of a polypeptide can be accomplished by one skilled in the art.

The variant polypeptides can have conservative amino acid substitutions at one or more predicted non-essential amino acid residues. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his.

A variant polypeptide can also be isolated using a hybridization technique. Briefly, DNA having a high homology to the whole or part of a nucleic acid sequence provided herein or a nucleic acid molecule encoding a polypeptide provided herein is used to prepare a polypeptide. Therefore, a polypeptide of the present disclosure also includes polypeptides that are variants of antibody OV115, OV119, OV121, OV123, and/or OV124, and polypeptides that are encoded by a nucleic acid molecule that hybridizes under high stringency with a nucleic acid molecule provided herein or a complement thereof. One of skill in the art can easily determine nucleic acid sequences that encode polypeptides of the present disclosure using readily available codon tables. As such, these nucleic acid sequences are not presented herein.

As used herein, percent identity of two amino acid sequences (or of two nucleic acid sequences) is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al. (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the present disclosure.

Identity or identical means amino acid sequence (or nucleic acid sequence) similarity and has an art recognized meaning. Sequences with identity share identical or similar amino acids (or nucleic acids). Sequence identity is the percentage of amino acids identical to those in the antibody's original amino acid sequence, determined after the sequences are aligned and gaps are appropriately introduced to maximize the sequence identity as necessary. Thus, a candidate sequence sharing 85% amino acid sequence identity with a reference sequence requires that, following alignment of the candidate sequence with the reference sequence, 85% of the amino acids in the candidate sequence are identical to the corresponding amino acids in the reference sequence, and/or constitute conservative amino acid changes.

Antibodies of the present disclosure can comprise CDRs of OV115, OV119, OV121, OV123, and/or OV124 antibodies or variant antibodies comprising one or more variant CDRs. These variant antibodies can have an activity equivalent (e.g., binding to human SAS1B with the same or substantially similar Kd as an antibody described herein) to that of OV115, OV119, OV121, OV123, and/or OV124. Antibody variants retain substantially the same functional activity of OV115, OV119, OV121, OV123, and/or OV124 antibodies. Naturally-occurring functionally active variant antibodies such as allelic variants and species variants and non-naturally occurring functionally active variants are included in the present disclosure and can be produced by, for example, mutagenesis techniques or by direct synthesis. Antibody variants are encoded by variant polypeptides and variant CDRs of OV115, OV119, OV121, OV123, and/or OV124.

For example, antibodies of the present disclosure can comprise CDRs represented by variable heavy and/or variable light chain CDRs. The heavy chain CDRs can include amino acid sequences represented by SEQ ID NO:1, SEQ ID NO:2, SEQ ID NO:3, SEQ ID NO:11, SEQ ID NO:12, SEQ ID NO:13, SEQ ID NO:21, SEQ ID NO:22, SEQ ID NO:23, SEQ ID NO:31, SEQ ID NO:32, SEQ ID NO:33, SEQ ID NO:41, SEQ ID NO:42, and/or SEQ ID NO:43. The light chain CDRs can include amino acid sequences represented by SEQ ID NO:6, SEQ ID NO:7, SEQ ID NO:8, SEQ ID NO:16, SEQ ID NO:18, SEQ ID NO:26, SEQ ID NO:27, SEQ ID NO:28, SEQ ID NO:37, SEQ ID NO:46, SEQ ID NO:47, and/or SEQ ID NO:48. The antibodies of the present disclosure include antibodies that vary from OV115, OV119, OV121, OV123, and/or OV124 with respect to light chain or heavy chain CDR sequence, other variable regions, and/or light chain or heavy chain constant regions. In particular, antibodies of the present disclosure can include humanized antibodies having any combination of the heavy or light chain CDRs described above. The antibodies can have polypeptide sequences that are entirely human, with the exception of the complementary determining regions (CDRs), which can include heavy and/or light chain CDRs from OV115, OV119, OV121, OV123, and/or OV124.

The present disclosure also includes polypeptide variants or CDR variants of any amino acid sequence provided herein. Polypeptide variants or CDR variants can comprise one or more amino acid substitutions, additions and/or deletions, such as from 1 to 3, 1 to 4, 1 to 5, 1 to 6, 1 to 7, 1 to 8, 1 to 9, 1 to 10, or 1 to 11 amino acid substitutions, additions and/or deletions. In one embodiment, a variant polypeptide or variant CDR includes an amino acid sequence at least about 75% identical to an amino acid sequence provided herein. In one embodiment, the variant polypeptide or CDR is at least about 75%, 80%, 85%, 90%, 95%, 98%, 99%, 99.5% or more identical to a polypeptide sequence provided herein. Variant polypeptides or variant CDRs encode a variant antibody, which is an antibody comprising an amino acid sequence provided herein in which one or more amino acid residues have been added, substituted or deleted. For example, the variable region of an antibody can be modified to improve its biological properties, such as antigen binding. Such modifications can be achieved by e.g., site-directed mutagenesis, PCR-based mutagenesis, cassette mutagenesis. Variant antibodies comprise an amino acid sequence which is at least about 75%, 80%, 85%, 90%, 95%, 98%, 99%, 99.5% or more identical to the amino acid sequence of a heavy or light chain variable region of OV115, OV119, OV121, OV123, and/or OV124. In one embodiment of the present disclosure, a variant antibody retains the same function of a OV115, OV119, OV121, OV123, and/or OV124 antibody (e.g., binds human SAS1B, in particular the propeptide region upstream of the cleavage site of human SAS1B such as amino acid residues 34-53 (GTSFPDGLTPEGTQASGDK) and 64-86 (LEETPESSFLIEGDIIRPSPFRL) at the same or substantially similar Kd as an antibody produced by the hybridomas identified in the Examples, e.g. within about 1, 2, 3, 4, 5, 6, 7, 8, 9, 10, 15, or 20% of the Kd of OV115, OV119, OV121, OV123, and/or OV124). In another embodiment of the present disclosure, a variant antibody may have a function that is somewhat altered from an OV115, OV119, OV121, OV123, or OV124 antibody (e.g., binding human SAS1B with a Kd that is higher or lower than an OV115, OV119, OV121, OV123, or OV124 antibody).

Polypeptide sequences can be modified, for example, by synthesizing multiple polynucleotides encoding the amino acid sequence of a variable region, and preparing nucleic acids encoding the variable region by PCR using the polynucleotides. Antibodies that comprise one or more CDRs can be prepared by inserting the polynucleotide into an appropriate expression vector and expressing the polynucleotide. For example, polynucleotides can be synthesized using mixed nucleotides to prepare a DNA library that encodes a variety of antibodies comprising CDRs with various amino acids introduced at certain positions. An antibody can be isolated by selecting from the library a clone encoding an antibody that binds to human SAS1B with a Kd that is the same or substantially similar to the Kd of OV115, OV119, OV121, OV123, and/or OV124.

A polypeptide or antibody of the present disclosure can be covalently or non-covalently linked to an amino acid sequence to which the polypeptide or antibody is not normally associated with in nature. Additionally, a polypeptide or antibody of the present disclosure can be covalently or non-covalently linked to compounds or molecules other than amino acids. For example, a polypeptide or antibody can be linked to an indicator reagent, an amino acid spacer, an amino acid linker, a signal sequence, a stop transfer sequence, a transmembrane domain, a protein purification ligand, or a combination thereof. In one embodiment of the present disclosure a protein purification ligand can be one or more C amino acid residues at, for example, the amino terminus or carboxy terminus of a polypeptide of the present disclosure. An amino acid spacer is a sequence of amino acids that are not usually associated with a polypeptide or antibody of the present disclosure in nature. An amino acid spacer can comprise about 1, 5, 10, 20, 100, or 1,000 amino acids.

If desired, a polypeptide can be a fusion protein, which can also contain other amino acid sequences, such as amino acid linkers, amino acid spacers, signal sequences, TMR stop transfer sequences, transmembrane domains, as well as ligands useful in protein purification, such as glutathione-S-transferase, histidine tag, and staphylococcal protein A, or combinations thereof. A fusion protein is two or more different amino acid sequences operably linked to each other. A fusion protein construct can be synthesized chemically using organic compound synthesis techniques by joining individual polypeptide fragments together in fixed sequence. A fusion protein construct can also be expressed by a genetically modified host cell (such as E. coli) cultured in vitro, which carries an introduced expression vector bearing specified recombinant DNA sequences encoding the amino acids residues in proper sequence. The heterologous polypeptide can be fused, for example, to the N-terminus or C-terminus of a polypeptide of the present disclosure. A polypeptide of the present disclosure can also comprise homologous amino acid sequences, i.e., other immunoglobulin-derived sequences. More than one polypeptide of the present disclosure can be present in a fusion protein. Fragments of polypeptides of the present disclosure can be present in a fusion protein of the present disclosure. A fusion protein of the present disclosure can comprise, e.g., one or more of amino acid sequences provided herein, fragments thereof, or combinations thereof. Polypeptides of the present disclosure can be in a multimeric form. That is, a polypeptide can comprise two or more copies of an amino acid sequence provided herein or a combination thereof.

In one embodiment of the present disclosure, a polypeptide of the present disclosure is derived from a human, rabbit, mouse, other mammal, or combinations thereof. A polypeptide of the present disclosure can be isolated from cells or tissue sources using standard protein purification techniques. Polypeptides of the present disclosure can also be synthesized chemically or produced by recombinant DNA techniques. For example, a polypeptide of the present disclosure can be synthesized using conventional peptide synthesizers.

A polypeptide of the present disclosure can be produced recombinantly. A polynucleotide encoding a polypeptide of the present disclosure can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system.

Antibodies

The term “antibodies” refers to an intact antibody or an antigen-binding portion or fragment thereof that competes with the intact antibody for antigen binding. The term “antibodies” also includes any type of antibody molecule or specific binding molecule that specifically binds SAS1B. The terms “antigen-binding portion” of an antibody, “antigen-binding fragment” of an antibody, and the like, as used herein, include any naturally occurring, enzymatically obtainable, synthetic, or genetically engineered polypeptide, glycoprotein, or immunoglobulin that specifically binds SAS1B to form a complex.

Antigen-binding fragments of an antibody may be derived, e.g., from full antibody molecules using any suitable standard techniques such as proteolytic digestion or recombinant genetic engineering techniques involving the manipulation and expression of nucleic acids encoding antibody variable and optionally constant domains.

An antibody of the present disclosure can be any isotype including IgG (IgG1, IgG2, IgG2a, Ig2b, IgG3, IgG4), IgM, IgA (IgA1 and IgA2), IgD, and IgE.

A monoclonal antibody is an antibody obtained from a group of substantially homogeneous antibodies. A group of substantially homogeneous antibodies can contain a small amount of mutants or variants. Monoclonal antibodies are highly specific and interact with a single antigenic site. Each monoclonal antibody typically targets a single epitope, while polyclonal antibody populations typically contain various antibodies that target a group of diverse epitopes. Monoclonal antibodies can be produced by many methods including, for example, hybridoma methods (Kohler and Milstein, Nature 256:495, 1975), recombination methods (U.S. Pat. No. 4,816,567), and isolation from phage antibody libraries (Clackson et al., Nature 352:624-628, 1991; Marks et al., J. Mol. Biol. 222:581-597, 1991).

A “humanized antibody or antigen-binding fragment” thereof is an antibody or fragment thereof that has been engineered to comprise one or more human framework regions in the variable region together with non-human (e.g., mouse, rabbit, rat, or hamster) complementarity-determining regions (CDRs) of the heavy and/or light chain. In some embodiments, a humanized antibody comprises sequences that are entirely human except for the CDR regions. Humanized antibodies are typically less immunogenic to humans, relative to non-humanized antibodies, and thus offer therapeutic benefits in certain situations.

One implementation is a humanized antibody or antigen binding fragment having one or more heavy chain or light chain CDRs that binds one or more epitope of human SAS1B. For example, the humanized antibody can bind an epitope such as human SAS1B amino acid residues 34-53 (GTSFPDGLTPEGTQASGDK) or human SAS1B amino acid residues 64-86 (LEETPESSFLIEGDIIRPSPFRL).

Another implementation is a humanized antibody or antigen binding fragment having up to three heavy chain CDRs selected from SEQ ID NO:1, SEQ ID NO:2, SEQ ID NO:3, SEQ ID NO:11, SEQ ID NO:12, SEQ ID NO:13, SEQ ID NO:21, SEQ ID NO:22, SEQ ID NO:23, SEQ ID NO:31, SEQ ID NO:32, SEQ ID NO:33, SEQ ID NO:41, SEQ ID NO:42, and SEQ ID NO:43, and/or up to three light chain CDRs selected from SEQ ID NO:6, SEQ ID NO:7, SEQ ID NO:8, SEQ ID NO:16, SEQ ID NO:18, SEQ ID NO:26, SEQ ID NO:27, SEQ ID NO:28, SEQ ID NO:37, SEQ ID NO:46, SEQ ID NO:47, and SEQ ID NO:48. Another implementation is a humanized antibody or antigen binding fragment with said heavy chain CDRs and/or light chain CDRs, where the CDRs have one or more amino acid substitutions, additions and/or deletions, such as from 1 to 3, 1 to 4, 1 to 5, 1 to 6, 1 to 7, 1 to 8, 1 to 9, 1 to 10, or 1 to 11 amino acid substitutions, additions and/or deletions. (i.e. variant CDRs). The variant CDRs can be at least about 60%, 65%, 75%, 80%, 85%, 90%, 95%, 98%, 99%, 99.5% or more identical to a CDR amino acid sequence disclosed herein.

A “human antibody or antigen binding fragment thereof” is an antibody or antigen binding fragment thereof that contains only human-derived amino acid sequences. For example, a fully human antibody may be produced from a human B-cell or a human hybridoma cell. In additional embodiments, the antibody may be produced from a transgenic animal that contains the locus for a human heavy chain immunoglobulin and a human light chain immunoglobulin, or contains a nucleic acid that encodes the heavy and light chains of a specific human antibody. A human antibody or antigen binding fragment thereof is still considered a “human antibody or antigen binding fragment thereof” even if the framework and/or CDRs of the heavy chain variable domain or light chain variable domain of the antibody isolated or obtained from a human cell, human cell line, or other methodology are mutated (e.g., by amino acid substitution(s), addition(s), and/or deletion(s)) to improve the affinity or other properties of the antibody. In certain embodiments, after the human antibody isolated or obtained from a human cell or human cell line is mutated so that it has at least about 85%, 86%, 87%, 88%, 89%, 90%, 91%, 92%, 93%, 94%, 95%, 96%, 97%, 98%, 99%, or 99.5% identity to the amino acid sequence of the antibody isolated or obtained from a human cell or human cell line. In some embodiments, six, five, four, three, two, or one amino acid substitutions are made in one, two, three, four, five, and/or six of the CDRs. In some embodiments, six, five, four, three, two, or one amino acid substitutions are made in one, two, three, or four framework regions of the heavy chain variable region, one, two, three, or four framework regions of the light chain variable region of the antibody, Fc, hinge region, or combinations thereof. In one embodiment, a human antibody of the present disclosure has an amino acid sequence that is substantially identical to an antibody isolated or obtained from a human cell or human cell line, but is not naturally occurring. The non-naturally occurring human antibody has one or more mutations in the amino acid sequence that do not occur in the variable heavy or light CDR regions, and do not affect the binding or therapeutic characteristics of the human antibody.

Chimeric antibodies or antigen-binding portions thereof have a part of a heavy chain and/or light chain that is derived from a specific species or a specific antibody class or subclass, and the remaining portion of the chain is derived from another species, or another antibody class or subclass. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397.

Chimeric antibodies can be produced using a variety of techniques including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28:489-498 (1991); Studnicka et al., Protein Engineering 7(6):805-814 (1994); Roguska et al., PNAS 96:969-973 (1994)), and chain shuffling (U.S. Pat. No. 5,565,332).

In one embodiment, a chimeric antibody can comprise variable and constant regions of species that are different from each other, for example, an antibody can comprise the heavy chain and light chain variable regions of a non-human mammal such as a mouse or rabbit, and the heavy chain and light chain constant regions of a human. Such an antibody can be obtained by ligating a polynucleotide encoding a variable region of a mouse or rabbit antibody to a polynucleotide encoding a constant region of a human antibody; incorporating the ligated polynucleotides into an expression vector; and introducing the vector into a host cell for production of the antibody. See WO 96/02576. The host cells can be eukaryotic cells, such as mammalian cells, including, e.g., CHO cells, lymphocytes, and myeloma cells. The chimeric antibody can comprise additional amino acid acids that are not included in the CDRs introduced into the recipient antibody, nor in the framework sequences. These amino acids can be introduced to more accurately optimize the antibody's ability to recognize and bind to an antigen. For example, as necessary, amino acids in the framework region of an antibody variable region can be substituted such that the CDR of a reshaped antibody forms an appropriate antigen-binding site. See Sato et al., Cancer Res. (1993) 53:851-856.

Non-limiting examples of antigen-binding fragments of antibodies include: Fab fragments; Fab′ fragments, Fab′-SH fragments, F(ab′)2 fragments; Fd fragments; Fv fragments; single-chain Fv (scFv) molecules; sdAb fragments (nanobodies); Fab-like antibodies (an antigen-binding fragment containing variable regions of a heavy chain and light chain that is equivalent to Fab fragments that are obtained by papain digestion); F(ab′)2-like antibodies (an antigen-binding fragment containing two antigen-binding domains that is equivalent to F(ab′)2 fragments that are obtained by pepsin digestion), multispecific antibodies prepared from antibody fragments, diabody, bispecific antibody, multifunctional antibody, chimeric antibody, humanized antibody, human antibody, murine antibody, rabbit antibody synthetic antibody, CDR-grafted antibody, and minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies), single-chain (Fv)2 (sc(Fv)2); divalent (sc(Fv)2); tetravalent ([sc(Fv)2]2) scFV antibodies, and small modular immunopharmaceuticals (SMIPs), and shark variable IgNAR domains, are also encompassed within the expression “antigen-binding fragment,” as used herein.

An antigen-binding fragment of an antibody will typically comprise at least 1 variable domain. The variable domain may be of any size or amino acid composition and will generally comprise at least 1, 2 or 3 CDRs, which are adjacent to or in frame with 1, 2, 3, or 4 framework sequences, in antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

Antigen-binding fragments can be produced by recombinant DNA techniques or by enzymatic or chemical cleavage of intact antibodies.

One implementation is an antigen-binding fragment having one or more heavy chain or light chain CDRs that binds one or more epitope of human SAS1B. For example, the antigen-binding fragment can bind an epitope such as human SAS1B amino acid residues 34-53 (GTSFPDGLTPEGTQASGDK) or human SAS1B amino acid residues 64-86 (LEETPESSFLIEGDIIRPSPFRL).

In another implementation, antigen-binding fragments are provided that include one or more heavy chain CDRs that can include amino acid sequences represented by SEQ ID NO:1, SEQ ID NO:2, SEQ ID NO:3, SEQ ID NO:11, SEQ ID NO:12, SEQ ID NO:13, SEQ ID NO:21, SEQ ID NO:22, SEQ ID NO:23, SEQ ID NO:31, SEQ ID NO:32, SEQ ID NO:33, SEQ ID NO:41, SEQ ID NO:42, and/or SEQ ID NO:43, and/or one or more light chain CDRs that can include amino acid sequences represented by SEQ ID NO:6, SEQ ID NO:7, SEQ ID NO:8, SEQ ID NO:16, SEQ ID NO:18, SEQ ID NO:26, SEQ ID NO:27, SEQ ID NO:28, SEQ ID NO:37, SEQ ID NO:46, SEQ ID NO:47, and/or SEQ ID NO:48. The antigen-binding fragments can be prepared by methods described herein.

A “diabody” is a bivalent minibody constructed by gene fusion (see, e.g., Holliger et al., Proc. Natl. Acad. Sci. U.S.A, 90:6444 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between about 2 to 12 residues (e.g., about 2, 3, 4, 5, 6, 7, 8, 9, 10, 11, or 12). The linkers of the polypeptides in a diabody are typically too short to allow the VL and VH to bind to each other. Diabody technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

A scFv is a single-chain polypeptide antibody obtained by linking the VH and VL with a linker (see e.g., Huston et al., PNAS USA, 85:5879 (1988); Pluckthun, “The Pharmacology of Monoclonal Antibodies” Vol. 113, Ed Resenburg & Moore, Springer Verlag, New York, pp. 269-315, (1994)). The order of VHs and VLs to be linked is not particularly limited, and they may be arranged in any order. Examples of arrangements include: VH-linker-VL; or VL-linker-VH. The H chain V region and L chain V region in a scFv may be derived from any anti-SAS1B antibody or antigen-binding fragment thereof described herein.

A sc(Fv)2 is a fragment where two VHs and two VLs are linked by a linker to form a single chain (Hudson et al., J. Immunol. Methods, 231:177 (1999)). A sc(Fv)2 molecule can be prepared, for example, by connecting scFvs with a linker. sc(Fv)2 molecules can include antibodies where two VHs and two VLs are arranged in the order of: VH, VL, VH, and VL (VH-linker-VL-linker-VH-linker-VL), beginning from the N terminus of a single-chain polypeptide; however the order of the two VHs and two VLs is not limited to this arrangement, and they may be arranged in any order. Examples of arrangements are listed below:

VL-linker-VH-linker-VH-linker-VL;

VH-linker-VL-linker-VL-linker-VH;

VH-linker-VH-linker-VL-linker-VL;

VL-linker-VL-linker-VH-linker-VH; or

VL-linker-VH-linker-VL-linker-VH.

Three linkers are usually required when four antibody variable regions are linked; the linkers used may be identical or different. There is no limitation on the linkers that link the VH and VL regions of the antibody fragments. In some embodiments, the linker is a peptide linker. Any arbitrary single-chain peptide comprising about three to 25 residues (e.g., 5, 6, 7, 8, 9, 10, 11, 12, 13, 14, 15, 16, 17, 18 amino acids) can be used as a linker. Examples of such peptide linkers include: Ser; Gly Ser; Gly Gly Ser; Ser Gly Gly; Gly Gly Gly Ser; Ser Gly Gly Gly; Gly Gly Gly Gly Ser; Ser Gly Gly Gly Gly; Gly Gly Gly Gly Gly Ser; Ser Gly Gly Gly Gly Gly; Gly Gly Gly Gly Gly Gly Ser; Ser Gly Gly Gly Gly Gly Gly; (Gly Gly Gly Gly Ser)n, wherein n is an integer of one or more; and (Ser Gly Gly Gly Gly)n, wherein n is an integer of one or more.

In certain embodiments, the linker is a synthetic compound linker (chemical cross-linking agent). Examples of cross-linking agents include, for example, N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2 (sulfosuccinimidooxycarbonyloxy)ethyl]sulfone (sulfo-BSOCOES).

Bispecific antibodies are antibodies that have binding specificities for at least two different epitopes. Exemplary bispecific antibodies may bind to two different epitopes of the SAS1B protein. Other such antibodies may combine an SAS1B binding site with a binding site for another protein. Bispecific antibodies can be prepared as full length antibodies or low molecular weight forms thereof (e.g., F(ab′)2 bispecific antibodies, sc(Fv)2 bispecific antibodies, diabody bispecific antibodies). Full length bispecific antibodies can be produced based on the co-expression of two immunoglobulin heavy chain-light chain pairs, where the two chains have different specificities (Millstein et al., Nature, 305:537-539 (1983)). Alternatively, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields. Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

Antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises an Fc region or a hinge region. A multivalent antibody can comprise about 3, 4, 5, 6, 7, 8, or more antigen binding sites. The multivalent antibody optionally comprises at least one, two, three or more polypeptide chains, wherein the polypeptide chain(s) comprise two or more variable domains. For instance, the polypeptide chain(s) may comprise VDI-(XI)n-VD2-(X2)n-Fc, wherein VD1 is a first variable domain, VD2 is a second variable domain, Fc is a polypeptide chain of an Fc region, XI and X2 represent an amino acid or peptide spacer, and n is 0 or 1.

Epitopes

One embodiment of the present disclosure provides binding molecules (e.g., antibodies or antigen-binding fragments) that specifically bind to human SAS1B. In one embodiment, the antibodies or antigen-binding fragments thereof specifically bind to an epitope in particular in the propeptide region of human SAS1B. In a specific embodiment, the antibodies or antigen-binding fragments thereof specifically bind to an epitope of human SAS1B in the propeptide region such as amino acid residues 34-53 (GTSFPDGLTPEGTQASGDK) and 64-86 (LEETPESSFLIEGDIIRPSPFRL) or combinations thereof. The antibody or antigen-binding portion thereof may bind to conformational epitope which comprises 2 or more of these regions.

An antibody or fragment thereof of the present disclosure binds to an epitope that overlaps with or is the same (i.e., a substantially identical epitope) as any of the monoclonal antibodies OV115, OV119, OV121, OV123, and/or OV124. An antibody that binds to an epitope substantially identical to an epitope of human SAS1B to which a monoclonal antibody such as OV115, OV119, OV121, OV123, and/or OV124 binds, can be obtained by analyzing epitopes of the monoclonal antibodies OV115, OV119, OV121, OV123, and/or OV124 using well known epitope mapping methods. Competitive assays can be used to determine if two antibodies bind to a substantially identical epitope of SAS1B. Where the binding of a first anti-SAS1B antibody with SAS1B is competitively inhibited by a second anti-SAS1B antibody, the first antibody and the second antibody can be considered to bind to a substantially identical epitope on SAS1B. Competitively inhibits means that an antibody or antigen-binding fragment thereof can specifically bind an epitope that a monoclonal antibody produced by a hybridoma cell line shown in the Examples, such as OV115, OV119, OV121, OV123, and/or OV124, is directed to, using conventional reciprocal antibody competition assays. See e.g., Belanger et al. (1973), Clinica Chimica Acta 48:15.

Antibodies that competitively inhibit binding of one or more of OV115, OV119, OV121, OV123, and/or OV124 or antigen-binding fragments thereof, reduce the binding of one or more of OV115, OV119, OV121, OV123, and/or OV124 or antigen binding fragments thereof to a SAS1B polypeptide (e.g., a full-length SAS1B polypeptide or the propeptide region such as amino acid residues 34-53 (GTSFPDGLTPEGTQASGDK) and 64-86 (LEETPESSFLIEGDIIRPSPFRL)) or to cancer cells by about 40%, 50%, 75%, 90% or 100% in any type of competitive inhibition assay (see, e.g., Harlow and Lane, Antibodies, A Laboratory Manual, Cold Spring Harbor Publications, NY (1988)) are also antibodies of the present disclosure. Antibodies and antigen-binding fragments thereof can inhibit the binding OV115, OV119, OV121, OV123, and/or OV124 to human SAS1B.

Therefore, the present disclosure comprises antibodies that bind to an epitope that is substantially identical to or the same as an epitope of SAS1B to which an antibody produced by a hybridoma identified in the Examples (e.g. OV115, OV119, OV121, OV123, and/or OV124) binds, and that can also comprise the activity of binding to cancer cells (e.g., uterine or pancreatic cancer cells), or binding to oocytes, or binding to SAS1B or fragments thereof (e.g. amino acid residues 34-53 (GTSFPDGLTPEGTQASGDK) and/or 64-86 (LEETPESSFLIEGDIIRPSPFRL)).

Amount of Binding

Antibodies of the present disclosure specifically bind SAS1B (e.g. human SAS1B). “Specifically binds” means that the antibody recognizes and binds to SAS1B with greater affinity than to other, non-specific molecules that are not SAS1B. For example, an antibody raised against an antigen (polypeptide) to which it binds more efficiently than to a non-specific antigen (e.g., a protein that is not related to or homologous to SAS1B) can be described as specifically binding to the antigen. Binding specificity can be tested using, for example, an enzyme-linked immunosorbant assay (ELISA), a radioimmunoassay (RIA), or a western blot assay using methodology well known in the art.

Antibodies of the present disclosure, antigen-binding fragments thereof, or variants thereof can specifically bind SAS1B with a wide range of disassociation constants (Kd). For example, an antibody can bind human SAS1B with a Kd equal to or less than about 10⁻⁷ M, such as but not limited to, 0.1-9.9×10⁻⁵, 10⁻⁶, 10⁻⁷, 10⁻¹, 10⁻⁹, 10⁻¹⁰, 10⁻¹¹, 10⁻¹², 10⁻¹³, 10⁻¹⁴, 10⁻¹⁵ or any range or value therein, as determined by e.g., surface plasmon resonance or the Kinexa method. The present disclosure encompasses antibodies that bind human SAS1B polypeptides with a disassociation constant or Kd that is within any one of the ranges that are between each of the individual recited values. An antibody has the same or substantially identical activity as antibodies produced by the hybridomas identified in the Examples (e.g. OV115, OV119, OV121, OV123, and/or OV124) when the Kd for binding to SAS1B is within about 0.1, 0.2, 0.3, 0.4, 0.5, 1, 2, 3, 4, 5, 6, 7, 8, 9, 10, 15, or 20% (or any range or particular value between 0.1 and 20%) of the Kd for binding to SAS1B of an antibody such as a OV115, OV119, OV121, OV123, and/or OV124.

Antibodies of the present disclosure, antigen-binding fragments thereof or variants thereof can specifically bind human SAS1B polypeptides with an off rate (Koff) of less than or equal to 01.−9.9×10⁻³ sec⁻¹, 10⁻⁴ sec⁻¹, 10⁻⁵ sec⁻¹, 10⁻⁶ sec⁻¹, 10⁻⁷ sec⁻¹. The present disclosure encompasses antibodies that specifically bind SAS1B polypeptides with an off rate that is within any one of the ranges that are between each of the individual recited values. An antibody has the same or substantially identical activity as antibodies produced by the hybridomas identified in the Examples (such as OV115, OV119, OV121, OV123, and/or OV124) when the Koff for binding to SAS1B is within about 0.1, 0.2, 0.3, 0.4, 0.5, 1, 2, 3, 4, 5, 6, 7, 8, 9, 10, 15, or 20% (or any range or particular value between 0.1 and 20%) of the Koff for binding to SAS1B of an antibody produced by the hybridomas. identified in the Examples (such as OV115, OV119, OV121, OV123, and/or OV124).

Antibodies of the present disclosure, antigen-binding fragments thereof, or variants thereof can specifically bind SAS1B polypeptides with an on rate (Kon) greater than or equal to 0.1-9.9×10³ M⁻¹ sec⁻¹, 10⁴ M⁻¹ sec⁻¹, 10⁵ M⁻¹ sec⁻¹, 10⁶ M⁻¹ sec⁻¹, 10⁷ M⁻¹sec⁻¹, 10⁸ M⁻¹ sec⁻¹. The present disclosure encompasses antibodies that bind SAS1B polypeptides with an on rate that is within any one of the ranges that are between each of the individual recited values. An antibody has the same or substantially identical activity as antibodies produced by the hybridomas identified in the Examples (such as OV115, OV119, OV121, OV123, and/or OV124) when the Kon for binding to SAS1B (e.g. the propeptide region such as amino acid residues 34-53 (GTSFPDGLTPEGTQASGDK) and 64-86 (LEETPESSFLIEGDIIRPSPFRL)) is within about 0.1, 0.2, 0.3, 0.4, 0.5, 1, 2, 3, 4, 5, 6, 7, 8, 9, 10, 15, or 20% (or any range or particular value between 0.1 and 20%) of the Kon for binding to SAS1B (e.g. the propeptide region such as amino acid residues 34-53 (GTSFPDGLTPEGTQASGDK) and 64-86 (LEETPESSFLIEGDIIRPSPFRL)) of an antibody produced by the hybridomas identified in the Examples (e.g. OV115, OV119, OV121, OV123, and/or OV124).

Methods of Making Antibodies

Antibodies of the present disclosure can be produced using methods known to those of skill in the art. For example, an SAS1B antigen or a fragment thereof (e.g., the propeptide region such as amino acid residues 34-53 (GTSFPDGLTPEGTQASGDK) and 64-86 (LEETPESSFLIEGDIIRPSPFRL)) can be used to immunize animals. SAS1B or a fragment thereof can be conjugated to a carrier protein and/or administered to the animals with an adjuvant. An SAS1B antigen can comprise one or more epitopes (i.e., antigenic determinants). An epitope can be a linear epitope, sequential epitope or a conformational epitope. Epitopes within a polypeptide of the present disclosure can be identified by several methods. See, e.g., U.S. Pat. No. 4,554,101; Jameson & Wolf, CABIOS 4:181-186 (1988). For example, SAS1B can be isolated and screened. A series of short peptides, which together span the entire SAS1B polypeptide sequence, can be prepared by proteolytic cleavage. By starting with, for example, 100-mer polypeptide fragments, each fragment can be tested for the presence of epitopes recognized in an ELISA. For example, in an ELISA assay an SAS1B antigen, such as a 100-mer polypeptide fragment, is attached to a solid support, such as the wells of a plastic multi-well plate. A population of antibodies are labeled, added to the solid support and allowed to bind to the unlabeled antigen, under conditions where non-specific absorption is blocked, and any unbound antibody and other proteins are washed away. Antibody binding is detected by, for example, a reaction that converts a colorless substrate into a colored reaction product. Progressively smaller and overlapping fragments can then be tested from an identified 100-mer to map the epitope of interest.

Methods for preparing monoclonal antibodies from hybridomas are well known to those of skill in the art and include, e.g., standard cell culture methods and ascites production methods. Recombinant antibodies or fragments thereof produced by gene engineering can be made using the polynucleotide sequences of the present disclosure. Genes encoding antibodies or fragments thereof can be isolated from hybridomas of the present disclosure or other hybridomas. The genes can be inserted into an appropriate vector and introduced into a host cell. See, e.g., Borrebaeck & Larrick, Therapeutic Monoclonal Antibodies, Macmillan Publ. Ltd, 1990.

Antibodies can be produced using immunospot array assay on a chip (ISAAC) to obtain an antibody gene by screening single B cells, which secrete a specific monoclonal antibody, within several weeks (Jin et al., 2009 Nat. Med. 15, 1088-1092). Whole antibodies can also be made using PCR primers having VH or VL nucleotide sequences, a restriction site, and a flanking sequence to protect the restriction site to amplify the VH or VL sequences in scFv clones. Using well known cloning techniques, the PCR amplified VH domains can be cloned into vectors expressing a VH constant region, e.g., a human, rabbit or mouse constant region, and the PCR amplified VL domains can be cloned into vectors expressing a VL constant region, e.g., a human VL constant region or rabbit or murine light constant regions. The vectors for expressing the VH or VL domains can comprise, e.g., a promoter suitable to direct expression of the heavy and light chains in the chosen expression system, a secretion signal, a cloning site for the immunoglobulin variable domain, immunoglobulin constant domains, and a selection marker. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The nucleic acid sequences for human, rabbit, and mouse IgG constant regions have been cloned and sequenced and can be used to construct antibodies of the present disclosure.

Human antibodies can be made by sensitizing human lymphocytes with antigens of interest or cells expressing antigens of interest in vitro; and fusing the sensitized lymphocytes with human myeloma cells. Alternatively, a human antibody can be made by using an antigen to immunize a transgenic animal that comprises a partial or entire repertoire of human antibody genes. See Green et al., Nature Genetics 7:13-21 (1994); Mandez et al., Nature Genetics 15:146-156 (1997); Lonberg et al., Nature 368:856-859 (1994); WO 93/12227; WO 92/03918; WO 94/02602, WO 94/25585, WO 96/34096, and WO 96/33735).

Human antibodies can also be made by panning with a human antibody library. For example, the variable region of a human antibody is expressed as a single chain antibody (scFv) on the surface of a phage, using phage display method, and phages that bind to the antigen are selected. By analyzing the polynucleotides of selected phages, the polynucleotides encoding the variable regions of human antibodies that bind to the antigen can be determined. If the polynucleotide sequences of scFvs that bind to the antigen are identified, appropriate expression vectors comprising these sequences can be constructed, and then introduced into appropriate hosts and expressed to obtain human antibodies. See WO 92/01047, WO 92/20791, WO 93/06213, WO 93/11236, WO 93/19172, WO 95/01438, and WO 95/15388. These same human antibody production methods can be used to make mouse or rabbit antibodies.

Antibodies and fragments thereof can be purified by any method, including, e.g., protein A-Sepharose methods, hydroxyapatite chromatography, salting-out methods with sulfate, ion exchange chromatography, affinity chromatography, filtration, ultrafiltration, dialysis, preparative polyacrylamide gel electrophoresis, isoelectrofocusing or combinations thereof.

Antibodies can be dried or lyophilized (“freeze-dried) for more ready formulation into a desired vehicle/carrier where appropriate and for increased shelf-life.

Conjugates

Antibodies of the present disclosure can be covalently attached to other molecules such that covalent attachment does not affect the ability of the antibody to bind to SAS1B or cells expressing SAS1B. For example, antibodies can be modified by, e.g., glycosylation, acetylation, pegylation, phosphorylation, amidation, derivatization by known protecting/blocking groups (e.g., methyl group, ethyl group, carbohydrate group), proteolytic cleavage, linkage to a cellular ligand or other protein.

Conjugated antibodies can be bound to various molecules including, for example, polymers, hyaluronic acid, fluorescent substances, luminescent substances, haptens, enzymes, metal chelates, cytotoxic agents, radionuclides, and drugs.

An anti-SAS1B antibody or antigen-binding fragment thereof can be modified with a moiety that improves its binding, stabilization and/or retention in circulation, e.g., in blood, serum, or other tissues, e.g., by at least 1.5, 2, 5, 10, or 50 fold. For example, the anti-SAS1B antibody or antigen-binding fragment thereof can be associated with (e.g., conjugated to) a polymer, e.g., a substantially non-antigenic polymer, such as a polyalkylene oxide, a polyethylene oxide, polyethylene glycol (PEG), polyethylenimine (PEI) modified with PEG (PEI-PEG), polyglutamic acid (PGA) (N-(2-Hydroxypropyl) methacrylamide (HPMA) copolymers. Suitable polymers will vary substantially by weight.

Polymers having molecular number average weights ranging from about 200 to about 35,000 Daltons (or about 1,000 to about 15,000, and 2,000 to about 12,500) can be used. For example, the anti-SAS1B antibody or antigen-binding fragment thereof can be conjugated to a water soluble polymer, a hydrophilic polyvinyl polymer, polyvinylalcohol or polyvinylpyrrolidone. Examples of such polymers include polyalkylene oxide homopolymers such as polyethylene glycol (PEG) (see e.g., Chapman et al., Nature Biotechnology, 17: 780 (1999), or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides. The antibodies or antigen-binding fragments thereof can also be conjugated to small molecules and other chemical moieties. Conjugated antibodies can be prepared by performing chemical modifications on the antibodies or fragments thereof. See e.g., U.S. Pat. Nos. 5,057,313 and 5,156,840.

In one aspect, an antibody or a fragment or homolog thereof of the invention can be conjugated to an imaging agent. In one embodiment, antibody complex comprises an imaging agent selected from the group consisting of a radionuclide, a radiological contrast agent, a paramagnetic ion, a metal, a biological tag, a fluorescent label, a chemiluminescent label, an ultrasound contrast agent and a photoactive agent. In one aspect, the imaging agent is a radionuclide. In one aspect, the radionuclide is selected from the group consisting of ¹¹⁰In, ¹¹¹In, ¹⁷⁷Lu, ¹⁸F, ⁵²Fe, ⁶²Cu, 64Cu, ⁶⁷Cu, ⁶⁷Ga, ⁶⁸Ga, ⁸⁶Y ⁹⁰Y, ⁸⁹Zr, ⁹⁴mTc, ⁹⁴Tc, ⁹⁹mTc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, and other gamma-, beta-, or positron-emitters. In one aspect, the radionuclide is ¹¹¹In.

The invention further provides for use of the monoclonal antibodies described herein for drug delivery and for diagnostics. For example, various agents as described herein can be conjugated to the antibodies. Drugs such as calicheamicin, peptides such as D(KLAKLAK)², and radionuclides such as beta ⁹⁰Y, gamma ¹³¹I, and positron ¹²⁴I emitters can be conjugated to monoclonal antibodies to human SAS1B and used to image lung or other tumors, as radiotherapeutic and chemotherapeutic agents for treatment.

Variants of Antibodies

The constant region of an antibody or antigen-binding fragment thereof can be a human Fc region, e.g., a wild-type Fc region, or an Fc region that includes one or more amino acid substitutions. The constant region can have substitutions that modify the properties of the antibody (e.g., increase or decrease one or more of: Fc receptor binding, antibody glycosylation, the number of cysteine residues, effector cell function, or complement function). Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies can also have mutations that stabilize the disulfide bond between the two heavy chains of an immunoglobulin, such as mutations in the hinge region of IgG4, as disclosed in the art (e.g., Angal et al. (1993) Mol. Immunol. 30: 105-08). See also, e.g., U.S. 2005/0037000.

The amino acid sequence of the heavy chain variable region (VH) or the light chain variable region (VL) in the antibody or antibody fragments can include modifications such as amino acid substitutions, deletions, additions, and/or insertions. For example, the modification may be in one or more of the CDRs of the anti-SAS1B antibody or antigen-binding fragment thereof. In certain embodiments, the modification involves one, two, or three amino acid substitutions in one or more CDRs and/or framework regions of the VH and/or VL domain of the anti-SAS1B antibody. Such substitutions are made to improve the binding, functional activity and/or reduce immunogenicity of the anti-SAS1B antibody. The amino acid substitutions can be conservative amino acid substitutions. In one embodiment, one, two, or three amino acids of the CDRs of the anti-SAS1B antibody or antigen-binding fragment thereof may be deleted or added as long as there is SAS1B binding and/or functional activity when VH and VL are associated.

The amino acid sequences of the CDRs are of primary importance for epitope recognition and antibody binding. Changes may be made to the amino acids that comprise the CDRs without interfering with the ability of the antibody to recognize and bind its cognate epitope. For example, changes that do not affect epitope recognition, yet increase the binding affinity of the antibody for the epitope may be made. Thus, also included in the scope of the present disclosure are improved versions of the disclosed antibodies, which also specifically recognize and bind SAS1B, preferably with increased affinity.

The effects of introducing one or more amino acid changes at various positions in the sequence of an antibody has been studied based on the knowledge of the primary antibody sequence, on its properties such as binding and level of expression. See, e.g., Yang et al., 1995, J. Mol. Biol., 254: 392; Rader et al., 1998, Proc. Natl. Acad. Sci. U.S.A., 95: 8910; Vaughan et al., 1998, Nature Biotechnology, 16:535.

For example, equivalents of a primary antibody have been generated by changing the sequences of the heavy and light chain genes in the CDR1, CDR2, CDR3, or framework regions, using methods such as oligonucleotide-mediated site-directed mutagenesis, cassette mutagenesis, error-prone PCR, DNA shuffling, or mutator-strains of E. coli. See Vaughan et al., 1998, Nature Biotechnology, 16: 535; Adey et al., 1996, Chapter 16, pp. 277-291, in “Phage Display of Peptides and Proteins”, Eds. Kay et al., Academic Press). These methods of altering the sequence of a primary antibody have resulted in improved affinities of the newly generated antibodies. Gram et al., 1992, Proc. Natl. Acad. Sci. U.S.A., 89: 3576; Boder et al., 2000, Proc. Natl. Acad. Sci. U.S.A., 97:10701; Davies & Riechmann, 1996, Immunotechnology, 2:169; Thompson et al., 1996, J. Mol. Biol., 256:77; Short et al., 2002, J. Biol. Chem., 277:16365; Furukawa, et al., 2001, J. Biol. Chem., 276:27622.

Using similar directed strategies of changing one or more amino acid residues of the antibody, the antibody sequences described herein can be used to develop anti-SAS1B antibodies with improved functions, including improved affinity for SAS1B.

The present disclosure also encompasses antibodies, fragments thereof, or variants thereof that have one or more of the same or substantially similar biological characteristics as the antibodies OV115, OV119, OV121, OV123, and/or OV124. Biological characteristics are the in vitro or in vivo activities or properties of the antibodies shown in the Figures including, for example, the ability to bind to SAS1B with a substantially similar Kd, Koff, and/or Kon rate, or ability to bind oocytes.

Antibodies of the present disclosure can be used to generate anti-idiotype antibodies that “mimic” human SAS1B polypeptides using techniques well known to those skilled in the art. See, Greenspan & Bona, FASEB 17:437-444 (1993); Nissinoff, J. Immunol. 147:2429-2438 (1991).

One embodiment of the present disclosure provides mixtures of antibodies, antigen-binding fragments thereof, or variants thereof that bind to SAS1B, wherein the mixture has at least two, three, four, five or more different antibodies of the present disclosure.

The present disclosure also provides for panels of antibodies that have different affinities for SASB1, different specificities for SAS1B, or different dissociation rates. The present disclosure provides panels of at least about 2, 3, 4, 5, 6, 7, 10, 20, 50, 100, 250, 500, 750, or 1,000 antibodies.

In one embodiment of the present disclosure, the antibodies or antigen-binding fragments thereof are not naturally occurring due to one or more amino acid mutations in one or more constant regions or one or more framework regions or other mutations.

Polynucleotides

Polynucleotides of the present disclosure contain less than an entire human, mouse or rabbit genome and can be single- or double-stranded nucleic acids. A polynucleotide can be RNA, DNA, cDNA, genomic DNA, chemically synthesized RNA or DNA or combinations thereof. The polynucleotides can be purified free of other components, such as proteins, lipids and other polynucleotides. For example, the polynucleotide can be 50%, 75%, 90%, 95%, 96%, 97%, 98%, 99%, or 100% pure by dry weight. Purity can be measured by a method such as column chromatography, polyacrylamide gel electrophoresis, or HPLC analysis. The polynucleotides of the present disclosure encode the polypeptides described above. In one embodiment of the present disclosure the polynucleotides encode polypeptides disclosed herein or portions or combinations thereof.

The polynucleotides of the present disclosure encode the polypeptides and antibodies of the present disclosure, as well as fragments thereof. A polynucleotide fragment can be about 9, 18, 21, 27, 30, 33, 39, 48, 51, 75, 100, 120, 130, 140, 150, 200 or more polynucleotides. One of skill in the art can obtain the polynucleotide sequences of the present disclosure using the polypeptide sequences and codon tables known to those of skill in the art. Polynucleotides can contain naturally occurring polynucleotides or sequences that differ from those of any naturally occurring sequences or polynucleotides (e.g., non-naturally occurring polynucleotides). Polynucleotides of the present disclosure can differ from naturally occurring nucleic acids, but still encode naturally occurring amino acids due to the degeneracy of the genetic code. Polynucleotides of the present disclosure can also comprise other heterologous nucleotide sequences, such as sequences coding for linkers, signal sequences, amino acid spacers, heterologous signal sequences, TMR stop transfer sequences, transmembrane domains, or ligands useful in protein purification such as glutathione-S-transferase, histidine tag, and staphylococcal protein A. Polynucleotides of the present disclosure can also comprise other nucleotide sequences.

Methods for introducing polynucleotides of the present disclosure (e.g., vectors comprising the polynucleotides or naked polynucleotides) into cells, either transiently or stably, are well known in the art. For example, transformation methods using standard CaCl₂), MgCl₂, or RbCl methods, protoplast fusion methods or transfection of naked or encapsulated nucleic acids using calcium phosphate precipitation, microinjection, viral infection, and electroporation.

In one embodiment of the present disclosure, a polynucleotide of the present disclosure is derived from a mammal, such as a human. Polynucleotides can also be synthesized in the laboratory, for example, using an automatic synthesizer. An amplification method such as PCR can be used to amplify polynucleotides from either genomic DNA or cDNA encoding the polypeptides. Polynucleotide molecules encoding a variant polypeptide can also be isolated by a gene amplification method such as PCR using a portion of a nucleic acid molecule DNA encoding a polypeptide disclosed herein or fragments thereof as the probe.

Polynucleotides and fragments thereof of the present disclosure can be used, for example, as probes or primers to detect the presence of SAS1B polynucleotides in a sample, such as a biological sample. A biological sample can be, e.g., lymph node or tissue aspirate, serum, lymphocytes, whole blood, cellular suspension, plasma, circulating tumor cells, tumor cells or tissue, ascites fluid, urine, or fluid effusion. The ability of such probes to specifically hybridize to polynucleotide sequences will enable them to be of use in detecting the presence of complementary sequences in a given sample. Polynucleotide probes of the present disclosure can hybridize to complementary sequences in a sample such as a biological sample, for example, lymph tissue. Polynucleotides from the sample can be, for example, subjected to gel electrophoresis or other size separation techniques or can be dot blotted without size separation. The polynucleotide probes are preferably labeled. Suitable labels, and methods for labeling probes are known in the art, and include, for example, radioactive labels incorporated by nick translation or by kinase, biotin, fluorescent probes, and chemiluminescent probes. The polynucleotides from the sample are then treated with the probe under hybridization conditions of suitable stringencies.

The stringency of hybridization conditions for a polynucleotide encoding a variant polypeptide of the present disclosure to a polynucleotide encoding polypeptides with amino acid sequences disclosed herein can be, for example, 10% formamide, 5×SSPE, 1× Denhart's solution, and 1× salmon sperm DNA (low stringency conditions). In one embodiment, the conditions are, 25% formamide, 5×SSPE, 1× Denhart's solution, and 1× salmon sperm DNA (moderate stringency conditions), and in another embodiment, the conditions are, 50% formamide, 5×SSPE, lx Denhart's solution, and 1× salmon sperm DNA (high stringency conditions). However, several factors influence the stringency of hybridization other than the above-described formamide concentration, and one skilled in the art can suitably select these factors to accomplish a similar stringency. See e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989)). That is, a polynucleotide encoding a variant polypeptide of the present disclosure will hybridize to a polynucleotide disclosed herein under low or high or both stringency conditions.

An isolated polynucleotide is a nucleic acid molecule that is not immediately contiguous with one or both of the 5′ and 3′ flanking sequences with which it is normally contiguous when present in a naturally occurring genome. Therefore, an isolated polynucleotide can be, for example, a polynucleotide that is incorporated into a vector, such as a plasmid or viral vector, a polynucleotide that is incorporated into the genome of a heterologous cell (or the genome of a homologous cell, but at a site different from that where it naturally occurs); and a polynucleotide that exists as a separate molecule such as a polynucleotide produced by PCR amplification, chemically synthesis, restriction enzyme digestion, or in vitro transcription. An isolated polynucleotide is also a nucleic acid molecule, such as a recombinant nucleic acid molecule that forms part of hybrid polynucleotide encoding additional polypeptide sequences that can be used for example, in the production of a fusion protein.

A polynucleotide can also comprise one or more expression control sequences such as promoters or enhancers, for example. A polynucleotide of the present disclosure can be present in a vector, such as, for example, an expression vector. If desired, polynucleotides can be cloned into an expression vector comprising, for example, promoters, enhancers, or other expression control sequences that drive expression of the polynucleotides of the present disclosure in host cells. The polynucleotides can be operably linked to the expression control sequences.

Methods of Detection

One embodiment of the present disclosure provides methods of detecting SAS1B polypeptides in a sample. The methods comprise contacting the sample suspected of containing SAS1B polypeptides with an antibody or antigen binding portion thereof of the present disclosure (e.g. OV115, OV119, OV121, OV123, and/or OV124) to form SAS1B/antibody complexes. The presence of the SAS1B/antibody complexes are detected, thereby detecting the presence of the SAS1B polypeptides.

Another embodiment of the present disclosure provides a method of detection of SAS1B-positive cells (i.e., cells that express SAS1B on their surface) in a test sample comprising contacting one or more antibodies or antigen-binding portions thereof with the test sample under conditions that allow SAS1B-positive cell/antibody complexes to form. The cells can be permeabilized or cell lysates. The SAS1B positive cell/antibody complexes are then detected. The detection of SAS1B positive cell/antibody complexes is an indication that SAS1B cells are present in the test sample. The test sample can be, e.g., lymph node or tissue aspirate, serum, whole blood, cellular suspension, lymphocytes, plasma, circulating tumor cells, tumor cells or tissue, ascites fluid, urine, or fluid effusion. Polypeptide/antibody or SAS1B-positive cell/antibody complexes can be detected by any method known in the art, enzyme-linked immunosorbent assay (ELISA), multiplex fluorescent immunoassay (MFI or MFIA), radioimmunoassay (RIA), sandwich assay, western blotting, immunoblotting analysis, an immunohistochemistry method, immunofluorescence assay, fluorescence-activated cell sorting (FACS) or a combination thereof.

An immunoassay for SAS1B can utilize one antibody or several different antibodies. Immunoassay protocols can be based upon, for example, competition, direct reaction, or sandwich type assays using, for example, labeled antibody. Antibodies of the present disclosure can be labeled with any type of label known in the art, including, for example, fluorescent, chemiluminescent, radioactive, enzyme, colloidal metal, radioisotope and bioluminescent labels.

Antibodies of the present disclosure or antigen-binding portions thereof can be bound to a support and used to detect the presence of SAS1B. Supports include, for example, glass, polystyrene, polypropylene, polyethylene, dextran, nylon, amylases, natural and modified celluloses, polyacrylamides, agaroses and magletite.

Antibodies of the present disclosure can be used in a method of the diagnosis of a hyperproliferative disorder by obtaining a test sample from, e.g., a human or animal suspected of having a hyperproliferative disorder. The test sample is contacted with antibodies or antigen-binding portions thereof of the present disclosure under conditions enabling the formation of antibody-antigen complexes (i.e., immunocomplexes). One of skill in the art is aware of conditions that enable and are appropriate for formation of antigen/antibody complexes. The amount of antibody-antigen complexes (including, for example, a complex of an antibody or antigen-binding portion thereof and a cell expressing SAS1B on its surface) can be determined by methodology known in the art. A level that is higher than that formed in a control sample indicates the presence of a hyperproliferative disorder. A control sample is a sample that does not comprise any SAS1B polypeptides, SAS1B-positive cells, or antibodies specific for SAS1B. The amount of antibody/antigen complexes or antibodies bound to SAS1B-positive cells or cell lysates can be determined by methods known in the art.

A hyperproliferative disorder can be a neoplastic disorder (e.g., breast cancer, ovarian cancer, colorectal cancer, liver cancer, uterine cancer, pancreatic cancer, lung cancer, etc.) or a hematologic malignancy (e.g., leukemia, etc.).

In one embodiment of the present disclosure, a hyperproliferative disorder can be detected in a subject. A biological sample is obtained from the subject. One or more antibodies or antigen-binding portions thereof of the present disclosure are contacted with the biological sample under conditions that allow SAS1B polypeptide/antibody complexes (including, for example, a complex of an antibody or antigen-binding portion thereof and a cell expressing SAS1B on its surface) to form. The SAS1B polypeptide/antibody complexes are detected. The detection of the SAS1B polypeptide/antibody complexes is an indication that the mammal has a hyperproliferative disorder. The lack of detection of the polypeptide/antibody complexes is an indication that the mammal does not have a hyperproliferative disorder.

In one embodiment of the present disclosure, the SAS1B polypeptide/antibody complex is detected when an indicator reagent, such as an enzyme conjugate, which is bound to the antibody, catalyzes a detectable reaction. Optionally, an indicator reagent comprising a signal generating compound can be applied to the polypeptide/antibody complex under conditions that allow formation of a polypeptide/antibody/indicator complex. The polypeptide/antibody/indicator complex is detected. Optionally, the polypeptide or antibody can be labeled with an indicator reagent prior to the formation of a polypeptide/antibody complex. The method can optionally comprise a positive or negative control.

In one embodiment of the present disclosure, one or more antibodies of the present disclosure are attached to a solid phase or substrate. A test sample potentially comprising a polypeptide of the present disclosure is added to the substrate. One or more antibodies that specifically bind SAS1B are added. The antibodies can be the same antibodies used on the solid phase or can be from a different source or species and can be linked to an indicator reagent, such as an enzyme conjugate. Wash steps can be performed prior to each addition. A chromophore or enzyme substrate is added and color is allowed to develop. The color reaction is stopped and the color can be quantified using, for example, a spectrophotometer.

Assays of the present disclosure include, but are not limited to those based on competition, direct reaction or sandwich-type assays, including, but not limited to enzyme linked immunosorbent assay (ELISA), multiplex fluorescent immunoassay (MFI or MFIA) western blot, IFA, radioimmunoassay (RIA), western blot, hemagglutination (HA), fluorescence polarization immunoassay (FPIA), fluorescence-activated cell sorting (FACS), and microtiter plate assays (any assay done in one or more wells of a microtiter plate).

Assays can use solid phases or substrates or can be performed by immunoprecipitation or any other methods that do not utilize solid phases. Where a solid phase or substrate is used, one or more antibodies or antigen-binding portions thereof of the present disclosure are directly or indirectly attached to a solid support or a substrate such as a microtiter well, magnetic bead, non-magnetic bead, column, matrix, membrane, fibrous mat composed of synthetic or natural fibers (e.g., glass or cellulose-based materials or thermoplastic polymers, such as, polyethylene, polypropylene, or polyester), sintered structure composed of particulate materials (e.g., glass or various thermoplastic polymers), or cast membrane film composed of nitrocellulose, nylon, polysulfone or the like (generally synthetic in nature). All of these substrate materials can be used in suitable shapes, such as films, sheets, or plates, or they may be coated onto or bonded or laminated to appropriate inert carriers, such as paper, glass, plastic films, or fabrics. Suitable methods for immobilizing peptides on solid phases include ionic, hydrophobic, covalent interactions and the like.

In one type of assay format, one or more antibodies or antigen-binding portions thereof can be coated on a solid phase or substrate. A test sample suspected of containing SAS1B polypeptides or SAS1B-positive cells is incubated with an indicator reagent comprising a signal generating compound conjugated to an antibody or antigen-binding antibody fragment specific for SAS1B (indicator reagent composition) for a time and under conditions sufficient to form antigen/antibody complexes of either SAS1B polypeptides of the test sample to the antibodies or antigen-binding fragments thereof of the solid phase or the indicator reagent compound. The reduction in binding of the indicator reagent can be quantitatively measured. A measurable reduction in the signal compared to the signal generated from a confirmed negative SAS1B test sample indicates the presence of SAS1B in the test sample. This type of assay can quantitate the amount of SAS1B in a test sample.

The formation of a polypeptide/antibody complex or a polypeptide/antibody/indicator complex (including, for example, a complex of an antibody or antigen-binding portion thereof and a cell expressing SAS1B on its surface or a complex of an antibody or antigen-binding portion thereof, an indicator reagent, and a cell expressing SAS1B on its surface) can be detected by e.g., radiometric, colorimetric, fluorometric, size-separation, or precipitation methods. Optionally, detection of a polypeptide/antibody complex is by the addition of a secondary antibody that is coupled to an indicator reagent comprising a signal generating compound. Indicator reagents comprising signal generating compounds (labels) associated with a polypeptide/antibody complex can be detected using the methods described above and include chromogenic agents, catalysts such as enzyme conjugates fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors, magnetic particles, and the like. Examples of enzyme conjugates include alkaline phosphatase, horseradish peroxidase, beta-galactosidase, and the like. The selection of a particular label is not critical, but it will be capable of producing a signal either by itself or in conjunction with one or more additional substances.

Formation of the complex can be indicative of the presence of SAS1B-positive cells in a test sample. Therefore, the methods of the present disclosure can be used to diagnose a hyperproliferative disease in a mammal, or determine fertility or lack thereof.

The methods of the present disclosure can also indicate the amount or quantity of SAS1B in a test sample. With many indicator reagents, such as enzyme conjugates, the amount of SAS1B present is proportional to the signal generated. Depending upon the type of test sample, it can be diluted with a suitable buffer reagent, concentrated, or contacted with a solid phase without any manipulation. For example, it usually is preferred to test samples that previously have been diluted, or concentrated specimens, in order to determine the presence and/or amount of SAS1B present.

Vectors and Host Cells

A polypeptide can be expressed in systems, e.g., cultured cells, which result in substantially the same post-translational modifications present as when the polypeptide is expressed in a native cell, or in systems that result in the alteration or omission of post-translational modifications, e.g., glycosylation or cleavage, present when expressed in a native cell.

An expression vector can be, for example, a plasmid, such as pBR322, pUC, or ColE1, or an adenovirus vector, such as an adenovirus Type 2 vector or Type 5 vector. Vectors suitable for use in the present disclosure include, for example, bacterial vectors, mammalian vectors, viral vectors (such as retroviral, adenoviral, adeno-associated viral, herpes virus, simian virus 40 (SV40)) and baculovirus-derived vectors for use in insect cells. Polynucleotides in such vectors are preferably operably linked to a promoter, which is selected based on, e.g., the cell type in which expression is sought.

The expression vector can be transferred to a host cell by conventional techniques and the transfected cells are then cultured by conventional techniques to produce an antibody of the present disclosure. The present disclosure includes host cells containing polynucleotides encoding an antibody of the present disclosure (e.g., whole antibody, a heavy or light chain thereof, or portion thereof, or a single chain antibody of the present disclosure, or a fragment or variant thereof), operably linked to a heterologous promoter. For the expression of entire antibody molecules, vectors encoding both the heavy and light chains may be co-expressed in the host cell for expression of the entire immunoglobulin molecule.

Host cells into which vectors, such as expression vectors, comprising polynucleotides of the present disclosure can be introduced include, for example, prokaryotic cells (e.g., bacterial cells) and eukaryotic cells (e.g., yeast cells; fungal cells; plant cells; insect cells; and mammalian cells). Such host cells are available from a number of different sources that are known to those skilled in the art, e.g., the American Type Culture Collection (ATCC), Manassas, Va. Host cells into which the polynucleotides of the present disclosure have been introduced, as well as their progeny, even if not identical to the parental cells, due to mutations, are included in the present disclosure. Host cells can be transformed with the expression vectors to express the antibodies or antigen-binding fragments thereof. Host cells expressing antibodies or antigen-binding fragments thereof of the present disclosure include cells and hybridomas transformed with a polynucleotide of the present disclosure.

One embodiment of the present disclosure provides methods of producing a recombinant cell that expresses an SAS1B antibody, antigen-binding fragment thereof or portion thereof, comprising transfecting a cell with a vector comprising a polynucleotide of the present disclosure. An SAS1B antibody, or fragment, or portion thereof, can then be produced by expressing the polypeptide in the recombinant host cell.

Isolation and purification of polypeptides produced in the systems described above can be carried out using conventional methods, appropriate for the particular system. For example, preparative chromatography and immunological separations employing antibodies, such as monoclonal or polyclonal antibodies, can be used.

Antibody-Drug Conjugates

Antibodies and antigen-binding fragments thereof of the present disclosure that specifically bind SAS1B can be conjugated to a therapeutic agent or effector molecule to form an “antibody-drug conjugate”. An anti-SAS1B antibody-drug conjugate (ADC) refers to an antibody described herein or an antigen-binding portion thereof, linked to a cytotoxic drug moiety (D) via a linker unit molecule (LU). A therapeutic agent is an agent with a biological activity directed against a particular target molecule or a cell bearing a target molecule. Therapeutic agents can include, for example, various drugs such as vinblastine, daunomycin, cytotoxins such as maytansinoids and maytansinoid analogs, a prodrug, tomaymycin derivatives, taxoids, a leptomycin derivative, CC-1065 and CC-1065 analogs, encapsulating agents (such as liposomes) that contain pharmacological compositions, therapeutic agents, toxins (e.g., ricin, abrin, diphtheria toxin and subunits thereof, botulinum toxins A through F, variants of toxins (see, e.g., U.S. Pat. Nos. 5,079,163 and 4,689,401), Pseudomonas exotoxin (PE) (see e.g., U.S. Pat. No. 5,602,095) and variants thereof (see, e.g. U.S. Pat. Nos. 4,892,827; 5,512,658; 5,602,095; 5,608,039; 5,821,238; and 5,854,044; PCT Publication No. WO 99/51643; Pai et al., Proc. Natl. Acad. Sci. USA 88:3358, 1991; Kondo et al., J. Biol. Chem. 263:9470, 1988; Pastan et al., Biochim. Biophys. Acta 1333:C1-C6, 1997)), radioactive agents such as ¹²⁵, ³²P, ¹⁴C, ³H and ³⁵S and other labels, target moieties and ligands. An effector molecule is a small molecule that selectively binds to a protein and regulates its biological activity.

Drug (D): A “drug” is any substance that has biological or detectable activity, such as a therapeutic agent, a detectable label, a binding agent, and the like, and a prodrug that is metabolized in vivo to an active agent. The terms drug and payload are used interchangeably. In some embodiments, the agent is auristatin, such as auristatin E (also known in the art as a derivative of dolastatin-10), or a derivative thereof. The auristatin can be, for example, an ester formed between auristatin E and keto acid. For example, auristatin E can react with paraacetylbenzoic acid or benzoylvaleric acid to produce AEB and AEVB, respectively. Other typical auristatins include AFP, MMAF, and MMAE. Exemplary auristatin syntheses and structures are described in U.S. Pat. Nos. 6,884,869, 7,098,308, 7,256,257, 7,423,116. U.S. Pat. Nos. 7,498,298, and 7,745,394, each of which is incorporated herein by reference in its entirety for all purposes.

Auristatin has been shown to interfere with microtubule dynamics and nuclear and cellular division and have anti-cancer activity. The auristatin of the present invention binds tubulin and may have a cytotoxic or cytostatic effect on cells or cell lines expressing 5T4. There are many different assays that can be used to determine whether auristatin or the resulting antibody-drug conjugate has a cytostatic or cytotoxic effect on a desired cell or cell line. Are known in the art. Methods for determining whether a compound binds tubulin are known in the art. For example, Muller et al., Anal. See Chem 2006, 78, 4390-4397; Hamel et al., Molecular Pharmacology, 1995 47: 965-976; and Hamel et al., The Journal of Biological Chemistry, 1990 265: 28, 17141-17149. (JP5925875B2)

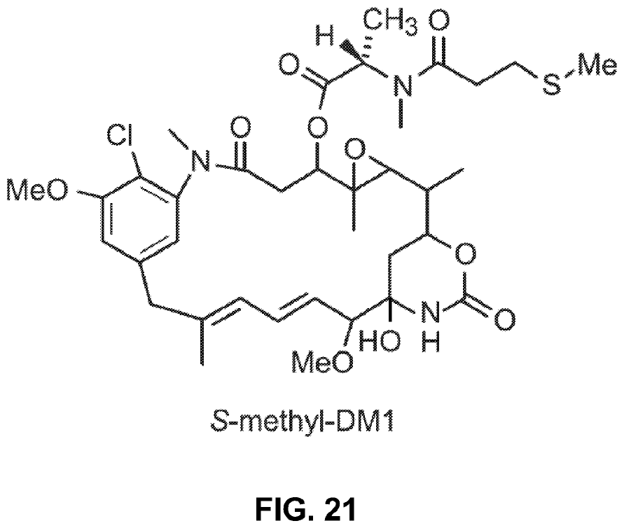

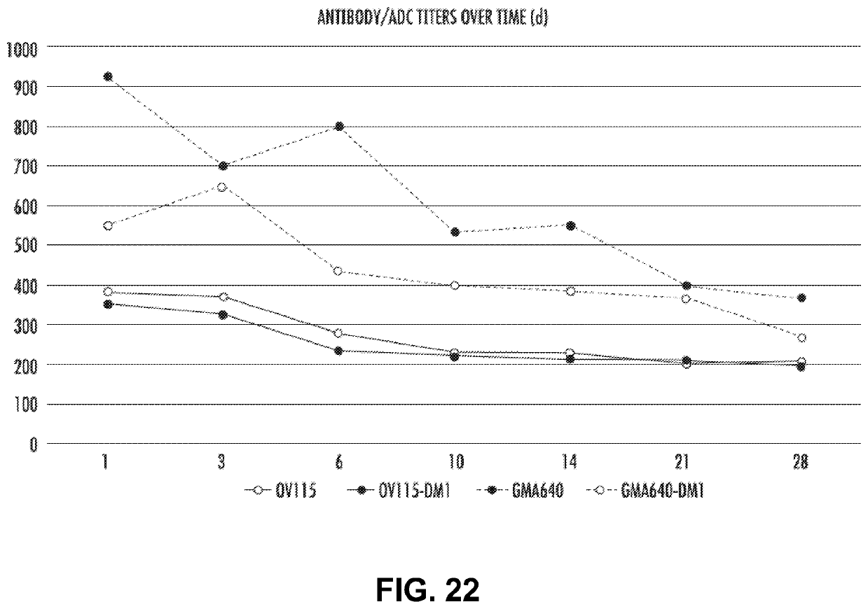

Examples of drugs or payloads are DM1, the chemical structure of which is shown in FIG. 21 (maytansine, N2′-deacetyl-N2′-(3-mercapto-1-oxopropyl)- or N2′-deacetyl-N2′-(3-mercapto-1-oxopropyl)-Maytansine), mc-MMAD (6-maleimidocaproyl-monomethylauristatin-D or N-methyl-L-valyl-N-[(1S,2R)-2-methoxy-4-[(2S)-2-[(1R,2R)-1-Methoxy-2-methyl-3-oxo-3-[[(1S)-2-phenyl-1-(2-thiazolyl)ethyl]amino]propyl]-1-pyrrolidinyl]-1-[(1S)-1-methylpropyl]-4-oxobutyl]-N-methyl-(9CI)-L-valine amide), mc-MMAF (maleimidocaproyl-mono Tyrauristatin F or N-[6-(2,5-dihydro-2,5-dioxo-1H-pyrrol-1-yl)-1-oxohexyl]-N-methyl-L-valyl-L-valyl-(3R,4S, 5S)-3-Methoxy-5-methyl-4-(methylamino) heptanoyl-(αR, βR, 2S)-β-methoxy-α-methyl-2-pyrrolidinepropanoyl-L-phenylalanine) and mc-Val-Cit-PABA-MMAE (6-maleimidocaproyl-ValcCit-(p-aminobenzyloxycarbonyl)-monomethylauristatin E or N-[[[[[4-[[N-[6-(2,5-Dihydro-2,5-dioxo-1H-pyrrol-1-yl)-1-oxohexyl]-L-valyl-N5-(aminocarbonyl)-L-ornithi Tyl]amino]phenyl]methoxy]carbonyl]-N-methyl-L-valyl-N-[(1S,2R)-4-[(2S)-2-[(1R,2R)-3-[[(1R,2S)-2-hydroxy-1-methyl-2-phenylethyl]amino]-1-methoxy-2-methyl-3-oxopropyl]-1-pyrrolidinyl]-2-methoxy-1-[(1S)-1-methylpropyl]-4-oxobutyl]-N-methyl-L-valinamide). DM1 is a derivative of maytansine, a tubulin inhibitor, while MMAD, MMAE, and MMAF are auristatin derivatives. Preferred payloads of the present invention are selected from the group consisting of mc-MMAF and mc-Val-Cit-PABA-MMAE (see JP5925875B2).

In some embodiments, the present invention comprises an antibody as described herein or antigen-binding fragment and drug moiety antibody drug conjugate, wherein the drug moiety be selected from V-ATP enzyme inhibitor, promote apoptosis agent, Bcl2 inhibitor, MCL1 suppression Preparation, HSP90 inhibitor, IAP inhibitor, mTOR inhibitors, microtubule stabilizer, microtubule destabilizer, the auspicious department's statin of Australia (auristatin), dolastatin (dolastatin), class maytansine (maytansinoid), MetAP (methionine amino Peptase), the inhibitor of protein C RM1 core output, DPPIV inhibitor, proteasome inhibitor, phosphinylidyne group-transfer in mitochondria reaction suppressor, protein synthesis inhibitor, kinase inhibitor, CDK2 inhibitor, CDK9 inhibitor, driving protein inhibitor, Hdac inhibitor, DNA damage agent, DNA alkylating agent, DNA intercalator, DNA minor groove binders and DHFR inhibitor. In one embodiment, the drug moiety of antibody drug conjugate of the present invention is class maytansine (maytansinoid). In another embodiment, the drug moiety of antibody drug conjugate of the present invention is N (2′)-deacetylate-N(2′)-(3-sulfydryl-L-oxopropyl)-maitansine (DM) or N are (2′)-remove acetyl-N2-(4-sulfydryl-4-methyl-1-oxopentyl)-maitansine (DM) (see CN105188763B).

Based on the modes of actions, payloads of cellular toxins used in clinical trials fall into three categories: anti-mitotic (tubulin filaments damaging), DNA damaging or transcription inhibitors. The toxins targeting tubulin filaments include maytansinoids, auristatins, taxol derivatives, etc. In the group of maytansinoids, DM1 and DM4 are the most widely used in the antibody-maytansinoid conjugates that are in clinical trials. Auristatins and auristatin derivatives have strong antivascular effects and among which monomethyl auristatin-E (MMAE) and monomethyl auristatin-F (MMAF) are noteworthy. Taxoids have also been used in various conjugates with different tumor-targeting modules including polyunsaturated fatty acids (PUFAs) and monoclonal antibodies, etc. Calicheamicin, duocarmycin, CC-1065 analogs and duocarmycins are DNA-damaging agents and some of them have a long history of utilization in cancer chemotherapy. The mechanisms of these compounds include modifying DNA bases, intercalating between bases, or forming crosslinks in DNA. Amatoxins are a class of transcription-inhibiting agents. They bind to RNA polymerase II, leading to cell apoptosis. Besides toxins, nanocarriers can also be conjugated to antibodies. Nanocarriers have the capacity to deliver therapeutic agents to the disease site. Therapeutic nanocarriers include lipid-based particles, micelles, nanoparticles, dendrimers, and polymersomes, etc. Moreover, there are some ADCs in clinical trials using protein toxins, enzymes or radionuclides as payloads. Protein toxins function in the similar MOA (mechanism of action) with cellular toxins, they could inhibit protein synthesis and induce cell death. Enzyme-based ADCs alter the microenvironment of disease tissues to disturb their functions. (https://www.creativebiolabs.net/payloads-for-adcs.htm). In addition to toxins, other molecules including steroids may be used as a payload.

One embodiment of the present disclosure provides a pharmaceutical composition for the treatment of a hyperproliferative disorder in a mammal, which comprises an effective amount of an antibody or antigen-binding portion thereof or an antibody-drug conjugate of the present disclosure and a pharmaceutically acceptable carrier. The pharmaceutical composition can be used for the treatment of cancer, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoacanthoma, seminoma, thyroid follicular cancer and teratocarcinoma, and other cancers yet to be determined in which SAS1B is expressed. The instant present disclosure provides pharmaceutical compositions comprising: an effective amount of an antibody, antibody fragment or antibody-drug conjugate of the present disclosure, and a pharmaceutically acceptable carrier, which may be inert or physiologically active.

Effector or therapeutic molecules can be linked to an antibody of the present disclosure using any number of means known to those of skill in the art, for example by covalent or noncovalent attachment. Therapeutic agents or effector molecules that are polypeptides will typically contain a variety of functional groups; such as for example carboxylic acid (COOH), free amine (—NH2) or sulfhydryl (—SH) groups, which are available for reaction with a suitable functional group on an antibody to result in the binding of the effector molecule or therapeutic agent. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule or therapeutic agent. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some embodiments, the present invention includes a linker or connector that binds the toxin to the antibody, antibody-fragment, or peptide. This linker may be selected from a cleavable connector, non-cleavable connector, hydrophilic linker, connector electrically charged in advance and the connector based on dicarboxylic acids. In some embodiments, the connector is derived from a cross-linking reagent selected from the following: N-succinimido-3-(2-pyridyl group two is thio) Propionic ester (SPDP), N-succinimido 4-(2-pyridyl group two is thio) valerate (SPP), N-succinimido 4-(2-Pyridyl group two is thio) butyrate (SPDB), N-succinimido-4-(2-pyridyl group two is thio) 2-sulfo group-butyrate (sulphur Base-SPDB), N-succinimidyl iodoacetate (SIA), N-succinimido (4-iodoacetyl) Aminobenzoate (SIAB), maleimide PEG NHS, N-succinimido 4-(maleimidomethyl)cyclohexane carboxylate (SMCC), N-sulfosuccinimide base 4-(maleimidomethyl)cyclohexane carboxylate (sulfo group-SMCC) or 17-(2,5-dioxo-2,5-dihydro-1H-pyrroles-1-base) four oxo-4,7,10,13- of -5,8,11,14-, four azepine, 17 carbon-1-acid 2,5-dioxo Pyrrolidin-1-yl ester (CX1-1) (see CN105188763B). Site-specific conjugation may be performed, including but not limited to, AJICAP (https://onlinelibrary.wiley.com/doi/abs/10.1002/anie.201814215).

Linker unit (LU) describes the direct or indirect linkage of an antibody to a drug. Attachment of the linker to the mAb can be accomplished in a variety of ways, for example through cysteine residues that are liberated by surface lysine, reductive coupling to oxidized carbohydrates, and reduction of interchain disulfide linkages. Various ADC linkage systems are known in the art, including hydrazone, disulfide, and peptide-based linkages (see JP5925875B2).

Particularly preferred linker molecules include, for example, N-succinimidyl 3-(2-pyridyldithio)propionate (SPDP) (see, e.g., Carlsson et al., Biochem. J., 173, 723-737 (1978)), N-succinimidyl 4-(2-pyridyldithio)butanoate (SPDB) (see, e.g., U.S. Pat. No. 4,563,304), N-succinimidyl 4-(2-pyridyldithio)pentanoate (SPP) (see, e.g., CAS Registry number 341498-08-6), N-succinimidyl 4-(N-maleimidomethyl)cyclohexane-1-carboxylate (SMCC) (see, e.g., Yoshitake et al., Eur. J. Biochem., 101, 395-399 (1979)), and N-succinimidyl 4-methyl-4-[2-(5-nitro-pyridyl)-dithio]pentanoate (SMNP) (see, e.g., U.S. Pat. No. 4,563,304) The most preferred linker molecules for use in the inventive composition are SPP, SMCC, and SPDB (see U.S. Pat. No. 7,494,649B2).

In some circumstances, it is desirable to free the effector molecule or therapeutic agent from the antibody when the antibody-drug conjugate has reached its target site. Therefore, antibody-drug conjugates can comprise linkages that are cleavable in the vicinity of the target site. Cleavage of the linker to release the effector molecule or therapeutic molecule from the antibody can be accomplished by, for example, enzymatic activity or conditions to which the antibody-drug conjugate is subjected either inside the target site or in the vicinity of the target site.

In view of the large number of methods that have been reported for attaching a variety of radiodiagnostic compounds, radiotherapeutic compounds, labels (such as enzymes or fluorescent molecules) drugs, toxins, and other agents to antibodies one skilled in the art will be able to determine a suitable method for attaching a given therapeutic agent or effector molecule to an antibody or other polypeptide.

Antibodies of the present disclosure can be labeled with a detectable moiety. Detectable moieties include, for example, fluorescent compounds, including fluorescein, fluorescein isothiocyanate, rhodamine, 5-dimethylamine-1-naphthalenesulfonyl chloride, phycoerythrin, lanthanide phosphors, bioluminescent markers (e.g., luciferase, green fluorescent protein (GFP), yellow fluorescent protein (YFP)), enzymes (e.g., horseradish peroxidase, β-galactosidase, luciferase, alkaline phosphatase, glucose oxidase), a magnetic agent (e.g. gadolinium), lanthanides (e.g., europium and dysprosium), manganese, paramagnetic particles (e.g., superparamagnetic iron oxide), polypeptide epitopes recognized by a secondary reporter (such as leucine zipper pair sequences, radiolabeled amino acids, binding sites for secondary antibodies, metal binding domains, epitope tags). Detectable moieties can be attached to antibodies by spacer arms of various lengths to reduce potential steric hindrance.

Methods of Treatment

Antibodies and antigen-binding fragments thereof can be conjugated to a cytotoxic agent, such as duocarmycin, maytansinoids, and auristatins, to form a drug having specific cytotoxicity towards SAS1B-expressing cells by targeting the drug to SAS1B. Cytotoxic conjugates comprising such antibodies and/or antigen-binding fragments thereof and a drug or cytotoxin can be used as a therapeutic for treatment of hyperproliferative disorders such as any SAS1B-positive cancer, including but not limited to, uterine cancer, pancreatic cancer, ovarian cancer. Cytotoxic conjugates can also be used as contraceptive agents by inhibiting or killing SAS1B-expressing oocytes.

One embodiment of the present disclosure provides methods of treating or preventing hyperproliferative disorders comprising administering an effective amount of an antibody or antigen-binding fragment thereof of the present disclosure or an antibody-drug conjugate of the present disclosure to a mammal in need thereof.

An “effective amount” is an amount sufficient to effect beneficial or desired results. For example, an effective amount is one that achieves the desired therapeutic effect. An effective amount can be administered in one or more administrations, applications or dosages. An effective amount of a pharmaceutical composition (i.e., an effective dosage) depends on the pharmaceutical composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with an effective amount of the pharmaceutical compositions described herein can include a single treatment or a series of treatments.

“Treatment” means the administration of one or more pharmaceutical compositions to a subject. The term treatment also includes an adjustment (e.g., increase or decrease) in the dose or frequency of one or more pharmaceutical agents that a subject can be taking, the administration of one or more new pharmaceutical agents to the subject, or the removal of one or more pharmaceutical agents from the subject's treatment plan. Treatment also refers to any amelioration of one or more symptoms of a hyperproliferative disease, improvement in patient survival, or the reversal of the disease. Treatment also refers to contraceptive effects, which can occur by killing or inhibiting oocytes and/or through modulation of SAS1B function.

A subject can be an animal, for example, a mammal, a human, monkey, dog, cat, horse, cow, pig, goat, rabbit, or mouse.

A “pharmaceutical composition” is a sterile or aseptic composition of an antibody or antigen-binding fragment thereof or antibody-drug conjugate of the present disclosure formulated with a pharmaceutically acceptable carrier, which can be safely administered to a subject. The pharmaceutical composition does not cause undesirable side effects when administered to a patient that outweigh the beneficial effects.

“Pharmaceutically-acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as combination thereof. Isotonic agents, such as sugars, polyalcohols, or sodium chloride can be present in compositions of the present disclosure. Examples of suitable carriers include, for example: Dulbecco's phosphate buffered saline, pH about 7.4, containing or not containing about 1 mg/ml to 25 mg/ml human serum albumin; 0.9% saline (0.9% w/v sodium chloride (NaCl)), and 5% (w/v) dextrose. Carriers can also contain an antioxidant such as tryptamine and a stabilizing agent such as TWEEN20® (polysorbate).

Administration can be by any method, including, for example, parenteral administration such as intravenous, intramuscular, intraperitoneal, subcutaneous, intra-articular, intrasynovial, intratumoral, peritumoral, intralesional, or perilesional. Compositions of the present disclosure can be administered intravenously as a bolus or by continuous infusion over a period of time.

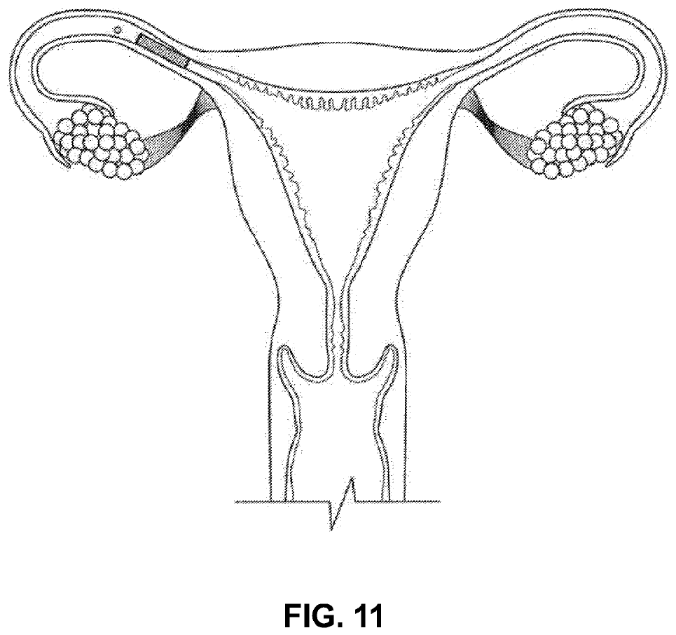

Administration can also be local, such as administration by injection into one or both fallopian tubes, or intra-uterine or intra-vaginal administration, particularly for contraceptive applications. In one embodiment, the pharmaceutically-acceptable carrier is a biocompatible implant, bulking mechanism, foam, hydrogel, polymer, or other occlusive substance that contains the antibody, antigen-binding fragment or antibody-drug conjugate of the present disclosure. The implant, bulking mechanism, foam, hydrogel, polymer, or other substance is administered into the fallopian tube where it occludes the fallopian tube and releases the antibody, antigen-binding fragment or antibody-drug conjugate in the lumen of the fallopian tube. In one embodiment, the antibody, antigen-binding fragment or antibody-drug conjugate is contained in micropores of the implant, bulking mechanism, foam, hydrogel, polymer, or other substance, from which it is released into the lumen of the fallopian tube. In another embodiment, the antibody, antigen-binding fragment or antibody-drug conjugate is non-covalently bound within the implant, bulking mechanism, foam, hydrogel, polymer, or other substance and released into the lumen of the fallopian tube. The biocompatible implant, bulking mechanism, foam, hydrogel, polymer, or other occlusive substance can be a composition as described in U.S. patent application Publication Nos. US20190038454A1, US20180185096A1, US20190053790A1, US20180028715A1, PCT Application Nos. WO2017083753A1 and WO2019070632A1, and U.S. patent No. U.S. Ser. No. 10/155,063B2, which are incorporated by reference in their entireties. The biocompatible implant, bulking mechanism, foam, hydrogel, polymer, or other occlusive substance can be administered into a lumen of the fallopian tube by way of a needle or cannula. An example of such a device implanted in a fallopian tube is shown in FIG. 11.

Sterile compositions for parenteral administration can be prepared by incorporating the antibody, antigen-binding fragment or antibody-drug conjugate of the present disclosure in the required amount in the appropriate solvent, followed by sterilization by microfiltration. As solvent or vehicle include, for example, water, saline, phosphate buffered saline, dextrose, glycerol, ethanol, and the like, as well as a combination thereof. Isotonic agents, such as sugars, polyalcohols, or sodium chloride can be present in compositions of the present disclosure. These compositions may also contain adjuvants, in particular wetting, isotonizing, emulsifying, dispersing and stabilizing agents. Sterile compositions for parenteral administration can also be prepared in the form of sterile solid compositions, which can be dissolved at the time of use in sterile water or any other injectable sterile medium.

The antibodies, antigen-binding fragments thereof or antibody-drug conjugates of the present disclosure can also be orally administered as a solid composition (tablets, pills, powders gelatin capsules, sachets) or granules) or liquid compositions (pharmaceutically acceptable solutions, suspensions, emulsions, syrups and elixirs containing inert diluents such as water, ethanol, glycerol, vegetable oils or paraffin oil). These compositions may comprise substances other than diluents, for example wetting, sweetening, thickening, flavoring or stabilizing products.

The doses depend on the desired effect, the duration of the treatment and the route of administration used; they are generally between 5 mg and 1000 mg per day orally for an adult with unit doses ranging from 1 mg to 250 mg of active substance.

Antibodies, antibody-binding portions thereof, or antibody-drug conjugates of the present disclosure can be used for the treatment of a hyperproliferative disorder in a mammal. The antibodies, antibody-binding portions thereof, or antibody-drug conjugates of the present disclosure can also be used to treat the neovascularization of a tumor. The antibodies, antibody-binding portions thereof, or antibody-drug conjugates of the present disclosure can also be used for oocyte-specific contraception.

Similarly, the present disclosure provides a method for inhibiting the growth of selected cell populations comprising contacting target cells, or tissue containing target cells, with an effective amount of an antibody, antigen-binding fragment or antibody-drug conjugate of the present disclosure, or an antibody, antigen-binding fragment or a therapeutic agent comprising a cytotoxic conjugate, either alone or in combination with other cytotoxic or therapeutic agents.

Methods for inhibiting the growth of selected cell populations expressing SAS1B can be practiced in vitro, in vivo, or ex vivo. “Inhibiting growth” means slowing the growth of a cell, decreasing cell viability, causing the death of a cell, lysing a cell and inducing cell death, over a short period of time (e.g., minutes to hours) or a long period of time (e.g., days to months).

Examples of in vitro uses include treatments of autologous bone marrow prior to their transplant into the same patient in order to kill diseased or malignant cells; treatments of bone marrow prior to its transplantation in order to kill competent T cells and prevent graft-versus-host-disease (GVHD); treatments of cell cultures in order to kill all cells except for desired variants that do not express the target antigen; or to kill variants that express undesired antigen.

Examples of clinical ex vivo use include the removal of tumor cells or lymphoid cells from bone marrow prior to autologous transplantation in cancer treatment or in treatment of autoimmune disease, or to remove T cells and other lymphoid cells from autologous or allogeneic bone marrow or tissue prior to transplant in order to prevent graft versus host disease (GVHD).

For clinical in vivo use, the antibody, the antigen-binding fragment, or the antibody-drug conjugate of the present disclosure can be supplied as solutions that are sterile and contain appropriate levels of endotoxin. Examples of suitable protocols of antibody-drug conjugate administration are as follows. Antibodies, antigen-binding fragments thereof or antibody-drug conjugates can be given weekly for 4 weeks as an i.v. bolus each week. Bolus doses are given in 50 to 100 ml of normal saline to which 5 to 10 ml of human serum albumin can be added. Dosages can be 10 μg to 100 mg per administration, i.v. (range of 100 ng to 1 mg/kg per day). Dosages can range from 50 μg to 30 mg. Dosages can range from 1 mg to 20 mg. After four weeks of treatment, the patient can continue to receive treatment on a weekly basis. Specific clinical protocols with regard to route of administration, excipients, diluents, dosages, times, etc., can be determined by one of ordinary skill in the art as the clinical situation warrants.

The antibodies or antigen-binding fragments of the present disclosure can also be used to detect SAS1B in a biological sample in vitro or in vivo. Antibodies or fragments thereof of the present disclosure can be used to determine the level of SAS1B in a tissue or in cells derived from the tissue. The tissue can be diseased tissue, a tumor or a biopsy of a tumor. The tissue or biopsy thereof can be frozen, fixed, permeabilized or non-permeabilized.

The above-described method can be used to diagnose a cancer in a subject known to or suspected to have a cancer, wherein the level of SAS1B measured in said patient's tissues, blood or serum is compared with that of a normal reference subject or standard. The method can then be used to determine whether a tumor or cells of tissue of the patient expresses SAS1B, which may suggest that the tumor or patient will respond well to treatment with the antibodies, antigen-binding fragments or antibody-drug conjugates of the present disclosure.

A method for diagnosis is also provided in which labeled antibodies, antigen-binding fragments thereof, or antibody-drug conjugates are administered to a subject suspected of having a cancer, and the distribution of the label within the body of the subject is measured or monitored. A method is also provided in which labeled antibodies, antigen-binding fragments thereof, or antibody-drug conjugates are administered to a subject for determining in the subject, fertility or lack thereof.

Vaccines

Embodiments of the invention encompass vaccines useful for contraception. In one aspect, a propeptide of SAS1B such as a polypeptide comprising or consisting of amino acids 34-53 and/or 64-86 of human SAS1B is/are delivered to a subject to elicit an active immune response, such as an antibody response to block SAS1B function on oocytes or a T-cell mediated attack on the oocytes.

SAS1B antigens can be produced in large amounts and purified for use in vaccine preparations. SAS1B antigens also have utility in immunoassays, e.g. to detect or measure in a sample of body fluid from a vaccinated subject the presence of antibodies to the antigen, and thus to diagnose and/or to monitor immune responses of the subject subsequent to vaccination.

The preparation of vaccines containing an immunogenic polypeptide as the active ingredient is known to one skilled in the art (see, for example, Vaccine Design, Michael F. Powell and Mark J. Newman Eds., Plenum Press, New York, 1995, pp 821-902).

The immunopotency of SAS1B antigens such as a polypeptide comprising or consisting of amino acids 34-53 and/or 64-86 of human SAS1B can be determined by monitoring the immune response in test animals following immunization with the SAS1B antigen or by use of any immunoassay known in the art. Generation of a humoral (antibody) response and/or cell-mediated immunity, may be taken as an indication of an immune response. Test animals may include mice, hamsters, dogs, cats, monkeys, rabbits, chimpanzees, etc., and eventually human subjects.

Methods of introducing the vaccine may include oral, intravaginal, intradermal, intramuscular, intraperitoneal, intravenous, subcutaneous, intranasal, and via scarification (scratching through the top layers of skin, e.g., using a bifurcated needle) or any other standard routes of immunization. The immune response of the test subjects can be analyzed by various approaches such as: the reactivity of the resultant immune serum to the egg protein antigen, as assayed by known techniques, e.g., immunosorbent assay (ELISA), immunoblots, radioimmunoprecipitations, etc., or in the case where the SAS1B antigen displays antigenicity or immunogenicity, by protection of the immunized host against fertilization in the immunized host.

As one example of suitable animal testing of a vaccine, the vaccine may be tested in rabbits for the ability to induce an antibody response to SAS1B antigen. Male specific-pathogen-free (SPF) young adult New Zealand White rabbits may be used. The test group each receives a fixed concentration of the vaccine. A control group receives an injection of 1 mM Tris-HCl pH 9.0 without the SAS1B antigen.

Blood samples may be drawn from the rabbits every one or two weeks, and serum analyzed for antibodies to the SAS1B antigen. The presence of antibodies specific for the antigen may be assayed, e.g., using an ELISA.

Suitable preparations of such vaccines include injectables, either as liquid solutions or suspensions; solid forms suitable for solution in, suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

Examples of adjuvants which may be effective, include, but are not limited to: aluminum hydroxide, N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(−2′-dipalmitoyl-sn-glycero-3-hydroxyphosphoryloxy)-ethylamine.

The effectiveness of an adjuvant may be determined by measuring the induction of antibodies directed against an immunogenic polypeptide containing a SAS1B epitope such as a polypeptide comprising or consisting of amino acids 34-53 and/or 64-86 of human SAS1B, the antibodies resulting from administration of this polypeptide in vaccines which are also comprised of the various adjuvants.

The polypeptides may be formulated into the vaccine as neutral or salt forms. Pharmaceutically acceptable salts include the acid addition salts (formed with free amino groups of the peptide) and which are formed with inorganic acids, such as, for example, hydrochloric or phosphoric acids, or organic acids such as acetic, oxalic, tartaric, maleic, and the like. Salts formed with free carboxyl groups may also be derived from inorganic bases, such as, for example, sodium potassium, ammonium, calcium, or ferric hydroxides, and such organic bases as isopropylamine, trimethylamine, 2-ethylamino ethanol, histidine, procaine and the like.

The vaccines may be multivalent or univalent. Multivalent vaccines are made from recombinant viruses that direct the expression of more than one antigen. The patient to which the vaccine is administered is preferably a mammal, most preferably a human, but can also be a non-human animal including but not limited to cows, horses, sheep, pigs, fowl (e.g., chickens), goats, cats, dogs, hamsters, mice and rats. The vaccine formulations comprise an effective immunizing amount of SAS1B antigen and a pharmaceutically acceptable carrier or excipient. Vaccine preparations comprise an effective immunizing amount of one or more antigens and a pharmaceutically acceptable carrier or excipient. Pharmaceutically acceptable carriers are well known in the art and include but are not limited to saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

In a specific embodiment, a SAS1B antigen is provided in a first container; a second container comprises diluent comprising an aqueous solution of 50% glycerin, 0.25% phenol, and an antiseptic (e.g., 0.005% brilliant green).

The precise dose of vaccine preparation to be employed in the formulation will also depend on the route of administration, and the nature of the patient, and should be decided according to the judgment of the practitioner and each patient's circumstances according to standard clinical techniques. An effective immunizing amount is that amount sufficient to produce an immune response to the antigen in the host to which the vaccine preparation is administered.

Use of purified antigens as vaccine preparations can be carried out by standard methods. For example, the purified protein(s) should be adjusted to an appropriate concentration, formulated with any suitable vaccine adjuvant and packaged for use. Suitable adjuvants may include, but are not limited to: mineral gels, e.g., aluminum hydroxide; surface active substances such as lysolecithin, pluronic polyols; polyanions; peptides; oil emulsions; alum, and MDP. The immunogen may also be incorporated into liposomes, or conjugated to polysaccharides and/or other polymers for use in a vaccine formulation. In instances where the recombinant antigen is a hapten, i.e., a molecule that is antigenic in that it can react selectively with cognate antibodies, but not immunogenic in that it cannot elicit an immune response, the hapten may be covalently bound to a carrier or immunogenic molecule; for instance, a large protein such as serum albumin will confer immunogenicity to the hapten coupled to it. The hapten-carrier may be formulated for use as a vaccine.

Effective doses (immunizing amounts) of the vaccines may also be extrapolated from dose-response curves derived from animal model test systems.

The invention also provides a pharmaceutical pack or kit comprising one or more containers comprising one or more of the ingredients of the vaccine formulations of the invention. Associated with such container(s) can be a notice in the form prescribed by a governmental agency regulating the manufacture, use or sale of pharmaceuticals or biological products, which notice reflects approval by the agency of manufacture, use or sale for human administration.

The present invention thus provides a method of immunizing an animal, comprising administering to the animal an effective immunizing dose of a vaccine of the present invention.

The following are provided for exemplification purposes only and are not intended to limit the scope of the present disclosure described in broad terms above.

### EXAMPLES

### Example 1

Two synthetic peptides derived from the primary sequence of human SAS1B were used for immunizations. These peptides consisted of amino acid residues 34-53 (GTSFPDGLTPEGTQASGDK) and 64-86 (LEETPESSFLIEGDIIRPSPFRL), respectively, which are both found in the propeptide region of SAS1B. The two peptides are non-overlapping and are predicted to exhibit favorable solubility. The polypeptides were synthesized in vitro with appended C-terminal CG or CGG residues as spacers resulting in the immunogens: CGGLEETPESSFLIEGDIIRPSPFRL and CGGTSFPDGLTPEGTQASGDK. These peptides were conjugated to keyhole limpet hemocyanin (KLH) as a carrier.

To generate antibody-secreting B cells, 3 female BALB/c mice aged 8-12 weeks were immunized with a 1:1 mixture of the two peptide-KLH conjugates emulsified in complete Freund's adjuvant (CFA). A subsequent booster immunization with a 1:1 mixture of peptide-KLH conjugates emulsified in incomplete Freund's adjuvant was given three weeks later.

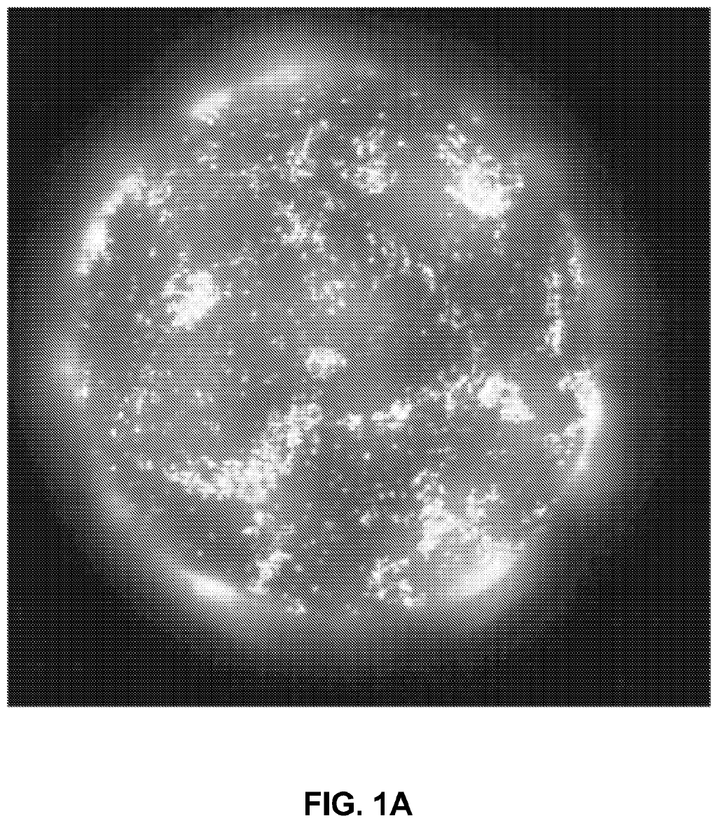

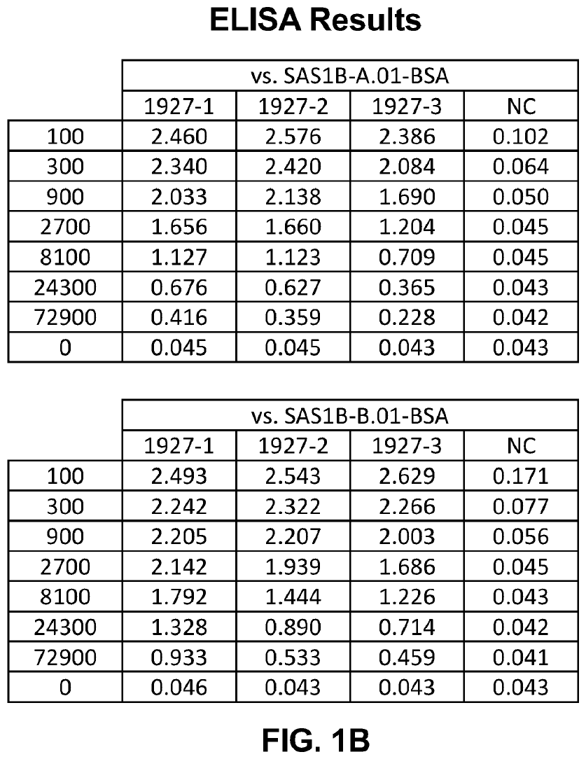

Following immunization, on day 40, peptide-specific serum titer was assessed by ELISA. 3/3 mice exhibited strong titers (>1:8100) against both peptides. A single mouse was identified as the best responder and was given a 25 mg booster of 1:1 peptide-KLH conjugates in phosphate-buffered saline intravenously. Five days following the intravenous boost, lymphocytes from the spleen and lymph nodes from the single mouse were fused with NS1 an immortalized myeloma cell line to generate hybridomas. The hybridomas were cultured under selective conditions (HAT) for 10 to 11 days. Hybridomas were then screened for secretion of antibody that binds to either of the two SAS1B peptides by ELISA (FIG. 1B). 94 positive hybridomas were identified and these were cultured to generate antibody-containing supernatant for further testing. Antibody binding was confirmed by ELISA following 5 days culture.

### Example 2

To enable efficient screening and testing of antibodies directed at SAS1B, over-expressing stable cell pools were generated using HEK293T cells. First, endogenous expression of SAS1B by HEK293 was assessed by QPCR. Results of this experiment put the range of endogenous expression at the limit of detection for the assay, thus parental HEK293T do not express appreciable amounts of endogenous SAS1B. To generate stable pools, expression plasmid vectors encoding full-length human SAS1B (NP_001002036.3) and splice variant IV (hereafter designated SAS1BX1; XP_011509507.1) with C-terminal c-myc (EQKLISEEDL) and FLAG (DYKDDDDK) peptide tags were introduced to parental HEK293T cells via liposomal transfection. Under selective pressure via antibiotic, stable cell line pools were developed. These cell pools exhibited both intracellular and cell-surface staining for c-myc peptide by flow-cytometry.

Binding of anti-peptide antibodies in hybridoma supernatants to cell-expressed SAS1B was assessed by intracellular flow-cytometry using SAS1B-overexpressing HEK293T (SAS1B-HEK293T) cells as positive binding targets; parental HEK293T served as the negative control. The positive threshold was defined as >10% positive events (cells) on HEK293T-SAS1B and >3 fold increase in the percentage of positive events of SAS1B-HEK293T versus parental HEK293T cells. In total, 15 positive supernatants, of 94 screened, were identified.

The 15 positive supernatants were then subjected to further flow-cytometry analysis using three natural SAS1B-expressing cell lines (AsPc-1, Colo205, SNU539) as well as SAS1B-HEK293T and SAS1B X1-isoform-overexpressing (SAS1BX1-HEK293T) cell lines to identify cell surface SAS1B-binding antibodies. Five supernatants exhibited binding to cell surface SAS1B, and minimal to no binding to control cell lines. As benchmarks, previously disclosed anti-SAS1B antibodies to SB2 and 6B1, were included. Staining was observed for these antibodies on both overexpressing cell lines and parental HEK293T indicating a degree of non-specificity of these antibodies.

Hybridomas were then subcloned twice by limiting dilution in selective media to generate the final hybridoma clones: OV115, OV119, OV121, OV123, OV124.

### Example 3

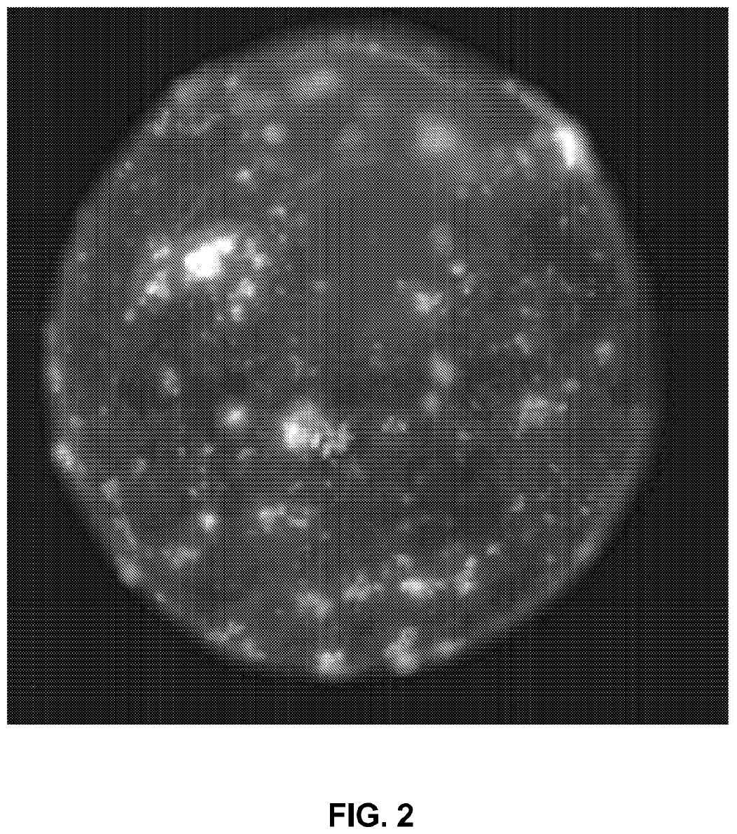

Supernatants from these final hybridoma clones were then used to stain human oocytes (FIGS. 2, 3A and 3B, and 4-6). To remove oocytes from the zona pellucida, laser ablation and manual pressure using microtools were employed to extract the oocyte leaving the oolemma intact. Zona-free oocytes were then pre-equilibrated in 100 μl drops under oil of KSOM+5% serum for 1 hr at 37° C./7% CO₂. Anti-SAS1B antibodies were then added as either pre-equilibrated supernatant or primary Ab at 5 μg/ml in KSOM+2.5% serum and incubated for 2 hr at 37° C./7% CO₂. Following extensive washing, oocytes were transferred to 4% paraformaldehyde for 15 minutes at room temperature. After repeated washing secondary antibodies to mouse IgG (AF488; 1:200) in PBS/2.5% serum were added and incubated for 1 hr at room temperature or overnight at 4° C. in the dark. A similar procedure was used to counterstain the oocytes for the JUNO cell surface marker. Oocytes were then mounted on slides for imaging using IDIBI mount media:PBS/serum at 1:1 ratio. Imaging was done using a fluorescent scope with appropriate excitation and filters. FIGS. 2, 3A and 3B, and 4-6 are fluorescent microscopy images showing Juno visualized by the red staining and SAS1B visualized by the green staining.

Furthermore, 3 eggs were live stained with the murine anti-Ovalbumin (IgG) at 5 μg/ml followed by Juno (post-fixation) as a negative control. All 3 oocytes were negative for the Ovalbumin and strongly positive for JUNO and DAPI.

FIG. 2 shows Zona-intact M2 egg stained with OV119 (green) and Juno (red).

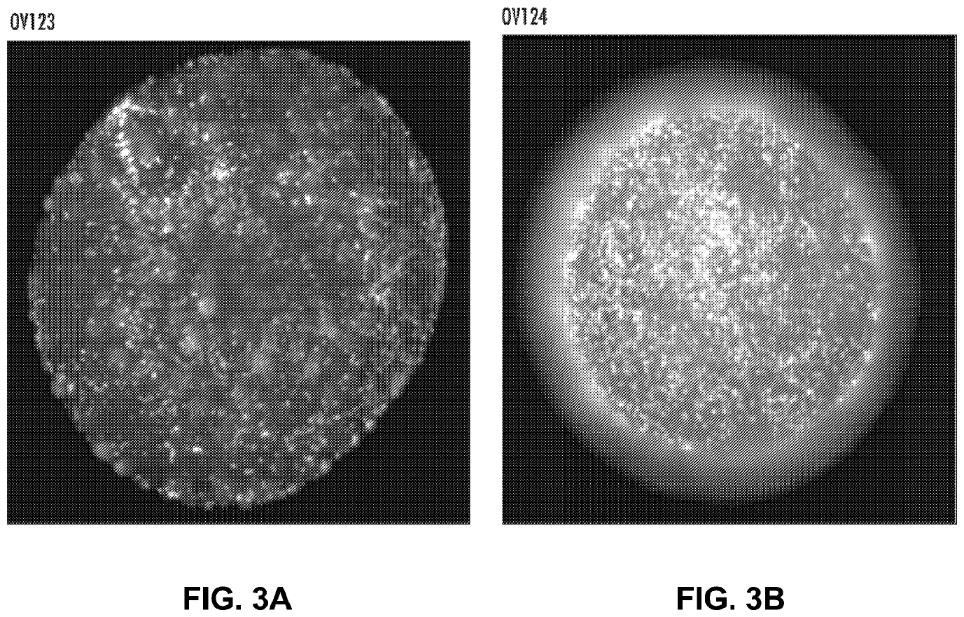

FIGS. 3A and 3B show Zone-Free M2 eggs stained with OV123 and OV124 (green).

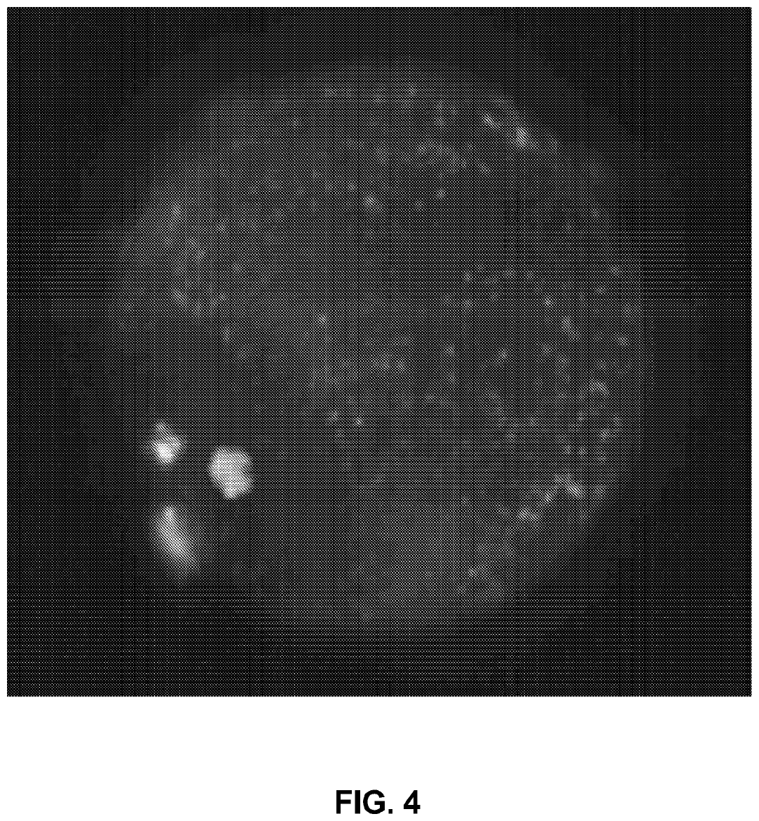

FIG. 4 shows Zone-Free M2 egg stained with OV115 (green) and Juno (red).

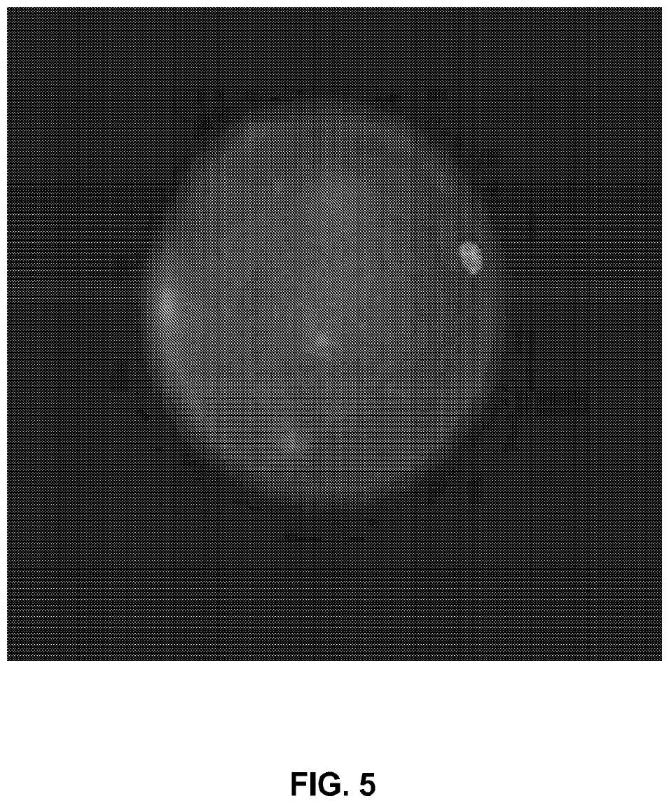

FIG. 5 shows Ovalbumin IgG—Negative control (green) and Juno (red).

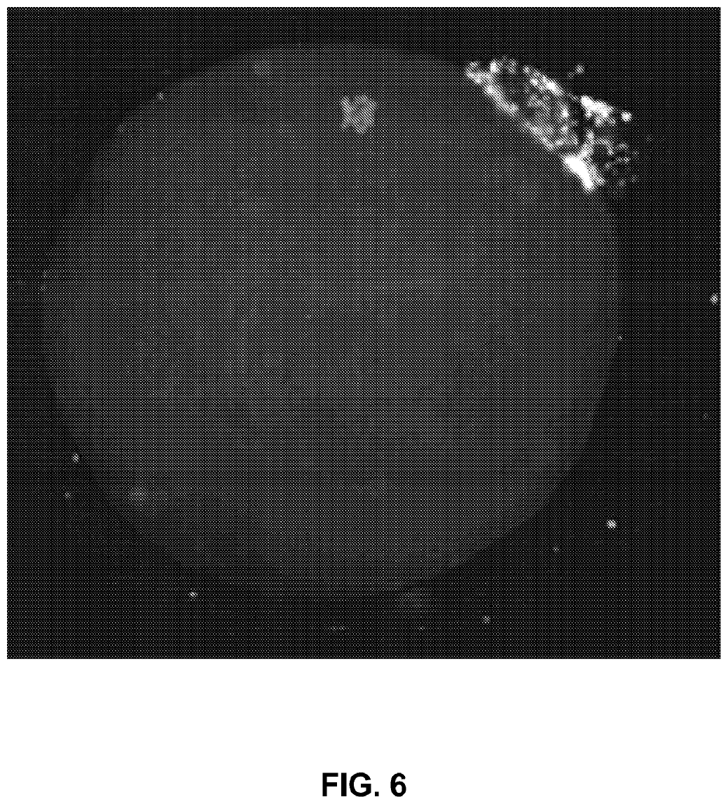

FIG. 6 shows Zona-intact M2 egg stained with OV121 (green) and Juno (red) demonstrating positive staining of SAS1B in the polar body.

### Example 4

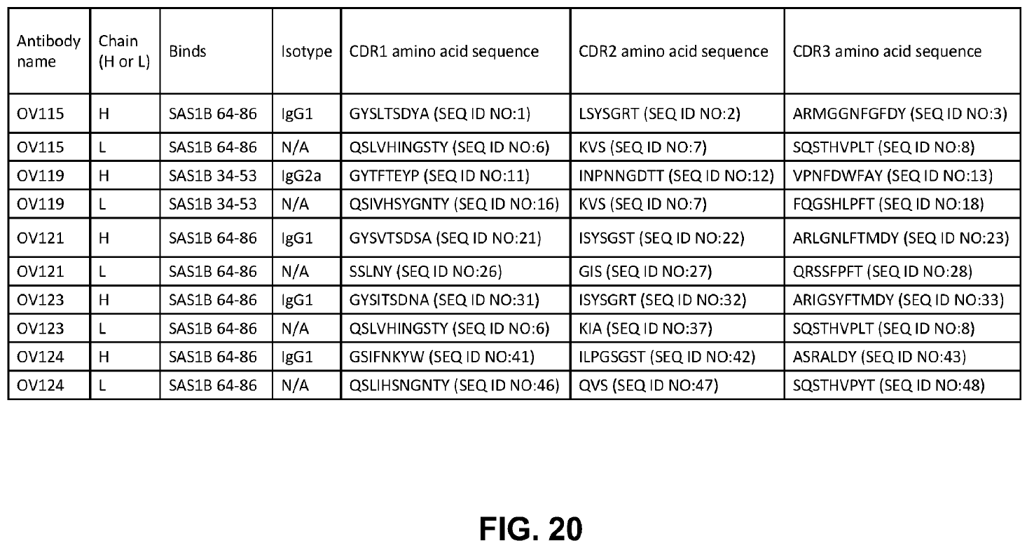

The DNA encoding antibody variable-heavy and -light portions were sequenced for monoclonal antibodies OV115, OV119, OV121, OV123, and OV124. The primary amino acid sequence corresponding to the DNA sequence was determined, as well as the individual CDRs 1, 2, and 3 for variable-heavy and variable-light portions. Polynucleotide and polypeptide sequence information is provided in the attached sequence listing, as described in the Brief Description of the Polypeptide and Polynucleotide Sequences. The table in FIG. 20 contains the sequences of the CDRs for each antibody.

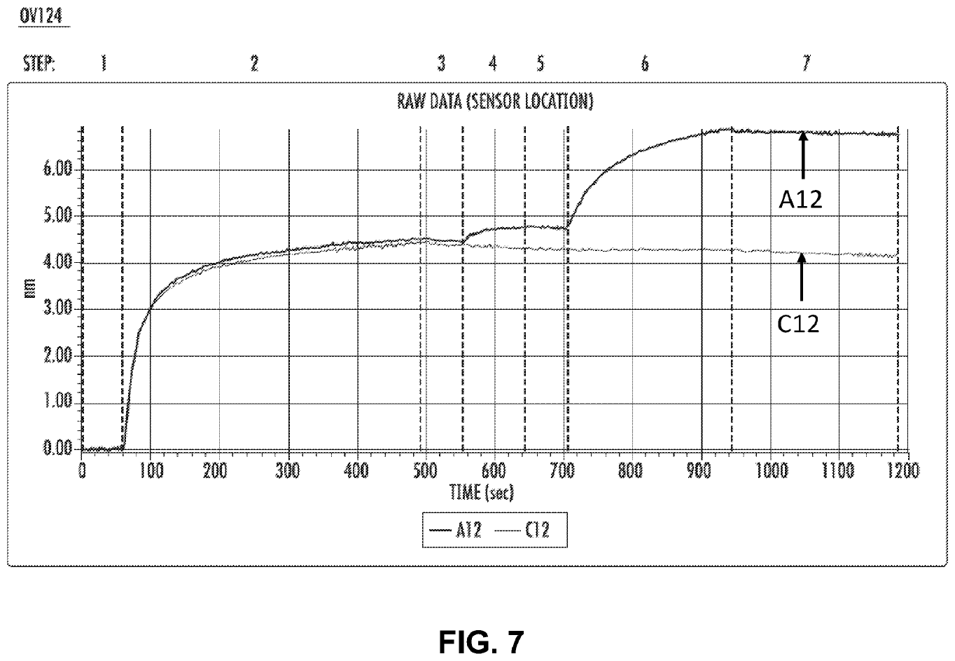

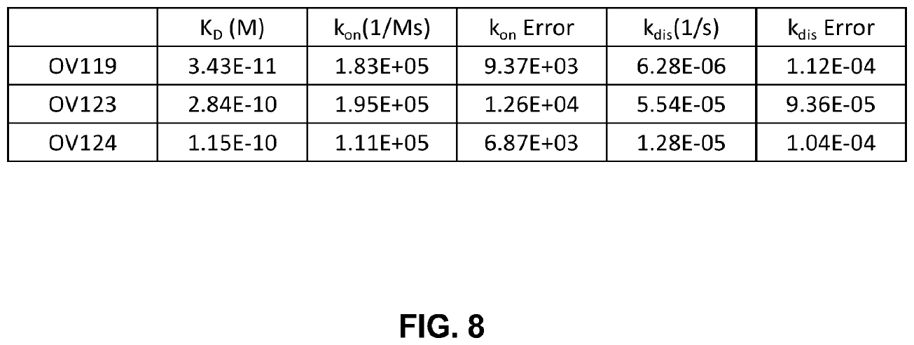

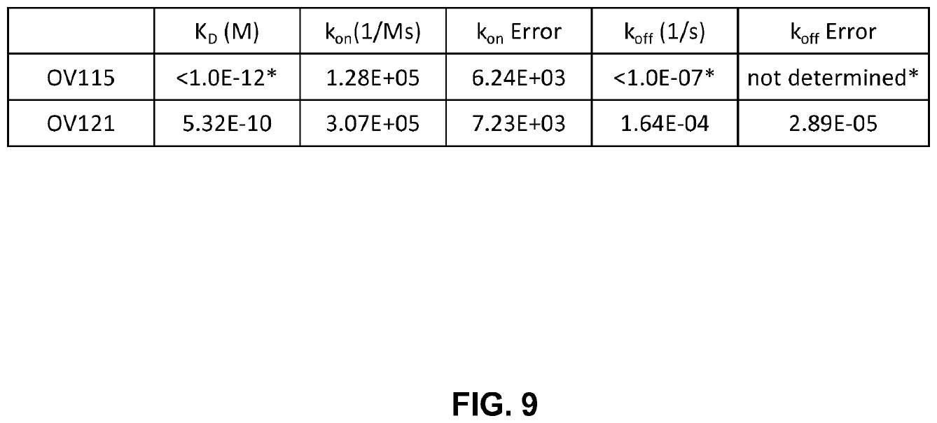

Protein A-Purified antibodies were subjected to binding kinetic analysis via biolayer interferometry (Fortebio's Octet instrument) and the parameters of binding to peptide-BSA conjugate were determined (FIGS. 8 and 9). Antibodies OV119, 123, and 124 bound their respective antigens. Association constants (Kon) were in the range of 1-2 e5. The dissociation constants (Kdis) from these three clones were too slow to be accurately fit (<1×10⁻⁶ sec⁻¹). With the caveat that Kdis were inaccurate due to these long residency times, KDs were calculated (FIGS. 8 and 9). A sensorgram of OV124 is shown in FIG. 7.

### Example 5

One of the monoclonal antibodies was conjugated to a proprietary, water-soluble payload. The resulting DAR (drug antibody ratio) was 0.9. The toxin conjugation to heavy and light chain was confirmed by RPC/MS. In analytical SEC-HPLC, no aggregation was observed.

### Example 6

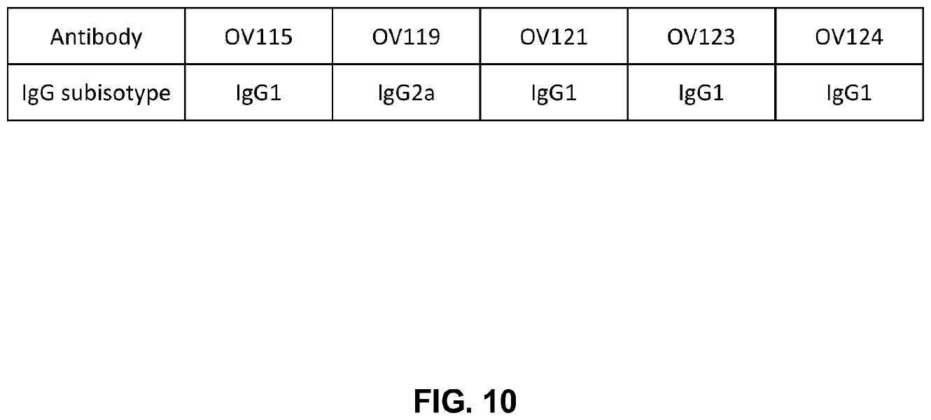

To determine antibody IgG subisotypes, a commercially-available bead-based assay using the Luminex assay was employed. FIG. 10 is a table showing antibody IgG subisotypes.

### Example 7

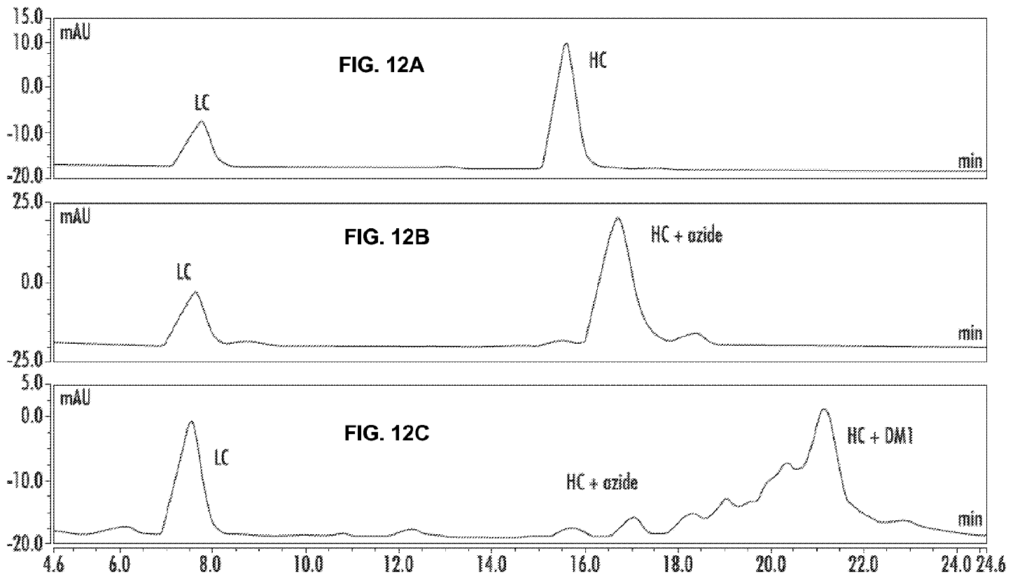

Reverse-phase LC of OV119 conjugated to DM1 using AJICAP site-specific conjugation. Mouse anti-SAS1B antibodies (OV115, OV119) and control antibodies of irrelevant specificity (GMA640) were covalently conjugated to the cytotoxic payload DM1 using a proprietary site-specific process involving use of azide intermediates. The conjugated antibodies and intermediates were analyzed using reverse phase liquid chromatography (LC). Plots show relative absorption units (mAU) of light at 280 nm versus time. Plots show traces from naked antibody (FIG. 12A), azide intermediate (FIG. 12B), and DM1-conjugated antibody (FIG. 12C) with OV119 as an example. Where applicable, the various species are annotated.

### Example 8

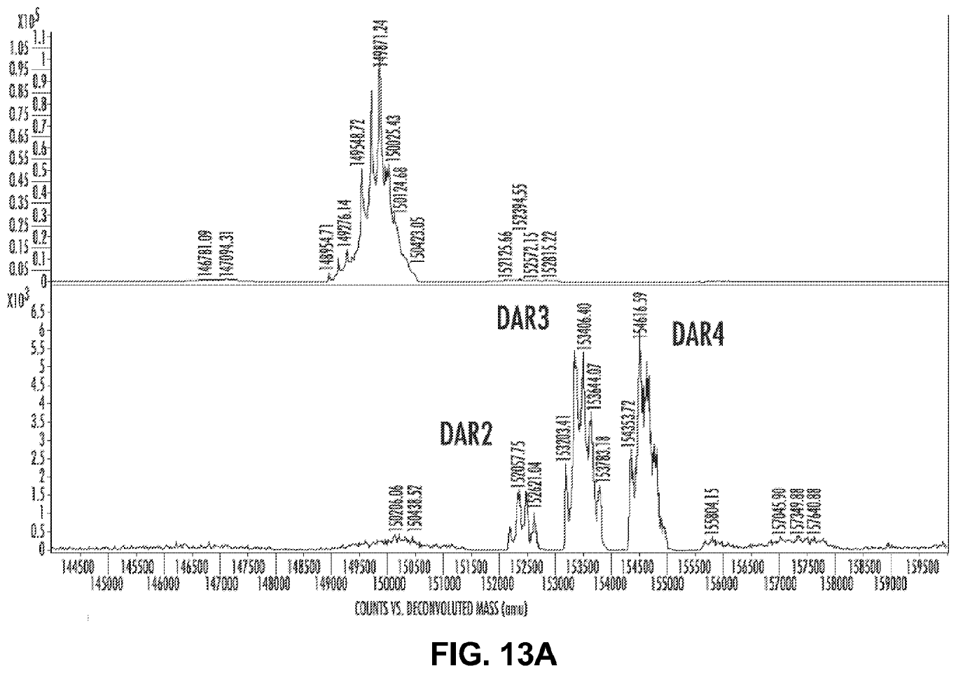

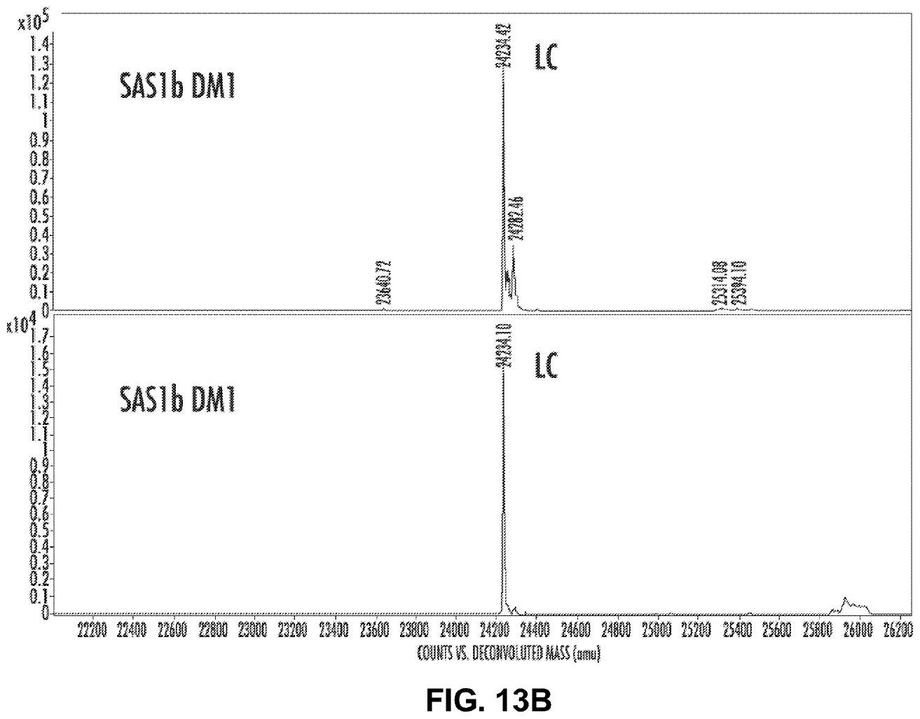

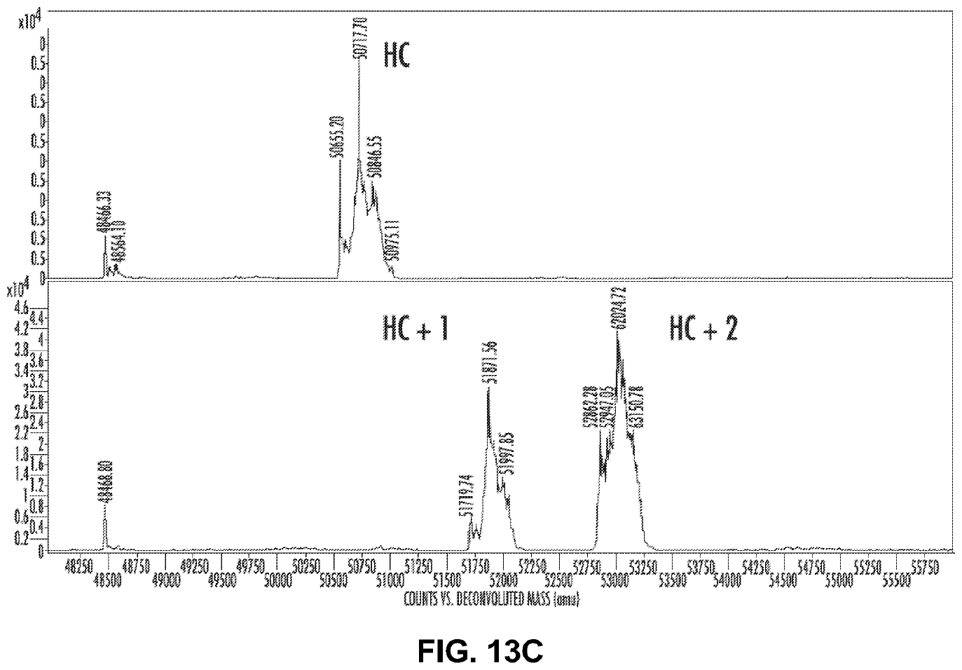

Quantitative time of flight (qTOF) traces showing OV119 conjugated to DM1. Mouse anti-SAS1B antibodies (OV115, OV119) and control antibodies of irrelevant specificity (GMA640) were covalently conjugated to the cytotoxic payload DM1 using a proprietary site-specific process involving use of azide intermediates. Conjugated OV119-DM1 was analyzed in both native (FIG. 13A) and under reducing conditions (FIGS. 13B and 13C). For reference naked OV119 was analyzed (top trace, FIG. 13A). In FIG. 13A, drug to antibody ratios (DAR) are annotated based on observed masses for the conjugated species in the bottom trace. Light chains (FIG. 13B) and heavy chains (FIG. 13C) were analyzed under reducing conditions to demonstrate that heavy chain conjugation was incomplete with either 1 or 2 DM1 molecules per heavy chain which in various combinations lead to the DARs of 2, 3, and 4 in FIG. 13A.

### Example 9

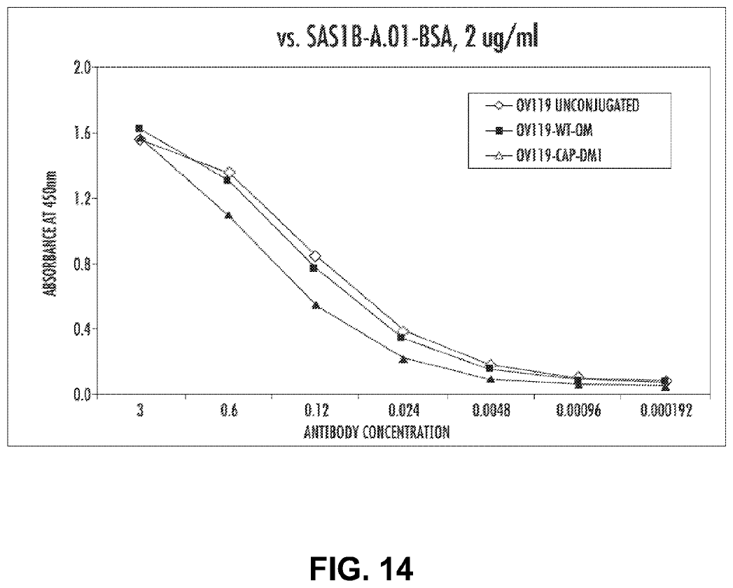

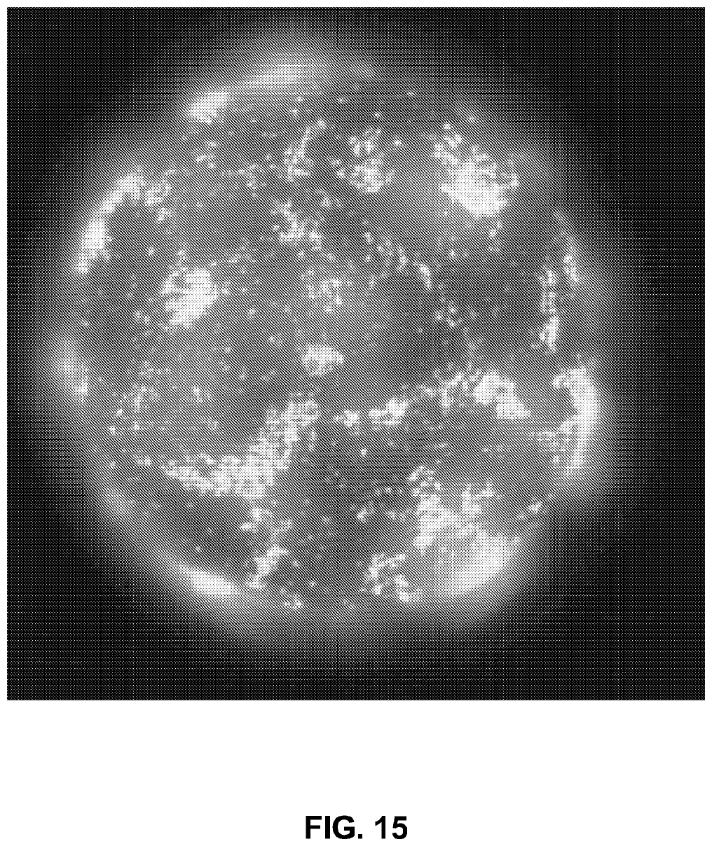

Conjugation of OV119 to DM1 does not significantly affect its binding to antigen. Colorimetric ELISA data comparing binding of naked OV119 to OV119 conjugated using stochastic chemistry (OV119-WT-OM) and AJICAP site-specific conjugation (OV119-CAP-DM1). The solid phase was coated with BSA conjugated to SAS1B peptide A (CGGLEETPESSFLIEGDIIRPSPFRL). Normalized concentrations of the three antibody moieties were titrated by serial 5-fold dilution. A secondary antibody specific for mouse IgG and conjugated to horse radish peroxidase was then applied. After development using TMB substrate and reaction stopping with H₂SO₄, absorbance of light at 450 nm was measured and plotted against antibody concentration (FIG. 14).

### Example 10

Surface staining of SAS1B on live human oocytes (OV119). To determine whether antibodies to the membrane proximal portion of SAS1B bind specifically to human oocytes (known to express SAS1B), live oocytes derived from patients undergoing IVF were stained and imaged. The zona pellucida was manually removed via laser and oocytes were blocked in KSOM with 5% donkey serum. Primary anti-SAS1B antibodies (in this case OV119) were then applied either as neat hybridoma supernatants or at 5 ug/ml in KSOM+2.5% serum for 1 h at 37° C. Following extensive washing, oocytes were fixed with 4% paraformaldehyde. Secondary donkey anti-mouse IgG labeled with alexafluor488 was then applied for one hour and incubated for 1 hour at 4° C. After washing oocytes were imaged using fluorescence microscopy (FIGS. 15, 1A).

### Example 11

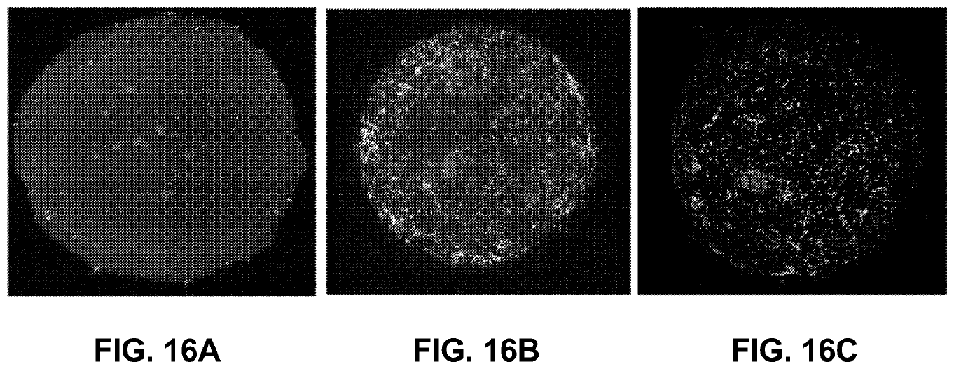

Compressed confocal image stacks of OV119, OV119-DM1 and EEA1 on permeabilized human oocytes. To test the hypothesis that the SAS1B-specific monoclonal antibody OV119, and the corresponding ADC prototype OV119-DM1 are internalized by human oocytes following cell surface binding, we assessed co-localization between the antibody/ADC and EEA1, a marker of early endosomes. First the zona pellucida was removed from human immature oocytes derived from IVF patients consenting to research. Oocytes were then blocked (KSOM+5% donkey serum) and live stained with OV119 or SAS1B-DM1 antibody (or unstained=Non-treated control) for 1 HR under standard culture conditions (37° C., 5% CO₂). All oocytes were then washed, fixed (4% paraformaldehyde), permeabilized (0.05% TritonX100) and blocked (5% donkey serum) before incubation in donkey anti-mouse alexafluor 488-conjugated secondary antibody. Oocytes were then incubated in sheep anti EEA-1 and then donkey anti-sheep alexa594-conjugated secondary antibody overnight. Chromatin was stained with DAPI. Oocytes were then 3D imaged using a Leica SP8X laser confocal microscope. Z-stacked confocal images were then overlayed (compressed) to give an overall image of the stained oocytes. FIGS. 16A-C are compressed confocal microscopy image stacks of OV119, OV119-DM1 and EEA1 on permeabilized human oocytes where FIG. 16A is a control, FIG. 16B is OV119 (green stain), and FIG. 16C is OV119-DM1 (green stain). Red stain represents EEA1 while blue stain represents DAPI in each image.

### Example 12

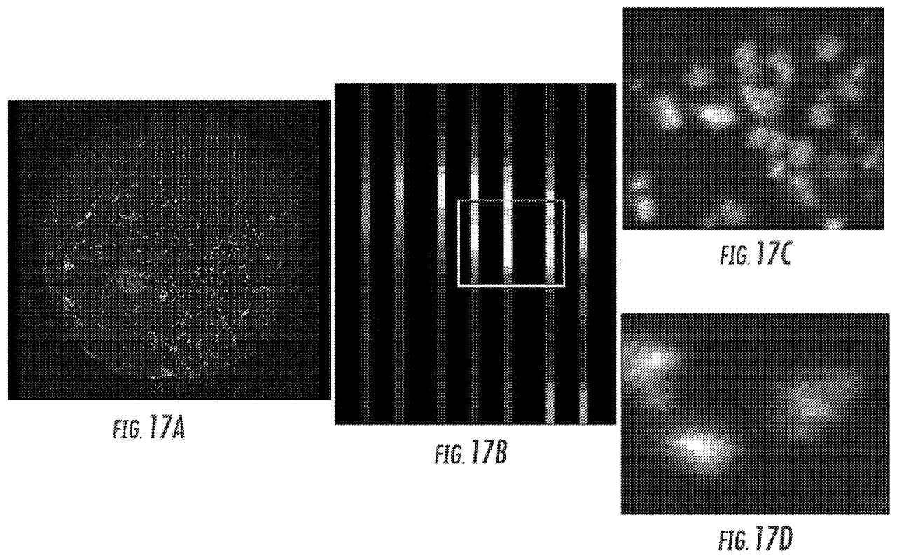

Evidence for internalization of OV119-DM1, colocalization with EEA1 (entry to early endosomes. To test they hypothesis that the SAS1B-specific monoclonal antibody OV119, and the corresponding ADC prototype OV119-DM1 are internalized by human oocytes following cell surface binding, we assessed co-localization between the antibody/ADC and EEA1, a marker of early endosomes. First the zona pellucida was removed from human immature oocytes derived from IVF patients consenting to research. Oocytes were then blocked (KSOM+5% donkey serum) and live stained with OV119 or SAS1B-DM1 antibody (or unstained=Non-treated control) for 1 HR under standard culture conditions (37° C., 5% CO₂). All oocytes were then washed, fixed (4% paraformaldehyde), permeabilized (0.05% TritonX100) and blocked (5% donkey serum) before incubation in donkey anti-mouse alexafluor 488-conjugated secondary antibody. Oocytes were then incubated in sheep anti EEA-1 and then donkey anti-sheep alexa594-conjugated secondary antibody overnight. Chromatin was stained with DAPI. Oocytes were then 3D imaged using a Leica SP8X laser confocal microscope. Co-localization between OV119 or OV119-DM1 and EEA1 is evidence for entry of internalized antibody/ADC to the endosomal pathway. FIG. 17A shows a compressed confocal image of a OV119-DM1-stained oocyte. FIG. 17B shows Z-plane cross-sections of the same oocyte zoomed to show co-localization (appears yellow). Similarly, FIGS. 17C and 17D are zoomed single z-plane confocal images showing co-localization between OV119-DM1 and EEA1.

### Example 13

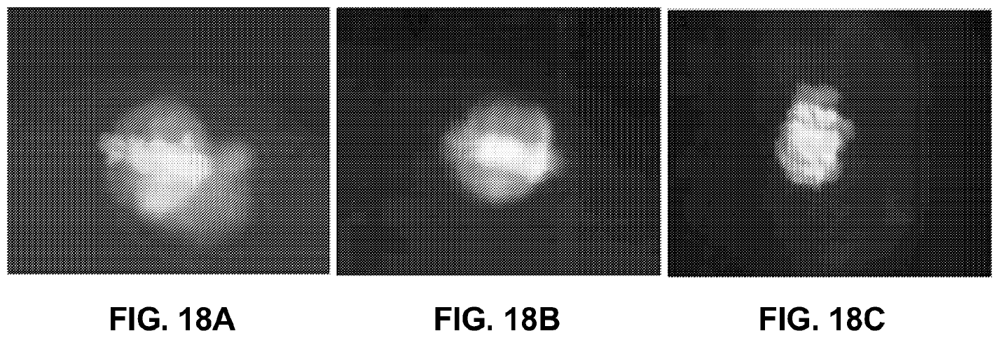

Evidence for disruption of the meiotic spindle (MH) following exposure of human oocytes to OV119-DM1. To test they hypothesis that the SAS1B-DM1 disrupts meiotic spindle formation following internalization by human oocytes, we assessed MII spindle formation in antibody/ADC treated oocytes. First the zona pellucida was removed from human immature oocytes derived from IVF patients consenting to research. Oocytes were then blocked (KSOM+5% donkey serum) and live stained with OV119 or SAS1B-DM1 antibody (or unstained=Non-treated control) overnight under standard culture conditions (37° C., 5% CO₂). All oocytes were then washed, fixed (4% paraformaldehyde), permeabilized (0.05% TritonX100) and blocked (5% donkey serum) before incubation in donkey anti-mouse tubulin alexafluor 488-conjugated primary antibody. Chromatin was stained with DAPI. Oocytes were then imaged at 60× by fluorescence microscopy. FIG. 18A shows non-treated control, where both spindles appear normal and barrel shaped with good chromosome alignment. FIG. 18B shows GMA640-DM1 (ADC of irrelevant specificity), also a normal barrel shaped spindle with aligned chromosomes. FIG. 18C shows OV119-DM1, with abnormal spindles with misaligned chromosomes (i.e., chromosomes are not centrally lined-up in the spindle and appear chaotic or not incorporated in spindle).

### Example 14

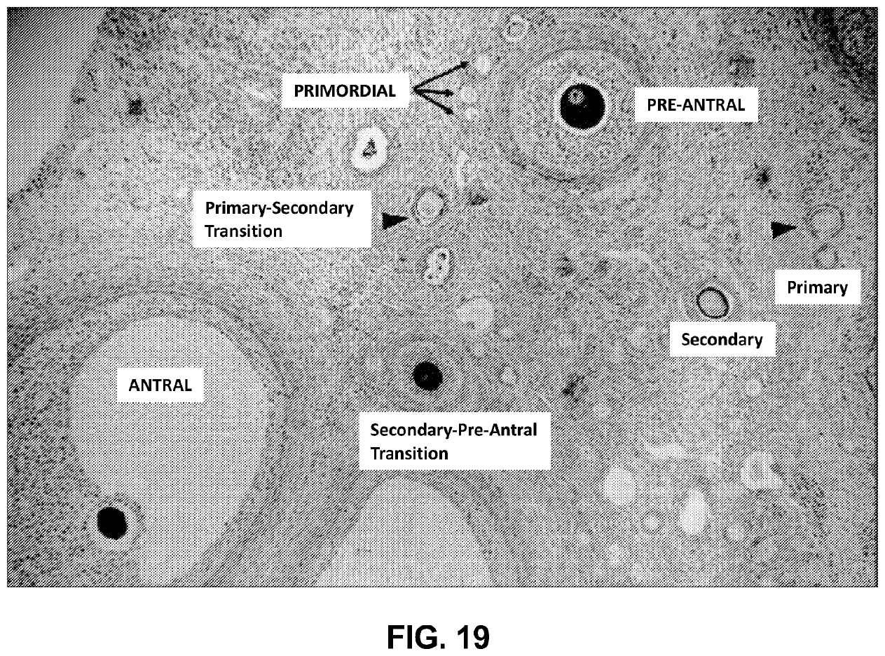

OV119 binds oocytes in a stage-dependent manner (Macaca nemistrina). Immunohistochemistry was performed to determine the stage of development at which anti-SAS1B antibodies bind to NHP oocytes. Formalin-fixed paraffin-embedded sections of Macaca nemestrina ovarian cortex was labeled with OV119, OV115 and 6B1. An HRP-conjugated secondary anti-mouse antibody was then applied. The tissue was then developed with DAB producing a brown insoluble dye. The sections were then counterstained with hematoxylin to highlight cell nuclei. A representative, annotated image stained with OV119 is shown in FIG. 19. Numerous follicles/oocytes at various stages of development are apparent. For each of the antibodies tested: Primordial follicles/oogonia were negative, primary follicles/oocytes were negative, light staining was observed in oocytes undergoing the transition from the primary to secondary follicle stage where both the oocyte and follicle diameter grow, a theca layer forms, and a 2nd layer of granulosa cells is laid down. Staining in the secondary oocyte was more intense and during the transition from secondary through to antral follicle oocyte staining becomes very strong. There was no evidence of background or non-specific staining in either the nontreated control (NTC) or negative control antibody (GMA640).

### Example 15

Antibody and ADC serum titers in mice following intraperitoneal injection. On day 0 Balb/c mice (n=6/group) were injected with 3 mg/kg of either OV115, OV115-DM1, GMA640, or GMA640-DM1 intraperitoneally. On alternating timepoints (days 1, 3, 6, 10, 14, 21, and 28) 3 mice/group were test bled. Serum was prepared and applied in an antigen-specific ELISA assay with either BSA-SAS1B-Peptide B or Ovalbumin serving as the antigen on the solid phase. Serum titers were assessed at 50% of the max OD and averaged by group. Data were then plotted longitudinally as a way to estimate serum half-life of the respective antibodies and ADC. In general ADC serum half-life was observed to be substantially similar to the corresponding naked antibody.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure, including patents, published patent applications, and publications are each individually incorporated by reference herein in their entireties and as such are intended to provide an efficient way of supplementing the enabling disclosure of this invention as well as provide background detailing the level of ordinary skill in the art.

1. An isolated antibody or antigen-binding portion thereof that specifically binds human SAS1B.

2. (canceled)

3. The isolated antibody or antigen-binding portion thereof of claim 1, wherein said antibody or antigen-binding portion thereof specifically binds to a polypeptide comprising or consisting of amino acids 34-53 and/or 64-86 of human SAS1B.

4. The isolated antibody or antigen-binding portion thereof of claim 3, wherein the antibody or antigen-binding portion thereof is a monoclonal antibody, a chimeric antibody, a humanized antibody, a synthetic antibody, a single chain antibody, a diabody, or a CDR-grafted antibody.

5. (canceled)

6. The isolated antibody or antigen-binding portion thereof of claim 1 comprising OV115, OV119, OV121, OV123, and/or OV124, or an isolated antibody or antigen-binding portion with an amino acid sequence with at least 95% identity to the amino acid sequence of OV115, OV119, OV121, OV123, and/or OV124.

7-11. (canceled)

12. The isolated antibody or antigen-binding portion thereof of claim 6, wherein the antibody or antigen-binding portion thereof is a monoclonal antibody, a chimeric antibody, a humanized antibody, a synthetic antibody, a single chain antibody, a diabody, or a CDR-grafted antibody.

13-35. (canceled)

36. A method for inhibiting proliferation or killing a SAS1B positive cancer cell, said method comprising contacting said cancer cell with an effective amount of an antibody or antigen-binding portion thereof or antibody-drug conjugate of any preceding claim, wherein said antibody or antigen-binding fragment thereof or antibody-drug conjugate binds with SAS1B, thereby inhibiting proliferation of the cancer cell or killing the cancer cell.

37. (canceled)

38. The method of claim 36, wherein said cancer is carcinoma, sarcoma, uterine cancer, ovarian cancer, lung cancer, adenocarcinoma, adenocarcinoma of the lung, squamous carcinoma, squamous carcinoma of the lung, malignant mixed mullerian tumor, leukemia, lymphoma, or endometrioid carcinoma.

39-64. (canceled)

65. The isolated antibody or antigen-binding portion of claim 1 comprising one or more variable light chain CDRs having an amino acid sequence at least 70% identical to, SEQ ID NO:6, SEQ ID NO:7, SEQ ID NO:8, SEQ ID NO:16, SEQ ID NO:18, SEQ ID NO:26, SEQ ID NO:27, SEQ ID NO:28, SEQ ID NO:37, SEQ ID NO:46, SEQ ID NO:47, and/or SEQ ID NO:48.

66. The isolated antibody or antigen-binding portion of claim 65, wherein the isolated antibody or antigen binding portion specifically binds human SAS1B, and/or a polypeptide comprising or consisting of amino acids 34-53 and/or 64-86 of human SAS1B, with an affinity (Kd) of at least about 10-6 M, or at least about 10-7 M, or at least about 10-8 M, or at least about 10-9 M, or at least about 10-10 M, or at least about 10-11 M, or at least about 10-12 M.

67-71. (canceled)

72. An isolated antibody or antigen-binding portion thereof comprising:
a chimeric polypeptide comprising:
a human antibody amino acid sequence or sequences comprising one or more constant regions; and
one or more non-human, such as mouse, antibody amino acid sequences selected from:
(a) variable heavy chain CDRs represented by SEQ ID NO:1, SEQ ID NO:2, SEQ ID NO:3, SEQ ID NO:11, SEQ ID NO:12, SEQ ID NO:13, SEQ ID NO:21, SEQ ID NO:22, SEQ ID NO:23, SEQ ID NO:31, SEQ ID NO:32, SEQ ID NO:33, SEQ ID NO:41, SEQ ID NO:42, and SEQ ID NO:43; and
(b) variable light chain CDRs represented by SEQ ID NO:6, SEQ ID NO:7, SEQ ID NO:8, SEQ ID NO:16, SEQ ID NO:18, SEQ ID NO:26, SEQ ID NO:27, SEQ ID NO:28, SEQ ID NO:37, SEQ ID NO:46, SEQ ID NO:47, and SEQ ID NO:48;
wherein one or more of the selected non-human antibody amino acid sequences listed in (a) or (b) are between 60% and 100% identical to those set forth; and
wherein the antibody or antigen-binding portion thereof specifically binds human SAS1B and/or a polypeptide comprising or consisting of amino acids 34-53 and/or 64-86 of human SAS1B.

73. (canceled)

74. The antibody or antigen-binding portion thereof of claim 72, wherein the antibody or antigen-binding portion thereof specifically binds human SAS1B and/or a polypeptide comprising or consisting of amino acids 34-53 and/or 64-86 of human SAS1B with an affinity (Kd) of at least about 10⁻¹² M.

75. A therapeutic agent comprising the antibody or antigen-binding portion thereof of claim 72 conjugated to a molecule or structure that is cytotoxic to oocytes and/or cancer cells.

76. The therapeutic agent of claim 75, wherein the molecule or structure is an antibody, a protein, a pro-drug, a drug, a toxin, a protein toxin, a liposome, a radioactive isotope, or an enzyme.

77. The therapeutic agent of claim 75, wherein the molecule or structure is an antibody-conjugatable tubulin inhibitor.

78. The therapeutic agent of claim 77, wherein the tubulin inhibitor is a maytansinoid, such as DM1 or DM4.

79. A composition comprising the therapeutic agent of claim 75 and a pharmaceutically acceptable carrier.

80. (canceled)

81. A method for inhibiting proliferation or killing a SAS1B positive cancer cell, the method comprising contacting the cancer cell with an effective amount of an antibody or antigen-binding portion thereof of claim 72, or therapeutic agent comprising the antibody or antigen-binding portion, wherein said antibody or antigen-binding fragment thereof or therapeutic agent binds with SAS1B, thereby inhibiting proliferation of the cancer cell or killing the cancer cell.

82. (canceled)

83. The method of claim 81, wherein the cancer is carcinoma, sarcoma, uterine cancer, ovarian cancer, lung cancer, adenocarcinoma, adenocarcinoma of the lung, squamous carcinoma, squamous carcinoma of the lung, malignant mixed mullerian tumor, leukemia, lymphoma, or endometrioid carcinoma.

84-101. (canceled)

102. The isolated antibody or antigen-binding portion thereof of claim 1 having a serum half-life on the order of weeks.

103-105. (canceled)


SEQUENCE LISTING (50 sequences)

SEQ ID NO: 1 - protein, 9 aa, Mus musculus
GYSLTSDYA

SEQ ID NO: 2 - protein, 7 aa, Mus musculus
LSYSGRT

SEQ ID NO: 3 - protein, 11 aa, Mus musculus
ARMGGNFGFDY

SEQ ID NO: 4 - DNA, 355 bp, Mus musculus
gatgtgcagcttcaggagtcgggacctggcctggtgaaaccttctcagtctctgtccctcacctgcactgtcacaggctactcactcaccagtgattatgcctggaactggatccggcagtttccaggaaaaaaagtggagtggatgggctacctaagctacagtggtagaactagctacaacccatctctcaaaagtcgaatctctatcactcgagacacatccaggaaccagttcttcctgcagttgaattctgtgactactgaggacacagccacatattactgtgcaagaatggggggtaacttcgggtttgactactggggccaaggcaccactctcactgtctctacag

SEQ ID NO: 5 - protein, 118 aa, Mus musculus
DVQLQESGPGLVKPSQSLSLTCTVTGYSLTSDYAWNWIRQFPGKKVEWMGYLSYSGRTSYNPSLKSRISITRDTSRNQFFLQLNSVTTEDTATYYCARMGGNFGFDYWGQGTTLTVST

SEQ ID NO: 6 - protein, 11 aa, Mus musculus
QSLVHINGSTY

SEQ ID NO: 7 - protein, 3 aa, Mus musculus
KVS

SEQ ID NO: 8 - protein, 9 aa, Mus musculus
SQSTHVPLT

SEQ ID NO: 9 - DNA, 337 bp, Mus musculus
ggcattgtgatgacccaggctccactctccctgcctgtcagtcttggagatcaagcctccatctcttgcagatctagtcagagccttgtacacattaatggaagcacctatttacattggtacctgcagaagccaggccagtctccaaacctcctgatctacaaagtttcctaccgattttctggggtcccagacaggttcagtggcagtggatcagggacagatttcacactcaagatcaccagagtggaggctgaggatctgggagtttatttctgctctcaaagtacacatgttcctctcacgttcggctcggggacaaagttggaaataaaac

SEQ ID NO: 10 - protein, 112 aa, Mus musculus
GIVMTQAPLSLPVSLGDQASISCRSSQSLVHINGSTYLHWYLQKPGQSPNLLIYKVSYRFSGVPDRFSGSGSGTDFTLKITRVEAEDLGVYFCSQSTHVPLTFGSGTKLEIK

SEQ ID NO: 11 - protein, 8 aa, Mus musculus
GYTFTEYP

SEQ ID NO: 12 - protein, 9 aa, Mus musculus
INPNNGDTT

SEQ ID NO: 13 - protein, 9 aa, Mus musculus
VPNFDWFAY

SEQ ID NO: 14 - DNA, 349 bp, Mus musculus
gaggtgcagctgcaggagtctggacctgaactggtgaagcctggggcttcagtgaagatatcctgcaagacttctggatacactttcactgaataccccatgcactgggtgaagcagagccatggaaagagccttgagtggattggaggtattaatcctaacaatggtgatactacttacaaccagaggttcaagggcaaggccacattgactgtagacaagtcctccggcacagcctacatggagctccgcagcctgacatctgaggattctgcagtctattactgtgtacctaatttcgactggtttgcttactggggccaagggactctggtcacagtctctccag

SEQ ID NO: 15 - protein, 116 aa, Mus musculus
EVQLQESGPELVKPGASVKISCKTSGYTFTEYPMHWVKQSHGKSLEWIGGINPNNGDTTYNQRFKGKATLTVDKSSGTAYMELRSLTSEDSAVYYCVPNFDWFAYWGQGTLVTVSP

SEQ ID NO: 16 - protein, 11 aa, Mus musculus
QSIVHSYGNTY

SEQ ID NO: 17 - skipped

SEQ ID NO: 18 - protein, 9 aa, Mus musculus
FQGSHLPFT

SEQ ID NO: 19 - DNA, 337 bp, Mus musculus
gatgttttgatgacccaaagtccactctccctgcctgtcagtcttggagatcaagcctccatctcttgcagatctagtcagagcattgtacatagttatggaaacacctatttagaatggtatctgcagaaaccaggccagtctccaaagctcctgatctacaaagtttccaaccgattttctggggtcccagacaggttcagtggcagtggatcagggacagatttcacactcaggatcagcagagtggaggctgaggatctgggaatttattactgctttcaaggttcacatcttccgttcacgttcggaggggggaccaagctggaaataaaac

SEQ ID NO: 20 - protein, 112 aa, Mus musculus
DVLMTQSPLSLPVSLGDQASISCRSSQSIVHSYGNTYLEWYLQKPGQSPKLLIYKVSNRFSGVPDRFSGSGSGTDFTLRISRVEAEDLGIYYCFQGSHLPFTFGGGTKLEIK

SEQ ID NO: 21 - protein, 9 aa, Mus musculus
GYSVTSDSA

SEQ ID NO: 22 - protein, 7 aa, Mus musculus
ISYSGST

SEQ ID NO: 23 - protein, 11 aa, Mus musculus
ARLGNLFTMDY

SEQ ID NO: 24 - DNA, 355 bp, Mus musculus
gaggtgcagctgcaggagtctggacctggcctggtgaaaccttctcagtctctgtccctcacctgcactgtcactggctattcagtcaccagtgattctgcctggaactggatccggcagtttccaggaaacaaactggagtggatgggctacataagctacagtggtagtactacctacaacccctctctcaaaagtcgaatctctatcactcgagacacatccgagaaccagttcttcctgcagttgcgttctgtgactactgaggacacagccacatattactgtgcaagacttggtaacctctttactatggactactggggtcaaggaacctcagtcacagtctctgcag

SEQ ID NO: 25 - protein, 118 aa, Mus musculus
EVQLQESGPGLVKPSQSLSLTCTVTGYSVTSDSAWNWIRQFPGNKLEWMGYISYSGSTTYNPSLKSRISITRDTSENQFFLQLRSVTTEDTATYYCARLGNLFTMDYWGQGTSVTVSA

SEQ ID NO: 26 - protein, 5 aa, Mus musculus
SSLNY

SEQ ID NO: 27 - protein, 3 aa, Mus musculus
GIS

SEQ ID NO: 28 - protein, 8 aa, Mus musculus
QRSSFPFT

SEQ ID NO: 29 - DNA, 319 bp, Mus musculus
gagattttgctcacccagtccccagtaatcttagctgcatctcctggggagaaggtcaccatcacctgcagtgccagctcaagtctaaattacatgaactggtaccagcagagaccaggatcctcccccaaaatatggatttatggtatatccaacctggcttctggagttcctgctcgcttcagtggcagtgggtctgggacatctttctctttcacaatcaacagcatggaggctgaagatgttgccacttattactgtcagcaaaggagtagtttcccattcacgttcggctcggggacaaagctggaaattaaac

SEQ ID NO: 30 - protein, 106 aa, Mus musculus
EILLTQSPVILAASPGEKVTITCSASSSLNYMNWYQQRPGSSPKIWIYGISNLASGVPARFSGSGSGTSFSFTINSMEAEDVATYYCQQRSSFPFTFGSGTKLEIK

SEQ ID NO: 31 - protein, 9 aa, Mus musculus
GYSITSDNA

SEQ ID NO: 32 - protein, 7 aa, Mus musculus
ISYSGRT

SEQ ID NO: 33 - protein, 11 aa, Mus musculus
ARIGSYFTMDY

SEQ ID NO: 34 - DNA, 355 bp, Mus musculus
gaggtgcagctgcaggagtctggacctggcctggtgaaaccttctcagtctctgtccctcacctgcactgtcactggctactcaatcaccagtgataatgcctggaactggatccggcagtttccaggaaacaaactggagtggatgggctacataagctacagtggtcgcactagctacaacccatctctcaaaagtcgaatctctgtcactcgagacacttccaagaaccggttcttcttgcagttgaattctgtgactactgaggacacagccacatattactgtgcaagaatcgggtcttactttactatggactactggggtcaaggaacctcagtcaccgtctcctcag

SEQ ID NO: 35 - protein, 118 aa, Mus musculus
EVQLQESGPGLVKPSQSLSLTCTVTGYSITSDNAWNWIRQFPGNKLEWMGYISYSGRTSYNPSLKSRISVTRDTSKNRFFLQLNSVTTEDTATYYCARIGSYFTMDYWGQGTSVTVSS

SEQ ID NO: 36 - skipped

SEQ ID NO: 37 - protein, 3 aa, Mus musculus
KIA

SEQ ID NO: 38 - skipped

SEQ ID NO: 39 - DNA, 337 bp, Mus musculus
gatgttgtgatgacccaaactccactctccctgtctgtcggtcttggagatcaagcctccatctcttgcagatctagtcagagccttgtacacattaatggaagcacctatttacattggtacctgcagaagccaggccagtctccaaagctcctgatctacaaaattgccaaccgattttccggggtcccagacaggttcagtggcagtggatcagggacagatttcacactcaagatcagcagagtggaggctgaggatctgggagcttatttctgctctcaaagtacacatgttccgctcacgttcggtgctgggaccaagctggaaatcaaac

SEQ ID NO: 40 - protein, 112 aa, Mus musculus
DVVMTQTPLSLSVGLGDQASISCRSSQSLVHINGSTYLHWYLQKPGQSPKLLIYKIANRFSGVPDRFSGSGSGTDFTLKISRVEAEDLGAYFCSQSTHVPLTFGAGTKLEIK

SEQ ID NO: 41 - protein, 8 aa, Mus musculus
GSIFNKYW

SEQ ID NO: 42 - protein, 8 aa, Mus musculus
ILPGSGST

SEQ ID NO: 43 - protein, 7 aa, Mus musculus
ASRALDY

SEQ ID NO: 44 - DNA, 343 bp, Mus musculus
gaggtgcagctgcaggagtctggagctgagctgatgaagcctggggcctcagtgaagctatcctgcaaggccactggctccatattcaataaatactggatagattgggtcaggcagaggcctggacatggccttgagtggattggagagattttacctggaagtggtagtacaatttacagtgagaagttcaagggcaaggccacaatcactgcagatacatcctccaacacagcctacatgcaactcagcagcctgacatctgaggactctgccgtctattattgtgcaagtcgggcccttgactactggggccaaggcaccactctcactgtctcttcag

SEQ ID NO: 45 - protein, 114 aa, Mus musculus
EVQLQESGAELMKPGASVKLSCKATGSIFNKYWIDWVRQRPGHGLEWIGEILPGSGSTIYSEKFKGKATITADTSSNTAYMQLSSLTSEDSAVYYCASRALDYWGQGTTLTVSS

SEQ ID NO: 46 - protein, 11 aa, Mus musculus
QSLIHSNGNTY

SEQ ID NO: 47 - protein, 3 aa, Mus musculus
QVS

SEQ ID NO: 48 - protein, 9 aa, Mus musculus
SQSTHVPYT

SEQ ID NO: 49 - DNA, 337 bp, Mus musculus
gatgttgtgatgacccaaactccactctccctgcctgtcagtcttggagatcaagcctccatctcttgcagatctagtcagagccttatacacagtaatggaaacacctatttacattggtacctgcagaagccaggccagtctccaaagctcctgatctaccaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaatcaaac

SEQ ID NO: 50 - protein, 112 aa, Mus musculus
DVVMTQTPLSLPVSLGDQASISCRSSQSLIHSNGNTYLHWYLQKPGQSPKLLIYQVSNRFSGVPDRFSGSGSGTDFTLKISRVEAEDLGVYFCSQSTHVPYTFGGGTKLEIK